SEQUENCE LISTING (86 sequences)

SEQ ID NO: 1 - protein, 5 aa, synthetic construct
GSGGS

SEQ ID NO: 2 - protein, 5 aa, synthetic construct
GGGGS

SEQ ID NO: 3 - protein, 4 aa, synthetic construct
GGGS

SEQ ID NO: 4 - protein, 4 aa, synthetic construct
GGSG

SEQ ID NO: 5 - protein, 5 aa, synthetic construct
GGSGG

SEQ ID NO: 6 - protein, 5 aa, synthetic construct
GSGSG

SEQ ID NO: 7 - protein, 5 aa, synthetic construct
GSGGG

SEQ ID NO: 8 - protein, 5 aa, synthetic construct
GGGSG

SEQ ID NO: 9 - protein, 5 aa, synthetic construct
GSSSG

SEQ ID NO: 10 - protein, 6 aa, synthetic construct
FYSSYI

SEQ ID NO: 11 - protein, 10 aa, synthetic construct
SISSSSGSTS

SEQ ID NO: 12 - protein, 12 aa, synthetic construct
FSSYYYTLYWAM

SEQ ID NO: 13 - protein, 5 aa, synthetic construct
SVSSA

SEQ ID NO: 14 - protein, 7 aa, synthetic construct
SASSLYS

SEQ ID NO: 15 - protein, 6 aa, synthetic construct
SIHYPL

SEQ ID NO: 16 - protein, 124 aa, synthetic construct
EISEVQLVESGGGLVQPGGSLRLSCAASGFNFYSSYIHWVRQAPGKGLEWVASISSSSGSTSYADSVKGRFTISADTSKNTAYLQMNSLRAEDTAVYYCARFSSYYYTLYWAMDYWGQGTLVTV

SEQ ID NO: 17 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSIHYPLTFGQGTKVEIKRT

SEQ ID NO: 18 - protein, 234 aa, synthetic construct
EISEVQLVESGGGLVQPGGSLRLSCAASGFNFYSSYIHWVRQAPGKGLEWVASISSSSGSTSYADSVKGRFTISADTSKNTAYLQMNSLRAEDTAVYYCARFSSYYYTLYWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 19 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSIHYPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 251 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSIHYPLTFGQGTKVEIKGTTAASGSSGGSSSGASSSEVQLVESGGGLVQPGGSLRLSCAASGFNFYSSYIHWVRQAPGKGLEWVASISSSSGSTSYADSVKGRFTISADTSKNTAYLQMNSLRAEDTAVYYCARFSSYYYTLYWAMDYWGQGTLVTVSSA

SEQ ID NO: 21 - protein, 6 aa, synthetic construct
FSSSYI

SEQ ID NO: 22 - protein, 10 aa, synthetic construct
SISSSSSSTS

SEQ ID NO: 23 - protein, 12 aa, synthetic construct
YVYSYSQGGPAF

SEQ ID NO: 24 - protein, 5 aa, synthetic construct
SVSSA

SEQ ID NO: 25 - protein, 7 aa, synthetic construct
SASSLYS

SEQ ID NO: 26 - protein, 6 aa, synthetic construct
SWSVPI

SEQ ID NO: 27 - protein, 124 aa, synthetic construct
EISEVQLVESGGGLVQPGGSLRLSCAASGFNFSSSYIHWVRQAPGKGLEWVASISSSSSSTSYADSVKGRFTISADTSKNTAYLQMNSLRAEDTAVYYCARYVYSYSQGGPAFDYWGQGTLVTV

SEQ ID NO: 28 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWSVPITFGQGTKVEIKRT

SEQ ID NO: 29 - protein, 234 aa, synthetic construct
EISEVQLVESGGGLVQPGGSLRLSCAASGFNFSSSYIHWVRQAPGKGLEWVASISSSSSSTSYADSVKGRFTISADTSKNTAYLQMNSLRAEDTAVYYCARYVYSYSQGGPAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 30 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWSVPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 251 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWSVPITFGQGTKVEIKGTTAASGSSGGSSSGASSSEVQLVESGGGLVQPGGSLRLSCAASGFNFSSSYIHWVRQAPGKGLEWVASISSSSSSTSYADSVKGRFTISADTSKNTAYLQMNSLRAEDTAVYYCARYVYSYSQGGPAFDYWGQGTLVTVSSA

SEQ ID NO: 32 - protein, 6 aa, synthetic construct
VYSYSI

SEQ ID NO: 33 - protein, 10 aa, synthetic construct
SIYPSSGYTS

SEQ ID NO: 34 - protein, 12 aa, synthetic construct
GTYYFYFMSWGL

SEQ ID NO: 35 - protein, 5 aa, synthetic construct
SVSSA

SEQ ID NO: 36 - protein, 7 aa, synthetic construct
SASSLYS

SEQ ID NO: 37 - protein, 6 aa, synthetic construct
YFSSLF

SEQ ID NO: 38 - protein, 124 aa, synthetic construct
EISEVQLVESGGGLVQPGGSLRLSCAASGFNVYSYSIHWVRQAPGKGLEWVASIYPSSGYTSYADSVKGRFTISADTSKNTAYLQMNSLRAEDTAVYYCARGTYYFYFMSWGLDYWGQGTLVTV

SEQ ID NO: 39 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYFSSLFTFGQGTKVEIKRT

SEQ ID NO: 40 - protein, 234 aa, synthetic construct
EISEVQLVESGGGLVQPGGSLRLSCAASGFNVYSYSIHWVRQAPGKGLEWVASIYPSSGYTSYADSVKGRFTISADTSKNTAYLQMNSLRAEDTAVYYCARGTYYFYFMSWGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 41 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYFSSLFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 251 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYFSSLFTFGQGTKVEIKGTTAASGSSGGSSSGASSSEVQLVESGGGLVQPGGSLRLSCAASGFNVYSYSIHWVRQAPGKGLEWVASIYPSSGYTSYADSVKGRFTISADTSKNTAYLQMNSLRAEDTAVYYCARGTYYFYFMSWGLDYWGQGTLVTVSSA

SEQ ID NO: 43 - protein, 6 aa, synthetic construct
VYSSSI

SEQ ID NO: 44 - protein, 10 aa, synthetic construct
SIYSYSSSTS

SEQ ID NO: 45 - protein, 13 aa, synthetic construct
FDWYQRYSFYEAM

SEQ ID NO: 46 - protein, 5 aa, synthetic construct
SVSSA

SEQ ID NO: 47 - protein, 7 aa, synthetic construct
SASSLYS

SEQ ID NO: 48 - protein, 6 aa, synthetic construct
SYFYPV

SEQ ID NO: 49 - protein, 125 aa, synthetic construct
EISEVQLVESGGGLVQPGGSLRLSCAASGFNVYSSSIHWVRQAPGKGLEWVASIYSYSSSTSYADSVKGRFTISADTSKNTAYLQMNSLRAEDTAVYYCARFDWYQRYSFYEAMDYWGQGTLVTV

SEQ ID NO: 50 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYFYPVTFGQGTKVEIKRT

SEQ ID NO: 51 - protein, 235 aa, synthetic construct
EISEVQLVESGGGLVQPGGSLRLSCAASGFNVYSSSIHWVRQAPGKGLEWVASIYSYSSSTSYADSVKGRFTISADTSKNTAYLQMNSLRAEDTAVYYCARFDWYQRYSFYEAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 52 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYFYPVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 252 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYFYPVTFGQGTKVEIKGTTAASGSSGGSSSGASSSEVQLVESGGGLVQPGGSLRLSCAASGFNVYSSSIHWVRQAPGKGLEWVASIYSYSSSTSYADSVKGRFTISADTSKNTAYLQMNSLRAEDTAVYYCARFDWYQRYSFYEAMDYWGQGTLVTVSSA

SEQ ID NO: 54 - protein, 6 aa, synthetic construct
VSSSYI

SEQ ID NO: 55 - protein, 10 aa, synthetic construct
YIYPYSGYTS

SEQ ID NO: 56 - protein, 13 aa, synthetic construct
NYGGYPRKYAGAF

SEQ ID NO: 57 - protein, 5 aa, synthetic construct
SVSSA

SEQ ID NO: 58 - protein, 7 aa, synthetic construct
SASSLYS

SEQ ID NO: 59 - protein, 6 aa, synthetic construct
SYGSLI

SEQ ID NO: 60 - protein, 125 aa, synthetic construct
EISEVQLVESGGGLVQPGGSLRLSCAASGFNVSSSYIHWVRQAPGKGLEWVAYIYPYSGYTSYADSVKGRFTISADTSKNTAYLQMNSLRAEDTAVYYCARNYGGYPRKYAGAFDYWGQGTLVTV

SEQ ID NO: 61 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYGSLITFGQGTKVEIKRT

SEQ ID NO: 62 - protein, 235 aa, synthetic construct
EISEVQLVESGGGLVQPGGSLRLSCAASGFNVSSSYIHWVRQAPGKGLEWVAYIYPYSGYTSYADSVKGRFTISADTSKNTAYLQMNSLRAEDTAVYYCARNYGGYPRKYAGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 63 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYGSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 252 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYGSLITFGQGTKVEIKGTTAASGSSGGSSSGASSSEVQLVESGGGLVQPGGSLRLSCAASGFNVSSSYIHWVRQAPGKGLEWVAYIYPYSGYTSYADSVKGRFTISADTSKNTAYLQMNSLRAEDTAVYYCARNYGGYPRKYAGAFDYWGQGTLVTVSSA

SEQ ID NO: 65 - protein, 6 aa, synthetic construct
FSSSYI

SEQ ID NO: 66 - protein, 10 aa, synthetic construct
SISSSSGSTS

SEQ ID NO: 67 - protein, 12 aa, synthetic construct
FSYMSGSVFWAL

SEQ ID NO: 68 - protein, 5 aa, synthetic construct
SVSSA

SEQ ID NO: 69 - protein, 7 aa, synthetic construct
SASSLYS

SEQ ID NO: 70 - protein, 6 aa, synthetic construct
SSWFPI

SEQ ID NO: 71 - protein, 124 aa, synthetic construct
EISEVQLVESGGGLVQPGGSLRLSCAASGFNFSSSYIHWVRQAPGKGLEWVASISSSSGSTSYADSVKGRFTISADTSKNTAYLQMNSLRAEDTAVYYCARFSYMSGSVFWALDYWGQGTLVTV

SEQ ID NO: 72 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSWFPITFGQGTKVEIKRT

SEQ ID NO: 73 - protein, 234 aa, synthetic construct
EISEVQLVESGGGLVQPGGSLRLSCAASGFNFSSSYIHWVRQAPGKGLEWVASISSSSGSTSYADSVKGRFTISADTSKNTAYLQMNSLRAEDTAVYYCARFSYMSGSVFWALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 74 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSWFPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 251 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSWFPITFGQGTKVEIKGTTAASGSSGGSSSGASSSEVQLVESGGGLVQPGGSLRLSCAASGFNFSSSYIHWVRQAPGKGLEWVASISSSSGSTSYADSVKGRFTISADTSKNTAYLQMNSLRAEDTAVYYCARFSYMSGSVFWALDYWGQGTLVTVSSA

SEQ ID NO: 76 - protein, 6 aa, synthetic construct
LSSSSI

SEQ ID NO: 77 - protein, 10 aa, synthetic construct
SISSYSGYTY

SEQ ID NO: 78 - protein, 12 aa, synthetic construct
YSYKYWGWGEGM

SEQ ID NO: 79 - protein, 5 aa, synthetic construct
SVSSA

SEQ ID NO: 80 - protein, 7 aa, synthetic construct
SASSLYS

SEQ ID NO: 81 - protein, 6 aa, synthetic construct
GYYDLI

SEQ ID NO: 82 - protein, 124 aa, synthetic construct
EISEVQLVESGGGLVQPGGSLRLSCAASGFNLSSSSIHWVRQAPGKGLEWVASISSYSGYTYYADSVKGRFTISADTSKNTAYLQMNSLRAEDTAVYYCARYSYKYWGWGEGMDYWGQGTLVTV

SEQ ID NO: 83 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQGYYDLITFGQGTKVEIKRT

SEQ ID NO: 84 - protein, 234 aa, synthetic construct
EISEVQLVESGGGLVQPGGSLRLSCAASGFNLSSSSIHWVRQAPGKGLEWVASISSYSGYTYYADSVKGRFTISADTSKNTAYLQMNSLRAEDTAVYYCARYSYKYWGWGEGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 85 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQGYYDLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 251 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQGYYDLITFGQGTKVEIKGTTAASGSSGGSSSGASSSEVQLVESGGGLVQPGGSLRLSCAASGFNLSSSSIHWVRQAPGKGLEWVASISSYSGYTYYADSVKGRFTISADTSKNTAYLQMNSLRAEDTAVYYCARYSYKYWGWGEGMDYWGQGTLVTVSSA

(19) United States
(12) Patent Application Publication
(10) Pub. No.: US 2026/0226157 A1
(43) Pub. Date: Aug. 6, 2026

(54) ANTIGEN-BINDING PROTEINS AND USE THEREOF

(71) Applicant: THE UNIVERSITY OF CHICAGO, Chicago, IL (US)

(72) Inventors: Anthony KOSSIAKOFF, Chicago, IL (US); Kelly O’LEARY, Chicago, IL (US); Tomasz SLEZAK, Chicago, IL (US)

(73) Assignee: THE UNIVERSITY OF CHICAGO, Chicago, IL (US)

(21) Appl. No.: 19/464,995

(22) Filed: Jan. 30, 2026

Related U.S. Application Data
(60) Provisional application No. 63/752,110, filed on Jan. 31, 2025.

(51) Int. Cl. C07K 16/28; A61K 45/06; G01N 33/543; G01N 33/575

(52) U.S. Cl. CPC C07K 16/28; A61K 45/06; G01N 33/543; G01N 33/5759; C07K 2317/14; C07K 2317/24; C07K 2317/31; C07K 2317/565; C07K 2317/76; C07K 2317/92; C07K 2319/09

(57) ABSTRACT

Aspects of the present disclosure are directed to mTOR-targeting polypeptides, including antibodies, antibody-drug conjugates, antibody fragments, antibody-like molecules, and chimeric receptors. Also disclosed herein are nucleic acids encoding for such mTOR-targeting polypeptides and cells comprising such nucleic acids. Also described are methods for treatment of diseases using mTOR-targeting polypeptides.

### CROSS REFERENCE TO RELATED APPLICATIONS

This application claims priority of U.S. Provisional Application No. 63/752,110 filed Jan. 31, 2025, which is hereby incorporated by reference in its entirety.

### BACKGROUND

### I. Field of the Invention

Aspects of the invention relate to at least the fields of molecular biology and medicine.

### II. Sequence Listing

The instant application contains a Sequence Listing which has been submitted electronically in XML format and is hereby incorporated by reference in its entirety for all purposes. The XML copy, created on Jan. 28, 2026, is referred to as “ARCDP0868US Sequence Listing” and is 81,404 bytes in size.

### III. Background

Subcellular compartmentalization is integral to the spatial regulation of mechanistic target of rapamycin (mTOR) signaling. However, the biological outputs associated with location-specific mTOR signaling events are poorly understood and challenging to decouple.

Currently, there are no existing technologies that describe fully genetically encoded modulators of mTOR signaling. Current approaches to perturb the mTOR signaling network include small molecule inhibitors, genetic ablation, or chemical-genetic inhibition. These approaches lack the potential for fine-tuned spatial control over mTOR signaling in select subcellular compartments.

Mechanistic target of rapamycin (mTOR) is a serine/threonine protein kinase that regulates cellular growth and metabolism through multifaceted signaling phenotypes in specific tissues, cell types, and subcellular compartments (1-4). mTOR serves as the catalytic subunit in two functionally distinct assemblies, mTOR complex 1 (mTORC1) and mTOR complex 2 (mTORC2) (5-7), which phosphorylate independent sets of substrates in response to nutrient and growth factor stimulation (8). Numerous studies have highlighted the robust therapeutic potential of systemic mTOR inhibition by the natural product rapamycin or ATP-competitive inhibitors in diverse model organisms (9-16). However, undesirable side effects or feedback loops resulting from on-target mTORC1/mTORC2 inhibition have limited the clinical efficacy of mTOR inhibitors for various diseases such as diabetes, cancer, neurodegenerative disorders, and metabolic syndromes (17-19). Nevertheless, the emerging concept that tissue-specific (20, 21) and subcellular (22, 23) mTOR signaling networks are functionally linked to distinct biological outputs represents a promising path forward to decouple favorable therapeutic effects from toxicities associated with systemic mTOR inhibition.

How the subcellular spatial regulation of mTOR signaling influences mechanistically diverse processes such as nutrient-specific signal transduction, macromolecular metabolism, or transcriptional regulation remains elusive (24). While canonical mTORC1 signaling occurs at the surface of lysosomes (25, 26), mTORC1 activity has also been reported at noncanonical locations such as the plasma membrane, mitochondria, and nucleus (27-29). In fact, recent studies have pointed to mTOR as a driver of prostate cancer aggressiveness by rewiring androgen-dependent transcriptional networks from inside the nucleus (30-34). Uncovering whether mTOR activity is functionally relevant inside the nucleus, and if so, whether nuclear mTOR is active toward a defined set of substrates could provide valuable mechanistic insight to improve current therapeutic strategies. However, shortcomings in techniques to execute functional mTOR perturbations with subcellular spatial resolution represent a key technological barrier to our understanding.

### SUMMARY

Certain aspects herein relate to the development of a general framework to modulate mTORFRB-mediated substrate recruitment in a rapamycin-like fashion using genetically encodable scFv-based intrabodies. Structural and functional aspects of two conformation-specific intrabodies provide molecular insight into the allosteric regulation of the mTORC1 architecture mediated through subtle conformational rearrangements in the mTORFRB domain. Furthermore, aspects herein decouple cytosolic mTOR signaling from nuclear mTOR signaling through location-specific intrabody-based functional perturbations, demonstrating that subcellular mTOR networks can indeed be manipulated independently. Accordingly, aspects herein establish a novel approach to decouple spatially restricted functions of the mTOR signaling network systematically.

To address certain needs in the art, the inventors have generated a diverse number of antigen-binding proteins, including Fabs and antibodies, that bind to the FKBP12-Rapamycin binding (FRB) domain of mTOR. The disclosure describes novel antibody and antigen binding fragments, as well as methods of using these antibodies and fragments. Also described are polypeptides comprising the antigen binding fragment(s) of the disclosure, and compositions comprising the polypeptides, antibodies, and/or antigen binding fragments of the disclosure. Also described are nucleic acids encoding an antibody or antigen binding fragment of the disclosure. The disclosure also relates to an antibody comprising at least one polypeptide with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of the sequences of Table 1. Also described are nucleic acids, vectors, or expression vectors encoding antibodies of the disclosure and host cells comprising polypeptides, nucleic acids, vectors, antibodies, or antigen binding fragments of the disclosure. The nucleic acids of the disclosure may be DNA or RNA.

Also described is a method of a making a cell comprising transferring one or more nucleic acid(s) encoding an antibody of the disclosure into a cell. The method may comprise or further comprise culturing the cell under conditions that allow for expression of a polypeptide from the nucleic acid. The method may further comprise isolating the expressed polypeptide. The cell may be further defined as a human cell, B cell, T cell, Chinese hamster ovary, NS0 murine myeloma cell, PER.C6 cell, or a cell described herein.

The disclosure describes a method for treating or preventing a disease indicated for an immunotherapy in a subject, the method comprising administering to the subject an antibody, antigen binding fragment, polypeptide, nucleic acid, or host cell of the disclosure. Also described is a method for evaluating a sample from a subject, the method comprising contacting a biological sample from the subject, or extract thereof, with at least one antibody, antigen binding fragment, polypeptide, composition, or host cell of the disclosure. The antibodies, antigen binding fragments, or compositions of the disclosure may be used to treat a subject having a cancer, an infection, or any other disease that is indicated for an immunotherapy.

Methods include a method for treating or preventing cancer in a subject, a method for treating or preventing a metabolic disease, a method for treating or preventing a disease associated with mTOR, a method for treating or preventing a disease associated with rapamycin. In some aspects, the method comprises administering to the subject an antibody, antigen binding fragment, polypeptide, nucleic acid, or host cell of the disclosure. Also described is a method for evaluating a sample from a subject, the method comprising contacting a biological sample from the subject, or extract thereof, with at least one antibody, antigen binding fragment, or polypeptide, composition, or host cell of the disclosure. Also disclosed is a method for diagnosing cancer in a subject, the method comprising contacting a biological sample from the subject, or extract thereof, with at least one antibody, antigen binding fragment, composition, or polypeptide of any one of the disclosure. The antibodies, antigen binding fragments, or compositions of the disclosure may be used to treat a subject having cancer.

Provided by the disclosure is an antibody or antigen binding fragment comprising a heavy chain variable region and a light chain variable region, wherein the heavy chain variable region comprises a HCDR1, HCDR2, and HCDR3 from a heavy chain variable region of an antibody clone of Table 1 and wherein the light chain variable region comprises a LCDR1, LCDR2, and LCDR3 from the light chain variable region of the same clone of Table 1. The disclosure also describes an antibody or antigen binding fragment comprising a heavy chain variable region and a light chain variable region, wherein the heavy chain variable region comprises a HCDR1, HCDR2, and HCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a HCDR1, HCDR2, and HCDR3 from a heavy chain variable region of an antibody clone of Table 1 and wherein the light chain variable region comprises a LCDR1, LCDR2, and LCDR3 having or having at least least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of the same clone of Table 1. The HCDR1, HCDR2, HCDR3, LCDR1, LCDR2, and/or LCDR3 may be determined from the variable region sequences by methods known in the art. The CDR may be a HCDR1, HCDR2, HCDR3, LCDR1, LCDR2, and/or LCDR3 determined by the Chothia method. The CDR may be a HCDR1, HCDR2, HCDR3, LCDR1, LCDR2, and/or LCDR3 determined by the Kabat method. The CDR may be a HCDR1, HCDR2, HCDR3, LCDR1, LCDR2, and/or LCDR3 determined by the IMGT method.

The disclosure provides for an antibody or antigen binding fragment in which the HCDR1, HCDR2, HCDR3, LCDR1, LCDR2, and LCDR3 each comprise an amino acid sequence that has at least 80% sequence identity to an HCDR1, HCDR2, HCDR3, LCDR1, LCDR2, and LCDR3 of Table 1, wherein the HCDR1, HCDR2, HCDR3, LCDR1, LCDR2, and LCDR3 are from the same antibody clone. The HCDR1, HCDR2, HCDR3, LCDR1, LCDR2, and LCDR3 may each comprise an amino acid sequence that has or has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HCDR1, HCDR2, HCDR3, LCDR1, LCDR2, and LCDR3 of Table 1, wherein the HCDR1, HCDR2, HCDR3, LCDR1, LCDR2, and LCDR3 are from the same antibody clone. The HCDR1, HCDR2, HCDR3, LCDR1, LCDR2, and LCDR3 may each comprise the amino acid sequence of an HCDR1, HCDR2, HCDR3, LCDR1, LCDR2, and LCDR3 of Table 1, wherein the HCDR1, HCDR2, HCDR3, LCDR1, LCDR2, and LCDR3 are from the same antibody clone.

The disclosure describes an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the HCDR1, HCR2, HCR3 from a heavy chain variable region of an antibody clone of Table 1 and wherein the light chain variable region comprises a LCDR1, LCDR2, and LCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LCDR1, LCDR2, and LCDR3 from the light chain variable region of the same antibody clone of Table 1. The antibody or antigen binding fragment may comprise a heavy chain variable region and alight chain variable region, wherein the heavy chain variable region comprises a HCDR1, HCDR2, and HCDR3 having or having at least 80% sequence identity to the HCDR1, HCR2, HCR3 from a heavy chain variable region of an antibody clone of Table 1 and wherein the light chain variable region comprises a LCDR1, LCDR2, and LCDR3 having at least 80% sequence identity to the LCDR1, LCDR2, and LCDR3 from the light chain variable region of the same antibody clone of Table 1. In some aspects, the heavy chain variable region comprises a HCDR1, HCDR2, and HCDR3 having the amino acid sequence of an of a HCDR1, HCDR2, and HCDR3 of a clone of Table 1 and the light chain variable region comprises a LCDR1, LCDR2, and LCDR3 comprising the amino acid sequence of the LCDR1, LCDR2, and LCDR3 from the light chain variable region of the same clone of Table 1.

In certain aspects, the antibody or antigen binding fragment is an intracellular antibody or an intrabody. In certain aspects, the intracellular antibody or intrabody is a modified antibody or antigen binding fragment that can be expressed inside of a cell, including any cell described herein. The antibody or antigen binding fragment may be modified as described herein.

The polypeptides of the disclosure may comprise at least two antigen binding fragments, wherein each antigen binding fragment is independently selected from an antigen binding fragment of the disclosure. The polypeptide may be multivalent. The polypeptide may be multispecific. The polypeptide may be bispecific. The polypeptide may comprise, comprise at least, or comprise at most 1, 2, 3, 4, 5, 6, 7, 8, 9, or 10 antigen binding regions. Each antigen binding region may be independently selected from an antigen binding region of the disclosure. The polypeptide may have repeated units of the same antigen binding region, such as at least, at most, or exactly 1, 2, 3, 4, 5, 6, 7, 8, 9, or 10 repeated units.

The heavy chain variable region may comprise an amino acid sequence with at least 80% sequence identity to a heavy chain variable region of an antibody clone of Table 1 and/or the light chain variable region may comprise an amino acid sequence with at least 80% sequence identity to the light chain variable region of the same antibody clone of Table 1. The heavy chain variable region may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain variable region of an antibody clone of Table 1 and/or the light chain variable region may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain variable region of the same antibody clone of Table 1. The heavy chain variable region may comprise the amino acid sequence of a heavy chain variable region of an antibody clone of Table 1 and/or the light chain variable region may comprise the amino acid sequence of the same antibody clone of Table 1. The antibody or antigen binding fragment may comprise a heavy chain and a light chain and wherein the heavy chain comprises an amino acid sequence with at least 70% sequence identity to a heavy chain of an antibody clone of Table 1 and the light chain comprises an amino acid sequence with at least 70% sequence identity to the light chain of the same antibody clone of Table 1. The antibody or antigen binding fragment may comprise a heavy chain and a light chain and wherein the heavy chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain of an antibody clone of Table 1 and the light chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain of the same antibody clone of Table 1. The antibody or antigen binding fragment may comprise a heavy chain and a light chain and wherein the heavy chain comprises the amino acid sequence of an antibody clone of Table 1 and the light chain comprises the amino acid sequence of the same antibody clone of Table 1.

The heavy chain variable region may comprise a heavy chain framework region that has or has at least 80% sequence identity to a heavy chain framework region of an antibody clone of Table 1 and the light chain variable region comprises a light chain framework region that has or has at least 80% sequence identity to a light chain framework region of the same antibody clone of Table 1. The heavy chain variable region may comprise a heavy chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain framework region of an antibody clone of Table 1 and the light chain variable region comprises a light chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a light chain framework region of the same antibody clone of Table 1.

The heavy chain variable region may comprise at least 70% sequence identity to the heavy chain variable region of an antibody clone of Table 1 and the light chain variable region comprises at least 70% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. The heavy chain variable region may comprise at least 75% sequence identity to the heavy chain variable region of an antibody clone of Table 1 and the light chain variable region comprises at least 75% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. The heavy chain variable region may comprise at least 80% sequence identity to the heavy chain variable region of an antibody clone of Table 1 and the light chain variable region comprises at least 80% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. The heavy chain variable region may comprise at least 85% sequence identity to the heavy chain variable region of an antibody clone of Table 1 and the light chain variable region comprises at least 85% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. The heavy chain variable region may comprise at least 90% sequence identity to the heavy chain variable region of an antibody clone of Table 1 and the light chain variable region comprises at least 90% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. The heavy chain variable region may comprise at least 95% sequence identity to the heavy chain variable region of an antibody clone of Table 1 and the light chain variable region comprises at least 95% sequence identity to the light chain variable region of the same antibody clone of Table 1, and wherein the heavy chain and light chain comprise 100% sequence identity to each of the three heavy chain CDRs and three light chain CDRs from the same antibody clone of Table 1.

The antigen binding fragment may be at least 2, 3, 4, 5, or 6 scFv, F(ab′)2, Fab′, Fab, Fv, or rIgG, or combinations thereof. The polypeptide and/or antigen binding fragments of the disclosure may comprise a linker between a heavy chain and light chain variable region or between antigen binding fragments. The linker may be a flexible linker. Exemplary flexible linkers include glycine polymers (G)n, glycine-serine polymers (including, for example, (GS)n, (GSGGS(SEQ ID NO: 1))n, (G4S(SEQ ID NO: 2))n and (GGGS(SEQ ID NO: 3))n, where n is an integer of at least one. n may be at least, at most, or exactly 1, 2, 3, 4, 5, 6, 7, 8, 9, or 10 (or any derivable range therein). Glycine-alanine polymers, alanine-serine polymers, and other flexible linkers known in the art and may be used as a linker in the polypeptides of the disclosure. Exemplary linkers can comprise or consist of GGSG (SEQ ID NO: 4), GGSGG (SEQ ID NO: 5), GSGSG (SEQ ID NO: 6), GSGGG (SEQ ID NO: 7), GGGSG (SEQ ID NO: 8), GSSSG (SEQ ID NO: 9), and the like.

In certain aspects, the antibody or antigen binding fragment comprises a subcellular localization domain. In certain aspects, the subcellular localization domain comprises a nuclear localization signal domain, a nuclear export signal domain, a lyn kinase motif, or a Yxxφ motif. The subcellular localization signal domain can, in certain aspects, allow for the

The antibody or antigen binding fragment of the disclosure may be human, chimeric, or humanized. The antibody, or antigen binding fragment may bind mTOR, including a FKBP12-Rapamycin binding domain of mTOR, with a K_{D} of about 10⁻⁶ M/L or M to about 10⁻¹² MIL or M. The antibody or antigen binding fragment may bind mTOR, including a FKBP12-Rapamycin binding domain of mTOR, with a K_{D} of about, a K_{D} of at least, or a K_{D} of at most 10, 10⁻¹, 10⁻², 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, or 10⁻¹⁸ (or any derivable range therein) M, M/L, μM, nM, or pM. The antibody or antigen binding fragment may bind mTOR, including a FKBP12-Rapamycin binding domain of mTOR, with a K_{D} of less than 20, 19, 18, 17, 16, 15, 14, 13, 12, 11, 10, 9, 8, 7, 6, 5, 4, 3, 2, or 1 nM, or any range or value derivable therein. The antibody or antigen binding fragment may bind mTOR, including a FKBP12-Rapamycin binding domain of mTOR, with a K_{D} of less than 2 nM. The antibody or antigen binding fragment may bind mTOR, including a FKBP12-Rapamycin binding domain of mTOR, with a K_{D} of less than 1 nM. The antibody or antigen binding fragment may bind mTOR, including a FKBP12-Rapamycin binding domain of mTOR, with a K_{D} of less than 0.5 nM. The antibody or antigen binding fragment may be further defined as a human antibody or antigen binding fragment, humanized antibody or antigen binding fragment, recombinant antibody or antigen binding fragment, chimeric antibody or antigen binding fragment, an antibody or antigen binding fragment derivative, a veneered antibody or antigen binding fragment, a diabody, a monoclonal antibody or antigen binding fragment, a single domain antibody, or a single chain antibody. The antigen binding fragment may be further defined as a single chain variable fragment (scFv), F(ab′)₂, Fab′, Fab, Fv, or rIgG. The antibody, antigen binding fragment, or polypeptide may be operatively linked to a detectable label. Detectable labels are described herein.

Also described herein are multi-specific antibodies and polypeptides. Accordingly, bivalent or bispecific antibodies that comprise two antigen binding fragments, wherein the antigen binding fragment is two of the same antigen binding fragments or two different antigen binding fragments are provided by the description. The disclosure also provides for multi-specific polypeptides. Also described are polypeptides comprising at least 2, 3, 4, 5, or 6 antigen binding fragments. The antigen binding fragment may be at least 2, 3, r, 5, or 6 scFv, F(ab′)₂, Fab′, Fab, Fv, or rIgG, or combinations thereof.

Compositions may comprise more than one antibody and/or antigen binding fragment of the disclosure. Accordingly, compositions of the disclosure may comprise, may comprise at least, or may comprise at most 1, 2, 3, 4, 5, 6, 7, 8, 9, or 10 antibodies and/or antigen binding fragments of the disclosure.

In embodiments of the disclosure, it is also contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 polypeptides of Table 1 may be excluded.

The subject may be further defined as a human subject or a mammalian subject. The subject may be a laboratory or veterinary animal, such as a pig, horse, cat, cow, rabbit, mouse, rat, or dog. The subject may have one or more symptom of cancer. The subject may not have any symptoms of cancer. The subject may be one that has been diagnosed with cancer. The subject may be one that has not been diagnosed with cancer. The subject may be one that has been previously treated for cancer. The subject may be one that has one or more symptoms of an infection. The subject may be one that does not have one or more symptoms of an infection. The subject may be one that has been diagnosed with an infection. The subject may be one that has not been diagnosed with an infection. The subject may be one that has symptoms of a disease treated with an immunotherapy. The subject may be one that does not have symptoms of a disease treated with an immunotherapy. The subject may be one that has been diagnosed with a disease indicated for an immunotherapy. The subject may be one that has not been diagnosed with a disease indicated for an immunotherapy.

The subject may be administered an additional therapy. The subject may be one that is resistant or has been determined to be resistant to the previous therapy. The additional therapy may comprise one or more of radiotherapy, chemotherapy, and immunotherapy.

The method may further comprise incubating the antibody, antigen binding fragment, or polypeptide under conditions that allow for the binding of the antibody, antigen binding fragment, or polypeptide to antigens in the biological sample or extract thereof. The method may further comprise detecting the binding of an antigen to the antibody, antigen binding fragment, or polypeptide. The method may further comprise contacting the biological sample with at least one capture antibody, antigen, or polypeptide. The at least one capture antibody, antigen binding fragment, or polypeptide may be an antibody, polypeptide, or antigen binding fragment of the disclosure. The capture antibody may be linked or operatively linked to a solid support. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. The biological sample may comprise a blood sample, urine sample, fecal sample, or nasopharyngeal sample. The at least one antibody, antigen binding fragment, or polypeptide may be operatively linked to a detectable label. The method may further comprise incubating the antibody, antigen binding fragment, or polypeptide under conditions that allow for the binding of the antibody, antigen binding fragment, or polypeptide to antigens in the biological sample or extract thereof. The method may further comprise detecting the binding of an antigen to the antibody, antigen binding fragment, or polypeptide. The method may further comprise contacting the biological sample with at least one capture antibody, antigen, or polypeptide. The biological sample may comprise a blood sample, urine sample, fecal sample, or nasopharyngeal sample.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of clone T1, respectively and the LCDR1, LCDR2, and LCDR3 comprises an amino acid sequence of clone T1, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of clone T2, respectively and the LCDR1, LCDR2, and LCDR3 comprises an amino acid sequence of clone T2, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of clone T3, respectively and the LCDR1, LCDR2, and LCDR3 comprises an amino acid sequence of clone T3, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of clone T4, respectively and the LCDR1, LCDR2, and LCDR3 comprises an amino acid sequence of clone T4, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of clone T5, respectively and the LCDR1, LCDR2, and LCDR3 comprises an amino acid sequence of clone T5, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of clone T6, respectively and the LCDR1, LCDR2, and LCDR3 comprises an amino acid sequence of clone T6, respectively.

The disclosure describes an antibody, antigen binding fragment, or polypeptide comprising a heavy chain variable region and a light chain variable region of any one of clones T1, T2, T3, T4, T5, or T6.

Also disclosed are any of the following enumerated Aspects:

Aspect 1 includes an antibody or antigen binding fragment comprising a heavy chain variable region and a light chain variable region, wherein the heavy chain variable region comprises an HCDR1, HCDR2, and HCDR3 having at least 80% sequence identity to the HCDR1, HCDR2, and HCDR3 from a heavy chain variable region of an antibody clone of Table 1 and wherein the light chain variable region comprises an LCDR1, LCDR2, and LCDR3 having at least 80% sequence identity to the LCDR1, LCDR2, and LCDR3 from the light chain variable region of the same antibody clone of Table 1.

Aspect 2 depends upon Aspect 1, wherein the heavy chain variable region comprises an HCDR1, HCDR2, and HCDR3 having the amino acid sequence of an of a HCDR1, HCDR2, and HCDR3 of a clone of Table 1 and wherein the light chain variable region comprises an LCDR1, LCDR2, and LCDR3 comprising the amino acid sequence of the LCDR1, LCDR2, and LCDR3 from the light chain variable region of the same clone of Table 1.

Aspect 3 depends upon Aspect 1 or Aspect 2, wherein the HCDR1, HCDR2, HCDR3, LCDR1, LCDR2, and LCDR3 each comprise an amino acid sequence that has at least 80% sequence identity to an HCDR1, HCDR2, HCDR3, LCDR1, LCDR2, and LCDR3 of Table 1, wherein the HCDR1, HCDR2, HCDR3, LCDR1, LCDR2, and LCDR3 are from the same antibody clone.

Aspect 4 depends upon Aspect 1 or Aspect 2, wherein the HCDR1, HCDR2, HCDR3, LCDR1, LCDR2, and LCDR3 each comprise the amino acid sequence of an HCDR1, HCDR2, HCDR3, LCDR1, LCDR2, and LCDR3 of Table 1, wherein the HCDR1, HCDR2, HCDR3, LCDR1, LCDR2, and LCDR3 are from the same antibody clone.

Aspect 5 depends upon any one of Aspects 1 to 4, wherein the heavy chain variable region comprises an amino acid sequence with at least 80% sequence identity to a heavy chain variable region of an antibody clone of Table 1 and/or the light chain variable region comprises an amino acid sequence with at least 80% sequence identity to the light chain variable region of the same antibody clone of Table 1.

Aspect 6 depends upon Aspect 5, wherein the heavy chain variable region comprises the amino acid sequence of a heavy chain variable region of an antibody clone of Table 1 and/or the light chain variable region comprises the amino acid sequence of the same antibody clone of Table 1.

Aspect 7 depends upon any one of Aspects 1 to 6, wherein the antibody or antigen binding fragment comprises a full heavy chain and a full light chain with at least 80% sequence identity to an antibody clone of Table 1.

Aspect 8 depends upon any one of Aspects 1 to 6, wherein the antibody or antigen binding fragment comprises a full heavy chain and a full light chain of an antibody clone of Table 1.

Aspect 9 depends upon any one of Aspects 1 to 6, wherein the antibody comprises a heavy chain and a light chain and wherein the heavy chain comprises an amino acid sequence with at least 70% sequence identity to a heavy chain of an antibody clone of Table 1 and the light chain comprises an amino acid sequence with at least 70% sequence identity to the light chain of the same antibody clone of Table 1.

Aspect 10 depends upon Aspect 9, wherein the antibody comprises a heavy chain and a light chain and wherein the heavy chain comprises the amino acid sequence of an antibody clone of Table 1 and the light chain comprises the amino acid sequence of the same antibody clone of Table 1.

Aspect 11 depends upon any one of Aspects 1 to 10, wherein the antibody is human, chimeric, or humanized.

Aspect 12 depends upon any one of Aspects 1 to 11, wherein the antibody or antigen binding fragment binds an FKBP12-rapamycin binding domain of an mTOR protein with a KD of about 10⁶ MIL to about 10¹² M/L.

Aspect 13 depends upon any one of Aspects 1 to 12, wherein the antibody is a neutralizing antibody.

Aspect 14 depends upon any one of Aspects 1 to 13, wherein the antibody is a human antibody, humanized antibody, recombinant antibody, chimeric antibody, an antibody derivative, a veneered antibody, a diabody, a monoclonal antibody, a single domain antibody, or a single chain antibody.

Aspect 15 depends upon any one of Aspects 1 to 13, wherein the antigen binding fragment is a single chain variable fragment (scFv), F(ab′)2, Fab′, Fab, Fv, or rIgG.

Aspect 16 depends upon any one of Aspects 1 to 15, further comprising a subcellular localization domain.

Aspect 17 depends upon Aspect 16, wherein the subcellular localization domain comprises a nuclear localization signal domain, a nuclear export signal domain, a lyn kinase motif, or a Yxxφ motif.

Aspect 18 includes a polypeptide comprising the antigen binding fragment of any one of Aspects 1 to 17.

Aspect 19 depends upon Aspect 18, wherein the polypeptide comprises at least two antigen binding fragments, wherein each antigen binding fragment is independently selected from any one of Aspects 1 to 17.

Aspect 20 depends upon Aspect 18 or Aspect 19, wherein the polypeptide is multivalent.

Aspect 21 depends upon any one of Aspects 18 to 20, wherein the polypeptide is bispecific.

Aspect 22 includes a composition comprising the antibody or antigen binding fragment of any one of Aspects 1 to 17 or the polypeptide of any one of Aspects 18 to 21.

Aspect 23 depends upon Aspect 22, wherein the composition comprises a pharmaceutical excipient.

Aspect 24 depends upon Aspect 22 or Aspect 23, wherein the composition further comprises an adjuvant.

Aspect 25 depends upon any one of Aspects 22 to 24, wherein the composition is formulated for parenteral, intravenous, subcutaneous, intramuscular, or intranasal administration.

Aspect 26 depends upon any one of Aspects 22 to 25, wherein the composition comprises at least two antibodies or antigen binding fragments.

Aspect 27 includes one or more nucleic acids encoding the antibody or antigen binding fragment of any one of Aspects 1 to 17 or the polypeptide of any one of Aspects 18 to 21.

Aspect 28 includes a vector comprising the nucleic acid(s) of Aspect 27.

Aspect 29 includes a host cell comprising the nucleic acid of Aspect 27 or the vector of Aspect 28.

Aspect 30 depends upon Aspect 29, wherein the host cell is a human cell, B cell, T cell, Chinese hamster ovary, NS0 murine myeloma cell, or PER.C6 cell.

Aspect 31 includes a method of making a cell comprising transferring the nucleic acid(s) of Aspect 27 or the vector of Aspect 28 into a cell.

Aspect 32 depends upon Aspect 31, wherein the method further comprises culturing the cell under conditions that allow for expression of a polypeptide from the nucleic acid.

Aspect 33 depends upon Aspect 32, wherein the method further comprises isolating the expressed polypeptide.

Aspect 34 depends upon any one of Aspects 31 to 33, wherein the cell is a human cell, B cell, T cell, Chinese hamster ovary, NS0 murine myeloma cell, or PER.C6 cell.

Aspect 35 includes a method for producing a polypeptide comprising transferring the nucleic acid(s) of Aspect 27 or the vector of Aspect 28 into a cell and isolating polypeptides expressed from the nucleic acid.

Aspect 36 depends upon Aspect 35, wherein the cell is a human cell, B cell, T cell, Chinese hamster ovary, NS0 murine myeloma cell, or PER.C6 cell.

Aspect 37 includes a method for treating or preventing a disease in a subject, the method comprising administering to the subject the antibody or antigen binding fragment of any one of Aspects 1 to 17, the polypeptide of any one of Aspects 18 to 21, the composition of any one of Aspects 22 to 26, or the host cell of Aspect 29 or Aspect 30.

Aspect 38 depends upon Aspect 37, wherein the subject is a human subject.

Aspect 39 depends upon Aspect 37 or Aspect 38, wherein the disease is a cancer.

Aspect 40 depends upon Aspect 37 or Aspect 38, wherein the disease is a metabolic disease.

Aspect 41 depends upon Aspect 37 or Aspect 38, wherein the subject has one or more symptoms of the disease.

Aspect 42 depends upon Aspect 37 or Aspect 38, wherein the subject does not have any symptoms of the disease.

Aspect 43 depends upon any one of Aspects 37 to 42, wherein the subject has been diagnosed with the disease.

Aspect 44 depends upon any one of Aspects 37 to 42, wherein the subject has not been diagnosed with the disease.

Aspect 45 depends upon any one of Aspects 37 to 44, wherein the subject has been previously treated for the disease.

Aspect 46 depends upon any one of Aspects 37 to 45, wherein the subject is administered an additional therapy.

Aspect 47 depends upon Aspect 46, wherein the additional therapy comprises radiotherapy, chemotherapy, or immunotherapy.

Aspect 48 includes a method for treating or preventing a disease indicated for an immunotherapy in a subject, the method comprising administering to the subject the antibody or antigen binding fragment of any one of Aspects 1 to 17, the polypeptide of any one of Aspects 18 to 21, the composition of any one of Aspects 22 to 26, or the host cell of Aspect 29 or Aspect 30.

Aspect 49 depends upon Aspect 48, wherein the subject is a human subject.

Aspect 50 depends upon Aspect 48 or Aspect 49, wherein the subject has one or more symptoms of the disease.

Aspect 51 depends upon Aspect 48 or Aspect 49, wherein the subject does not have one or more symptoms of the disease.

Aspect 52 depends upon Aspect 48 or Aspect 49, wherein the subject has been diagnosed with the disease.

Aspect 53 depends upon Aspect 48 or Aspect 49, wherein the subject has not been diagnosed with the disease.

Aspect 54 depends upon any one of Aspects 48 to 53, wherein the subject has been previously treated for the disease.

Aspect 55 depends upon Aspect 54, wherein the subject was resistant to the previous treatment.

Aspect 56 includes a method for evaluating a sample from a subject, the method comprising contacting a biological sample from the subject, or an extract thereof, with at least one antibody, antigen binding fragment, or polypeptide of any one of Aspects 1 to 21.

Aspect 57 depends upon Aspect 56, wherein the at least one antibody, antigen binding fragment, or polypeptide is operatively linked to a detectable label.

Aspect 58 depends upon Aspect 56 or Aspect 57, wherein the method further comprises incubating the antibody, antigen binding fragment, or polypeptide under conditions that allow for binding to antigens in the biological sample or extract thereof.

Aspect 59 depends upon any one of Aspects 56 to 58, wherein the method further comprises detecting the binding of an antigen to the antibody, antigen binding fragment, or polypeptide.

Aspect 60 depends upon any one of Aspects 56 to 59, wherein the method further comprises contacting the biological sample with at least one capture antibody, antigen, or polypeptide.

Aspect 61 depends upon Aspect 60, wherein the at least one capture antibody or fragment comprises at least one antibody or antigen binding fragment of any one of Aspects 1 to 15.

Aspect 62 depends upon Aspect 60 or Aspect 61, wherein the capture antibody or fragment is linked to a solid support.

Aspect 63 depends upon any one of Aspects 56 to 62, wherein the biological sample comprises a tissue sample or a blood sample.

Aspect 64 includes an antibody or antigen binding fragment comprising an LCDR1, LCDR2, LCDR3, HCDR1, HCDR2, and HCDR3 of clone R3A8 in Table 1.

Aspect 65 includes an antibody or antigen binding fragment comprising an LCDR1, LCDR2, LCDR3, HCDR1, HCDR2, and HCDR3 of clone R3E8 in Table 1.

Aspect 66 includes an antibody or antigen binding fragment comprising an LCDR1, LCDR2, LCDR3, HCDR1, HCDR2, and HCDR3 of clone R3E9 in Table 1.

Aspect 67 includes an antibody or antigen binding fragment comprising an LCDR1, LCDR2, LCDR3, HCDR1, HCDR2, and HCDR3 of clone R3E12 in Table 1.

Aspect 68 includes an antibody or antigen binding fragment comprising an LCDR1, LCDR2, LCDR3, HCDR1, HCDR2, and HCDR3 of clone R3H1 in Table 1.

Aspect 69 includes an antibody or antigen binding fragment comprising an LCDR1, LCDR2, LCDR3, HCDR1, HCDR2, and HCDR3 of clone R3H8 in Table 1.

Aspect 70 includes an antibody or antigen binding fragment comprising an LCDR1, LCDR2, LCDR3, HCDR1, HCDR2, and HCDR3 of clone R3G9 in Table 1.

Aspect 71 includes a method of treating a patient, the method comprising administering the antibody of any one of Aspects 64 to 70 to the patient.

Aspect 72 includes a method of treating a patient, the method comprising administering a pharmaceutical composition comprising the antibody of any one of Aspects 64 to 70 to the patient.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments or aspects described herein in the context of the term “comprising” may also be implemented in the context of the term “consisting of” or “consisting essentially of”

“Individual, “subject,” and “patient” are used interchangeably and can refer to a human or non-human.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Any embodiment or aspect discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1I: Generating sABs against a key mTORC1 functional site. FIG. 1A depicts Single-point ELISA sAB cross reactivity analysis using recombinant mTOR^{FRB} from common model organisms (n=3 independent replicates, mean±SD). FIG. 1B depicts binding kinetics determined by SPR for unique sABs engineered against mTOR^{FRB}. FIG. 1C depicts competitive single-point ELISA reveals overlapping epitopes between all unique sABs and the FKBP12-Rapamycin binding site of mTOR^{FRB} (n=3 independent replicates, mean±SD. **P≤0.01, ***P≤0.001, ****P≤0.0001, unpaired t-test). FIG. 1D depicts multi-point competitive ELISA screening for sAB-R3E9 and mTOR^{FRB} alone (black) or with 10 μM FKBP12-Rapamycin (white) in solution (n=3 independent replicates, mean±SD. ****P≤0.0001, one-way ANOVA). FIG. 1E depicts multi-point competitive ELISA screening for sAB-R3H8 and mTOR^{FRB} alone (black) or with 10 μM FKBP12-Rapamycin (white) in solution (n=3 independent replicates, mean±SD. ****P≤0.0001, one-way ANOVA). FIG. 1F depicts in vitro pulldown assay using 10 nM biotinylated SNAP-fused mTOR^{FRB} (Bio-mTOR^{FRB}), 10 nM Alexa488-labeled SNAP-fused FKBP12 (A488-FKBP12), 10 nM rapamycin, and varying concentrations of sAB-R3E9. Components were incubated for 30 minutes at room temperature before of Bio-mTOR^{FRB} was enriched via streptavidin magnetic beads. A488-FKBP12 levels were monitored by SDS-PAGE (n=3 independent replicates, mean±SD). FIG. 1G depicts in vitro pulldown assay using 10 nM biotinylated SNAP-fused mTOR^{FRB} (Bio-mTOR^{FRB}), 10 nM Alexa488-labeled SNAP-fused FKBP12 (A488-FKBP12), 10 nM rapamycin, and varying concentrations of sAB-R3H8. Components were incubated for 30 minutes at room temperature before Bio-mTOR^{FRB} was enriched via streptavidin magnetic beads. A488-FKBP12 levels were monitored by SDS-PAGE (n=3 independent replicates, mean±SD). FIG. 1H depicts representative immunoprecipitation-western blot analysis from Expi293F cells treated for 20 hours with vehicle or 1 μM Rapamycin. The indicated sABs were biotinylated through a C-terminal AviTag and added to lysates at 10 nM for 3 hours with rotation at 4° C. before enrichment with streptavidin magnetic beads. (n=3 biological replicates). FIG. 1I depicts representative immunoprecipitation-western blot analysis from Expi293F cells confirms that sAB-R3E9 and sAB-R3H8 can engage with and isolate mTORC1. The indicated sABs were biotinylated through a C-terminal AviTag and added to lysates at 50 nM for 3 hours with rotation at 4° C. before enrichment with streptavidin magnetic beads. (n=3 biological replicates).

FIGS. 2A-2H: a Structural features of sABs engaged with the mTOR^{FRB} substrate recruitment site. FIG. 2A depicts alignment of crystal structures for sAB-R3E9 (salmon) and sAB-R3H8 (pale cyan) bound to mTOR^{FRB}. FIG. 2B left depicts structural model of sAB-R3E9 and sAB-R3H8 in scFv format aligned with a previously published cryo-EM structure of mTORC1 (PDB: 6BCX). FIG. 2B right depicts close up view of mTOR^{FRB} and the active site (gray; ATP colored and shown as spheres) aligned with crystal structures of the S6K1³⁹²⁻⁴¹⁰-mTOR^{FRB} complex (yellow, PDB: 5WBH), FKBP12-Rapamycin-mTOR^{FRB} complex (orange, PDB: 1FAP), sAB-R3E9-mTOR^{FRB} (salmon, scFv format), and sAB-R3H8-mTOR^{FRB} (pale cyan, scFv format). FIG. 2C depicts quantification of buried solvent accessible surface area between mTOR^{FRB} and the indicated molecules. FIG. 2D depicts structural alignment of endogenous mTOR inhibitor PRAS40 (dark gray, PDB: 5WBU) and sAB-R3E9 (salmon) interactions with mTOR^{FRB} (light gray). Underlined amino acids indicate direct PRAS40-mTOR^{FRB} interactions. sAB-R3E9 residues that contact the same site are listed below PRAS40 residues. FIG. 2E depicts structural alignment of endogenous mTOR substrate S6K1 (yellow, PDB: 5WBH) and sAB-R3H8 (pale cyan) interactions with mTOR^{FRB} (light gray). Underlined amino acids indicate direct S6K1-mTOR^{FRB} interactions. sAB-R3H8 residues that contact the same site are listed below S6K1 residues. FIG. 2F depicts consensus hydrophobic interaction sites among endogenous (S6K1 and PRAS40) and synthetic (sAB-R3E9 and sAB-R3H8) structurally characterized mTOR^{FRB} interacting proteins. FIG. 2G depicts multipoint ELISA analysis for point mutations made in sAB-R3E9 at consensus interaction sites. (n=3 independent experiments, mean±SD). FIG. 2H depicts multipoint ELISA analysis for point mutations made in sAB-R3H8 at consensus interaction sites. (n=3 independent experiments, mean±SD).

FIGS. 3A-3L: Intrabody-based allosteric modulation of the mTOR-Raptor interaction. FIG. 3A depicts representative immunoprecipitation-western blot analysis of Expi293F cells transfected with empty vector (EV), isotype control (Iso), R3E9, or R3H8 FLAG-tagged intrabodies (Ib-FLAG) for 48 hours. FIG. 3B depicts quantification of mTOR enrichment from the FLAG immunoprecipitation relative to input shown in A. (n=3 biological replicates, mean±SD. ns=not significant, unpaired t-test). FIG. 3C depicts quantification of Raptor enrichment from the FLAG immunoprecipitation relative to input shown in A. (n=3 biological replicates, mean±SD. **P≤0.01, unpaired t-test). FIG. 3D depicts quantification of mLST8 enrichment from the FLAG immunoprecipitation relative to input shown in A. (n=3 biological replicates, mean±SD. ns=not significant, unpaired t-test). FIG. 3E depicts structure of sAB-R3E9 (dark gray) in single-chain variable fragment format with its epitope (salmon) illustrated on mTOR^{FRB} (light gray). FIG. 3F depicts structure of sAB-R3H8 (dark gray) in single-chain variable fragment format with its epitope (pale cyan) illustrated on mTOR^{FRB} (light gray). FIG. 3G depicts representative immunoprecipitation-western blot analysis for intrabody-based modulation of mTOR-Raptor and mTOR-Rictor interactions. Expi293F cells were transfected with empty vector (EV), isotype control (Iso), R3E9, or R3H8 FLAG-T2A-eGFP tagged intrabodies (Ib-FLAG-T2A-eGFP) for 24 hours before isolation of eGFP(+) cells by FACS. Vehicle or 200 nM Rapamycin were incubated with the indicated cells for 24 hours. Samples were then lysed and subjected to pan-mTOR immunoprecipitation for analysis of protein-protein interactions (n=3 biological replicates). FIG. 3H depicts 2mFo-DFc electron density map (1σ contour) after refinement for W2101 and S2035 of mTOR^{FRB} in the sAB-R3E9 bound state. FIG. 3I depicts 2mFo-DFc electron density map (1σ contour) after refinement for W2101 and S2035 of mTOR^{FRB} in the sAB-R3H8 bound state. FIG. 3J depicts sAB-R3E9 interactions (dark grey) in the hydrophobic groove 1 (HG1) of mTOR^{FRB} (salmon) illustrates stabilization of W2101 and S2035 side chains in unfavorable rotameric states. An extensive T-shaped and off-centered parallel displaced R-stacking network is formed between F93^{L3} and F108^{H3} of sAB-R3E9 and F2039, W2101, and F2108 of mTOR^{FRB}. FIG. 3K depicts sAB-R3H8 interactions (dark grey) in the hydrophobic groove 1 (HG1) of mTOR^{FRB} (pale cyan) illustrates favorable conformations of W2101 and S2035 side chains. FIG. 3L left depicts conformational change of mTOR^{FRB} in α3 and α4 helices in the R3E9 bound state (salmon) or R3H8 bound state (pale cyan). FIG. 3L center and right depicts alignment of α3 and α4 helices from mTOR^{FRB} in the R3E9 bound state (salmon), R3H8 bound state (pale cyan), and previously published FKBP12-Rapamycin bound state (PDB: 1NSG; light gray). Binding of R3E9 triggers subtle changes in the configuration of α3 and α4 helices compared to R3H8 or FKBP12-Rapamycin.

FIGS. 4A-4I: Genetically encoded inhibition of mTOR signaling with synthetic intrabodies in human cells. FIG. 4A depicts representative FACS isolation of Expi293F cells transfected with empty vector (EV), isotype control (Iso), R3E9, or R3H8 FLAG-T2A-eGFP tagged intrabodies (Ib-FLAG-T2A-eGFP) for 24 hours. FIG. 4B depicts representative western blot analysis of Expi293F cells transfected with empty vector (EV), isotype control (Iso), R3E9, or R3H8 FLAG-T2A-eGFP tagged intrabodies (Ib-FLAG-T2A-eGFP) for 24 hours before isolation of eGFP(+) cells by FACS. EV cells were treated with vehicle and rapamycin for 16 hours as controls. FIG. 4C depicts quantification of relative p-S6K1^{T389} levels from the experiment in B (n=3 biological replicates, mean±SD. ns=not significant, *P≤0.05, **P≤0.01, one-way ANOVA). FIG. 4D depicts representative western blot analysis of Expi293F cells transfected with the indicated constructs after 24-hour and 48-hour time points before isolation of eGFP(+) cells by FACS. FIG. 4E depicts quantification of relative p-S6K1^{T389} levels from the experiment in D (n=3 biological replicates, mean±SD. ****P≤0.0001, one-way ANOVA). FIG. 4F depicts quantification of relative p-Akt^{S473} levels from the experiment in D (n=3 biological replicates, mean±SD. *P≤0.01, ****P≤0.0001, one-way ANOVA). FIG. 4G depicts quantification of immunostained p-S6^{S240/244} levels from the experiment in H (n=19, 18, 21, 27, and 25 cells. ns=not significant, ****P≤0.0001, one-way ANOVA with Tukey-Kramer test). FIG. 4H depicts representative immunofluorescent confocal microscopy analysis of eGFP-tagged intrabody expression and of p-S6^{S240/244} levels in HeLa cells. Scale bar denotes 20.5 um. (n=2 independent experiments). FIG. 4I depicts schematic of differential mTORC1 and mTORC2 inhibition by Ib-R3E9 and Ib-R3H8 as determined by intracellular functional assays.

FIGS. 5A-5F: Subcellular compartment-specific inhibition of the endogenous mTOR signaling network. FIG. 5A depicts representative confocal microscopy analysis of eGFP-tagged Ib-R3E9 with the indicated subcellular localization tags in HeLa cells. Scale bar denotes 11.5 μm. FIG. 5B depicts schematic of subcellular mTOR activity modulation assay using cytosol- or nucleus-restricted eGFP-tagged intrabodies bearing either NES or NLS tags, respectively. FIG. 5C top depicts representative confocal microscopy analysis of the nuclear colocalization between H2A-mCherry-S6K1 (red) and Ib-R3E9-eGFP-NLSx2 (green) in HeLa cells. Scale bar denotes 9.2 μm. FIG. 5C bottom depicts fluorescence profile along the yellow line traced in the merged image above. FIG. 5D depicts Western blot analysis of HeLa cells transfected with empty vector (EV) controls or co-transfected with H2A-mCherry-S6K1 and isotype control (Iso), R3E9, or R3H8 intrabodies bearing eGFP-NLSx2 tags for 48 hours before treatment with vehicle or 500 nM Rapamycin for 1 hour. FIG. 5E depicts quantification of p-T389 H2A-mChe-S6K1 relative to total H2A-mChe-S6K1 and p-T389 S6K1 relative to actin from the experiment in FIG. 5D. Label of n.d. indicates no data collected for empty vector controls. (n=3 biological replicates, mean±SD. ns=not significant, ****P≤0.0001, one-way ANOVA). FIG. 5F depicts representative confocal microscopy analysis of endogenous mTOR signaling in HeLa cells transfected with eGFP-NESx2- or eGFP-NLSx2-tagged isotype control (Iso), R3E9, or R3H8 intrabodies. Transfected cells were immunostained to determine endogenous levels of p-S6^{S240/244} in the presence of cytosol- or nucleus-restricted intrabodies. Scale bar denotes 15.4 μm. Graphs on the right report fluorescence intensity profiles along the traced yellow lines shown in merged images. (n=2 independent experiments).

FIGS. 6A-6D: Selection of synthetic antibody fragments and conversion to single-chain variable fragment intrabodies. FIG. 6A depicts domain map of mTOR with FRB highlighted as the target used for epitope-directed phage display. FIG. 6B depicts representative phage ELISA screening for unique clones resulting from five rounds of phage display biopanning. FIG. 6C depicts construct design for single-chain variable fragment intrabodies with C-terminal FLAG tag. FIG. 6D depicts Western blot analysis of Expi293F cells that were transfected with the indicated intrabodies for 48 hours before harvesting. Values indicate relative intrabody expression levels normalized to R3E9.

FIGS. 7A-7B: Biophysical characterization of sABs. FIG. 7A depicts multi-point SPR kinetics for sAB binding to immobilized mTOR^{FRB}. sABs were run as analytes in two-fold serial dilutions. Curve fitting was performed using a 1:1 binding model. FIG. 7B depicts multi-point competitive ELISA screening for mTOR^{FRB} alone (black) or with 10 μM FKBP12-Rapamycin (white) in solution for the indicated sABs (n=3, mean±SD).

FIGS. 8A-8C: In vitro pulldown assay for competition of sABs with FKBP12-Rapamycin. FIG. 8A depicts schematic of the fluorescence-based in vitro streptavidin bead pulldown assay. FIG. 8B depicts representative assessment of the assay using 10 nM biotinylated SNAP-mTOR^{FRB} and 10 nM Alexa488-SNAP-FKBP12 incubated with the indicated concentrations of rapamycin. FIG. 8C depicts representative SDS-PAGE readouts for Alexa488 fluorescence from in vitro pulldowns using titrations of sAB-R3E9 (top) and sAB-R3H8 (bottom).

FIG. 9: Left depicts immunofluorescent co-staining of HeLa cells with sAB-R3E9 (red) and an anti-LAMP1 (green) antibody. Right depicts line trace profile from the line drawn in the merged image.

FIGS. 10A-10C: Detailed analysis of interactions at the mTOR^{FRB} substrate recruitment site. FIG. 10A depicts detailed CDR interactions of sAB-R3E9 (left) and sAB-R3H8 (right) with mTOR^{FRB}. FIG. 10B depicts analysis of mTOR^{FRB} residue solvent accessible surface area burial by sAB-R3E9 compared to a previously published structure of mTOR^{FRB} bound by FKBP12-Rapamycin (PDB: 1FAP). FIG. 10C depicts multiple sequence alignment of mTOR and contacts made by the indicated molecules above. Black circles represent interaction of the indicated molecule with with H. sapiens mTOR^{FRB}. Uniprot IDs from top to bottom: P42345, Q9JLN9, Q9VK45, Q95Q95, P35169, P32600.

FIGS. 11A-11B: Alanine scanning mutagenesis of consensus hydrophobic interaction sites within the mTORFRB substrate recruitment interface. FIG. 11A top depicts EC50 values (nM) for alanine mutations made in sAB-R3E9. FIG. 11A bottom depicts visualization of the fold change in EC50 values for sAB-R3E9 alanine mutations (n=3 independent experiments, mean±SD). FIG. 11B top depicts EC50 values (nM) for alanine mutations made in sAB-R3H8. FIG. 11B bottom depicts visualization of the fold change in EC50 values for sABR3H8 alanine mutations. F110A EC50 value could not be determined (n.d.) (n=3 independent experiments, mean±SD).

FIG. 12: Representative pan-mTOR co-immunoprecipitation western blot analysis from Expi293F cell lysates alone or lysates spiked with biotinylated sABs. Briefly, cells were treated with vehicle or 200 nM Rapamycin for 24 hours. Vehicle treated lysate was split and spiked with biotinylated isotype control (Iso), biotinylated sAB-R3E9, or biotinylated sAB-R3H8 and incubated at 4 C before performing pan-mTOR immunoprecipitation for western blot analysis.

FIGS. 13A-13F: Structural comparison of sAB-R3E9, sAB-R3H8, and rapamycin. FIG. 13A depicts binding pose of rapamycin from the previously determined FKBP12-Rapamycin-mTOR^{FRB} crystal structure (PDB: 1FAP). FIG. 13B depicts sAB-R3E9 residues with similar spatial arrangement as rapamycin. FIG. 13C depicts sAB-R3H8 residues with similar spatial arrangement as rapamycin. FIG. 13D depicts mTOR^{FRB} surface electrostatics when bound to FKBP12-Rapamycin (PDB: 1FAP). FIG. 13E depicts mTOR^{FRB} surface electrostatics when bound to sAB-R3E9. Hydrophobic grooves 1 and 2 (HG1 and HG2) marked by arrows. FIG. 13F depicts mTOR^{FRB} surface electrostatics when bound to sAB-R3H8. Epitopes of the indicated molecules are traced with black dashed line.

FIG. 14: View of W2101 and 52035 (circled) interactions in hydrophobic groove 1 (HG1) from previously published structures of mTOR^{FRB} bound by FKBP12-Rapamycin (PDB 1FAP; some rapamycin atoms omitted for clarity of 52035 represented by dashed line), S6K1 (PDB 5WBH), and PRAS40 (PDB 5WBU). These interactions are compared to sAB-R3E9 bound and sAB-R3H8 bound structures, illustrating the novel conformation of mTOR^{FRB} stabilized by sAB-R3E9.

FIG. 15: Representative intrabody immunoprecipitation-western blot analysis of Expi293F cells transfected with empty vector (EV), isotype control (Iso), R3E9, or R3H8 FLAG-tagged intrabodies (Ib-FLAG) for 48 hours.

FIGS. 16A-16D: Comparison of intrabody-based mTOR modulation to rapamycin. FIG. 16A depicts relative quantification of total mTOR protein expression from eGFP(+) cells isolated by FACS used in the western blot in FIG. 4B. (n=3 biological replicates, mean±SD. ns=not significant, one-way ANOVA). FIG. 16B depicts representative western blot analysis of Expi293F cells treated with vehicle or 100 nM rapamycin for the indicated time points. Samples are from the same membrane but irrelevant samples in between vehicle and rapamycin treatments have been cropped from this image. FIG. 16C depicts quantification of S6K1 phosphorylation from A. (n=2 biological replicates SD. ***P≤0.001, one-way ANOVA). FIG. 16D depicts quantification of Akt phosphorylation from A. (n=2 biological replicates, mean±SD. **P≤0.01, ***P≤0.001, one-way ANOVA).

FIGS. 17A-17B: Directing the expression of intrabodies to selected subcellular locations. FIG. 17A depicts schematic of eGFP-tagged intrabody constructs bearing the indicated subcellular localization tags. FIG. 17B left depicts representative confocal microscopy analysis of the nuclear colocalization of H2A-mCherry-S6K1 (red) and Ib-R3H8-eGFP-NLSx2 (green) in HeLa cells. Scale bar denotes 9.2 μm. FIG. 17B right depicts line trace quantification of fluorescence intensity along the yellow line drawn in the merged image on left.

### DETAILED DESCRIPTION OF THE INVENTION

The synthetic protein-based inhibitors of mTOR described in certain aspects herein are fully genetically encodable in living cells. By fusing scFvs with subcellular localization tags, intracellular scFv expression can be spatially restricted to specific cellular locations, including the plasma membrane, cytosol, and nucleus. Leveraging spatial control over intracellular scFv expression can enable ones skilled in the art to systematically explore or perturb the subcellular roles that mTOR plays in noncanonical cellular locations.

Subcellular compartmentalization is integral to the spatial regulation of mechanistic target of rapamycin (mTOR) signaling. However, the biological outputs associated with location-specific mTOR signaling events are poorly understood and challenging to decouple. Aspects herein include synthetic intracellular antibodies (which can be referred to as intrabodies) that are capable of modulating mTOR signaling with genetically programmable spatial resolution. Epitope-directed phage display is exploited in certain aspects to generate high affinity synthetic antibody fragments (Fabs) against the FKBP12-Rapamycin binding site of mTOR (mTORFRB). Aspects herein include high resolution crystal structures of two unique Fabs that discriminate distinct conformational states of mTORFRB through recognition of its substrate recruitment interface. By leveraging these conformation-specific binders as intracellular probes, aspects herein show the structural basis for an allosteric mechanism governing mTOR complex 1 (mTORC1) stability mediated by subtle structural adjustments within mTORFRB. Furthermore, aspects herein provide synthetic binders that emulate natural substrates by employing flexible yet complementary hydrophobic residues at defined positions, underscoring the propensity of mTORFRB to coordinate diverse molecular interfaces. Intracellular signaling studies showed differential time-dependent inhibition of S6K1 and Akt phosphorylation by genetically encoded intrabodies, thus supporting a mechanism of inhibition analogous to the natural product rapamycin. Aspects herein also provide a feasible approach to selectively modulate mTOR signaling in the nucleus through spatially programmed intrabody expression. Certain aspects establish intrabodies as versatile tools for dissecting the conformational regulation of mTORC1 and such intrabodies can be useful to explore how location-specific mTOR signaling influences disease progression.

To futher elaborate, elucidating subcellular mechanisms of protein kinase signaling has been challenging in the art due to the lack of spatial precision by genetic knockdown or pharmacological approaches. Accordingly certain synthetic intracellular antibodies (intrabodies) described herein are engineered as genetically encodable inhibitors of mechanistic target of rapamycin (mTOR) signaling. The inventors demonstrate the versatility of intrabodies as conformational probes by uncovering an allosteric mechanism that regulates the stability of mTOR complex 1. Furthermore, the inventors illustrate the feasibility of decoupling subcellular spatial mTOR networks through direct functional perturbations by cytosol- or nucleus-restricted intrabody expression. This work highlights the potential of synthetic intrabodies for investigating structural and spatial mechanisms of therapeutically relevant protein kinase networks.

The current repertoire of mTOR inhibitors operates on a whole-cell basis by disrupting catalysis, substrate recruitment, or both (21, 35, 36). The inventors envisioned that recapitulating the molecular mechanism of rapamycin, which directly blocks substrate recruitment, using intracellular antibodies (intrabodies) would enable the inventors to capture its therapeutic profile while maintaining control over its location inside cells (37). Rapamycin inhibits mTOR by first forming a complex with FK506-binding protein of 12 kDa (FKBP12) and then sequestering the FKBP12-Rapamycin binding (FRB) domain of mTOR (mTOR^{FRB}) located next to the catalytic cleft (38). This interaction selectively inhibits the phosphorylation of specific mTOR substrates, though not all, owing to the mechanistic diversity underlying mTOR substrate recruitment (39-41). Structural studies demonstrated that mTOR^{FRB} docking of ribosomal protein S6 kinase 1 (S6K1) and proline-rich Akt substrate of 40 kDa (PRAS40) is a competitive mechanism for regulating mTORC1 activity and suggested that several other mTORC1 substrates may require mTOR^{FRB} docking for their phosphorylation as well (42, 43). Therefore, the inventors exploited the power of phage display to generate epitope-directed synthetic antibody fragments (Fabs) (44, 45) against the rapamycin binding site as a straightforward route to modulate mTOR signaling genetically. Synthetic affinity reagents provide distinctive advantages over traditional gene knockdowns or small molecule inhibitors, including feasible tuning of target activity, masking of functional sites, and spatiotemporal control (46-50). Furthermore, Fabs serve as superior crystallization chaperones (51), and conversion to single chain variable fragment (scFv) format enables simple genetic portability into living cells (52).

### I. ANTIBODIES

Aspects of the disclosure relate to antibodies, antigen binding fragments thereof, or polypeptides capable of specifically binding to mTOR, including a FKBP12-Rapamycin binding domain of mTOR.

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

### A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1 (or LCDR1), CDR-L2 (or LCDR2), and CDR-L3 (or LCDR3). The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1 (or HCDR1), CDR-H2 (or HCDR2), and CDR-H3 (or HCDR3). The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:
1) Computational predictions of the tertiary structure of the antibody/epitope binding interactions based on the chemical nature of the amino acid sequence of the antibody variable region and composition of the epitope.
2) Hydrogen-deuterium exchange and mass spectroscopy
3) Polypeptide fragmentation and peptide mapping approaches in which one generates multiple overlapping peptide fragments from the full length of the polypeptide and evaluates the binding affinity of these peptides for the epitope.
4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### B. Functional Antibody Fragments and Antigen-Binding Fragments

### 1. Antigen-Binding Fragments

Certain aspects relate to antibody fragments, such as antibody fragments that bind to mTOR, including a FKBP12-Rapamycin binding domain of mTOR. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment (also “Fab” or “FAB”) means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### 2. Fragment Antigen Binding Region, Fab

Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature.

### 3. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

C. Polypeptides with Antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is ≤5×10⁻¹⁰ M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

### E. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the invention to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### II. ANTIBODY PRODUCTION

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Other methods of making antibodies are through molecular display technologies. These include technologies such as phage display and yeast surface display, have been the major driving force of the development (Winter, 1994; Boder, 2000; Viti, 2000; Bradbury, 2011). These enable display of single-chain Fv (scFv) and Fab fragments on the surface of microorganisms and links the phenotype of a variant to its cognate DNA and thus amino acid sequence (McCafferty, 1990; Kang, 1991; Barbas, 1991). Using these techniques, it is possible to produce a library of an antibody fragment that contains billions of variants and identify variants with a desired binding function from the library in a HTP manner.

In the standard antibody architecture, six surface loops, three each from the heavychain and light-chain variable domains constitute the complementarity determining regions (CDRs). Thus, the antibody and its fragments can be considered as “molecular scaffolds” for presenting CDRs. The sequence diversity of CDRs is the primary source of antibody affinity and specificity. In recombinant antibody libraries, the diversity of CDRs can be either derived from natural immune systems or generated by random mutagenesis, and Fv libraries of both types have been successfully used (Barbas, 1992; Knappik, 2000).

Yeast surface display is another important technical development in antibody engineering. Although the library size achievable with this technique is much smaller than that with phage display, yeast surface display coupled with fluorescence activated cell sorter (FACS) allows for HTP, quantitative characterization of antibody affinity, specificity and expression efficiency (Boder, 2000). The major strengths of phage display include the large size of a library and rapid completion of the selection and propagation cycle (<2 days). Yeast display provides a highly quantitative readout of binding and expression levels using FACS and the ability to precisely define the selection stringency based on equilibrium and kinetic binding parameters as well as expression levels. The integration of these two display methods can provide for an optimal selection of clones that not only are high affinity binders but are stable and express well.

The end products of recombinant antibody screening are the genes encoding Fv and Fab fragments with desired binding characteristics. Bacterial expression systems for Fv and Fab with good yields have been established [Borrebaeck, 1992]. Thus, the phage display clones can be quickly reformatted into an expression cassette for the production of soluble Fv and Fab fragments.

Library design: At least 15-20 residues of CDR residues contribute to antibody binding to its target. Full combinatorial randomization of this many sites together far exceeds the limits of phage and yeast display library diversity. Since the number of sites cannot be significantly reduced, new library strategies have been developed that reduce the 20 natural amino acid genetic code to a smaller subset, while maintaining high functional diversity (Fellouse, 2007). These libraries using a greatly reduced genetic code can produce high affinity Fab and scFv molecules. Although several reduced genetic code libraries have been constructed and tested, the concept has not been optimized for different target classes and especially for the smaller scaffold camelids VHH and FN3 domains (D2). For screening of these novel libraries, the inventors will combine phage display and yeast surface display for optimal efficiency. Humanized antibody scaffolds—In molecular display approaches only the variable regions of the antigen binding loops (CDRs) of the Fab domain are generally diversified. The main architecture of the domain is held constant. This main architecture can be based on a fully human antibody scaffold. This circumvents the need to humanize the scaffold, as is required by methods involving animal immunizations.

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as 0-interferon, IL-2, or IL-12, or genes encoding proteins involved in immune helper functions, such as B-7.A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

### III. ANTIBODIES, ANTIGEN BINDING FRAGMENTS, AND POLYPEPTIDES

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. The term polypeptide also includes an antibody fragment described herein as well as antibody domains, such as HCDR1 (Heavy chain complementarity determing region 1), HCDR2 (Heavy chain complementarity determing region 2), HCDR3 (Heavy chain complementarity determing region 3), LCDR1 (Light chain complementarity determing region 1), LCDR2 (Light chain complementarity determing region 2), LCDR3 (Light chain complementarity determing region 3), HFRW1 (Heavy chain framework region 1), HFRW2 (Heavy chain framework region 2), HFRW3 (Heavy chain framework region 3), HFRW4 (Heavy chain framework region 4), LFRW1 (Light chain framework region 1), LFRW2 (Light chain framework region 21), LFRW3 (Light chain framework region 3), LFRW4 (Light chain framework region 4), VH (Heavy chain variable region), VL (Light chain variable region), CH (Heavy chain constant region), or CL (Light chain constant region).

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of an antibody, antigen binding fragment, protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The antibody, antigen binding fragment, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of a polypeptide of Table 1.

In some embodiments, the antibody, antigen binding fragment, protein, or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112,113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of a polypeptide of Table 1.

In some embodiments, the antibody, antigen binding fragment, or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of a polypeptide of Table 1.

In some embodiments, the antibody, antigen binding fragment, protein, or polypeptide may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of a polypeptide of Table 1 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of a polypeptide of Table 1.

In some aspects there is a nucleic acid molecule, antibody, antigen binding fragment, protein, or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any polypeptide of Table 1 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any polypeptide of Table 1.

In some embodiments, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the heavy chain, light chain, VH, VL, HCDR1, HCDR2, HCDR3, LCDR1, LCDR2, LCDR3, HFRW1, HFRW2, HFRW3, HFRW4, LFRW1, LFRW2, LFRW3, or LFRW4 identified in Table 1 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some embodiments, a polypeptide (e.g., antibody, antibody fragment, Fab, etc.) of the disclosure comprises a CDR that is at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 10000 identical (or any range derivable therein) in sequence to one polypeptide of Table 1. In some embodiments, a polypeptide comprises 1, 2, and/or 3 CDRs from one of SEQ ID NOs:1-502. The CDR may be one that has been determined by Kabat, IMGT, or Chothia. In further embodiments, a polypeptide may have CDRs that have 1, 2, and/or 3 amino acid changes (e.g., addition of 1 or 2 amino acids, deletions of 1 or 2 amino acids, substitution) with respect to these 1, 2, or 3 CDRs. In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

From amino to carboxy terminus the CDRs are CDR1, CDR2, and CDR3. In some embodiments, a polypeptide may have CDRs that have 1, 2, and/or 3 amino acid changes (e.g., addition of 1 or 2 amino acids, deletions of 1 or 2 amino acids, substitution) with respect to CDR1, CDR2, or CDR3. In some embodiments, the CDRs of Table 1 may further comprise 1, 2, 3, 4, 5, or 6 additional amino acids at the amino or carboxy terminus of the CDR, The additional amino acids may be from the heavy and/or light chain framework regions of Table 1, that are shown as immediately adjacent to the CDRs. Accordingly, embodiments relate to polypeptides comprising an HCDR1 (i.e., CDR-H1), HCDR2 (i.e., CDR-H2), HCDR3 (i.e., CDR-H3), LCDR1 (i.e., CDR-L1), LCDR2 (i.e., CDR-L2), and/or LCDR3 (i.e., CDR-L3) with at least or at most or exactly 1, 2, 3, 4, 5, 6 or 7 amino acids at the amino end of the CDR or at the carboxy end of the CDR, wherein the additional amino acids are the 1, 2, 3, 4, 5, 6, or 7 amino acids of Table 1 that are shown as immediately adjacent to the CDRs. Other embodiments relate to antibodies comprising one or more CDRs, wherein the CDR is a fragment of Table 1 and wherein the fragment lacks 1, 2, 3, 4, or 5 amino acids from the amino or carboxy end of the CDR. In some embodiments, the CDR may lack one, 2, 3, 4, 5, 6, or 7 amino acids from the carboxy end and may further comprise 1, 2, 3, 4, 5, 6, 7, or 8 amino acids from the framework region of the amino end of the CDR. In some embodiments, the CDR may lack one, 2, 3, 4, 5, 6, or 7 amino acids from the amino end and may further comprise 1, 2, 3, 4, 5, 6, 7, or 8 amino acids from the framework region of the carboxy end of the CDR. In further embodiments, an antibody may be alternatively or additionally humanized in regions outside the CDR(s) and/or variable region(s). In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

In other embodiments, a polypeptide or protein comprises 1, 2, 3, 4, 5, or 6 CDRs from either or both of the light and heavy variable regions of Table 1, and 1, 2, 3, 4, 5, or 6 CDRs may have 1, 2, and/or 3 amino acid changes with respect to these CDRs. In some embodiments, parts or all of the antibody sequence outside the variable region have been humanized. A protein may comprise one or more polypeptides. In some aspects, a protein may contain one or two polypeptides similar to a heavy chain polypeptide and/or 1 or 2 polypeptides similar to a light chain polypeptide.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

TABLE 1
Antibody and Antigen Binding Domain Sequences

| | | | SEQ ID |
|---|---|---|---|
| Clone | Description | Sequence | NO: |
| R3A8 | HCDR1 | FYSSYI | 10 |
| | HCDR2 | SISSSSGSTS | 11 |
| | HCDR3 | FSSYYYTLYWAM | 12 |
| | LCDR1 | SVSSA | 13 |
| | LCDR2 | SASSLYS | 14 |
| | LCDR3 | SIHYPL | 15 |
| | VH | EISEVQLVESGGGLVQPGGSLRLSCAASGFN | 16 |
| | | FYSSYIHWVRQAPGKGLEWVASISSSSGSTS | |
| | | YADSVKGRFTISADTSKNTAYLQMNSLRAE | |
| | | DTAVYYCARFSSYYYTLYWAMDYWGQGTL | |
| | | VTV | |
| | VL | SDIQMTQSPSSLSASVGDRVTITCRASQSVSS | 17 |
| | | AVAWYQQKPGKAPKLLIYSASSLYSGVPSRF | |
| | | SGSRSGTDFTLTISSLQPEDFATYYCQQSIHY | |
| | | PLTFGQGTKVEIKRT | |
| | Full heavy chain | EISEVQLVESGGGLVQPGGSLRLSCAASGFN | 18 |
| | | FYSSYIHWVRQAPGKGLEWVASISSSSGSTS | |
| | | YADSVKGRFTISADTSKNTAYLQMNSLRAE | |
| | | DTAVYYCARFSSYYYTLYWAMDYWGQGTL | |
| | | VTVSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNHKP | |
| | | SNTKVDKKVEPKSCDKTHT | |
| | Full light chain | SDIQMTQSPSSLSASVGDRVTITCRASQSVSS | 19 |
| | | AVAWYQQKPGKAPKLLIYSASSLYSGVPSRF | |
| | | SGSRSGTDFTLTISSLQPEDFATYYCQQSIHY | |
| | | PLTFGQGTKVEIKRTVAAPSVFIFPPSDSQLK | |
| | | SGTASVVCLLNNFYPREAKVQWKVDNALQS | |
| | | GNSQESVTEQDSKDSTYSLSSTLTLSKADYE | |
| | | KHKVYACEVTHQGLSSPVTKSFNRGEC | |
| | Single chain | SDIQMTQSPSSLSASVGDRVTITCRASQSVSS | 20 |
| | variable fragment | AVAWYQQKPGKAPKLLIYSASSLYSGVPSRF | |
| | (VL-VH) | SGSRSGTDFTLTISSLQPEDFATYYCQQSIHY | |
| | | PLTFGQGTKVEIKGTTAASGSSGGSSSGASSS | |
| | | EVQLVESGGGLVQPGGSLRLSCAASGFNFYS | |
| | | SYIHWVRQAPGKGLEWVASISSSSGSTSYAD | |
| | | SVKGRFTISADTSKNTAYLQMNSLRAEDTAV | |
| | | YYCARFSSYYYTLYWAMDYWGQGTLVTVS | |
| | | SA | |
| R3E8 | HCDR1 | FSSSYI | 21 |
| | HCDR2 | SISSSSSSTS | 22 |
| | HCDR3 | YVYSYSQGGPAF | 23 |
| | LCDR1 | SVSSA | 24 |
| | LCDR2 | SASSLYS | 25 |
| | LCDR3 | SWSVPI | 26 |
| | VH | EISEVQLVESGGGLVQPGGSLRLSCAASGFN | 27 |
| | | FSSSYIHWVRQAPGKGLEWVASISSSSSSTSY | |
| | | ADSVKGRFTISADTSKNTAYLQMNSLRAEDT | |
| | | AVYYCARYVYSYSQGGPAFDYWGQGTLVT | |
| | | V | |
| | VL | SDIQMTQSPSSLSASVGDRVTITCRASQSVSS | 28 |
| | | AVAWYQQKPGKAPKLLIYSASSLYSGVPSRF | |
| | | SGSRSGTDFTLTISSLQPEDFATYYCQQSWSV | |
| | | PITFGQGTKVEIKRT | |
| | Full heavy chain | EISEVQLVESGGGLVQPGGSLRLSCAASGFN | 29 |
| | | FSSSYIHWVRQAPGKGLEWVASISSSSSSTSY | |
| | | ADSVKGRFTISADTSKNTAYLQMNSLRAEDT | |
| | | AVYYCARYVYSYSQGGPAFDYWGQGTLVT | |
| | | VSSASTKGPSVFPLAPSSKSTSGGTAALGCLV | |
| | | KDYFPEPVTVSWNSGALTSGVHTFPAVLQSS | |
| | | GLYSLSSVVTVPSSSLGTQTYICNVNHKPSNT | |
| | | KVDKKVEPKSCDKTHT | |
| | Full light chain | SDIQMTQSPSSLSASVGDRVTITCRASQSVSS | 30 |
| | | AVAWYQQKPGKAPKLLIYSASSLYSGVPSRF | |
| | | SGSRSGTDFTLTISSLQPEDFATYYCQQSWSV | |
| | | PITFGQGTKVEIKRTVAAPSVFIFPPSDSQLKS | |
| | | GTASVVCLLNNFYPREAKVQWKVDNALQS | |
| | | GNSQESVTEQDSKDSTYSLSSTLTLSKADYE | |
| | | KHKVYACEVTHQGLSSPVTKSFNRGEC | |
| | Single chain | SDIQMTQSPSSLSASVGDRVTITCRASQSVSS | 31 |
| | variable fragment | AVAWYQQKPGKAPKLLIYSASSLYSGVPSRF | |
| | (VL-VH) | SGSRSGTDFTLTISSLQPEDFATYYCQQSWSV | |
| | | PITFGQGTKVEIKGTTAASGSSGGSSSGASSS | |
| | | EVQLVESGGGLVQPGGSLRLSCAASGFNFSS | |
| | | SYIHWVRQAPGKGLEWVASISSSSSSTSYAD | |
| | | SVKGRFTISADTSKNTAYLQMNSLRAEDTAV | |
| | | YYCARYVYSYSQGGPAFDYWGQGTLVTVSS | |
| | | A | |
| R3E9 | HCDR1 | VYSYSI | 32 |
| | HCDR2 | SIYPSSGYTS | 33 |
| | HCDR3 | GTYYFYFMSWGL | 34 |
| | LCDR1 | SVSSA | 35 |
| | LCDR2 | SASSLYS | 36 |
| | LCDR3 | YFSSLF | 37 |
| | VH | EISEVQLVESGGGLVQPGGSLRLSCAASGFN | 38 |
| | | VYSYSIHWVRQAPGKGLEWVASIYPSSGYTS | |
| | | YADSVKGRFTISADTSKNTAYLQMNSLRAE | |
| | | DTAVYYCARGTYYFYFMSWGLDYWGQGTL | |
| | | VTV | |
| | VL | SDIQMTQSPSSLSASVGDRVTITCRASQSVSS | 39 |
| | | AVAWYQQKPGKAPKLLIYSASSLYSGVPSRF | |
| | | SGSRSGTDFTLTISSLQPEDFATYYCQQYFSS | |
| | | LFTFGQGTKVEIKRT | |
| | Full heavy chain | EISEVQLVESGGGLVQPGGSLRLSCAASGFN | 40 |
| | | VYSYSIHWVRQAPGKGLEWVASIYPSSGYTS | |
| | | YADSVKGRFTISADTSKNTAYLQMNSLRAE | |
| | | DTAVYYCARGTYYFYFMSWGLDYWGQGTL | |
| | | VTVSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNHKP | |
| | | SNTKVDKKVEPKSCDKTHT | |
| | Full light chain | SDIQMTQSPSSLSASVGDRVTITCRASQSVSS | 41 |
| | | AVAWYQQKPGKAPKLLIYSASSLYSGVPSRF | |
| | | SGSRSGTDFTLTISSLQPEDFATYYCQQYFSS | |
| | | LFTFGQGTKVEIKRTVAAPSVFIFPPSDSQLK | |
| | | SGTASVVCLLNNFYPREAKVQWKVDNALQS | |
| | | GNSQESVTEQDSKDSTYSLSSTLTLSKADYE | |
| | | KHKVYACEVTHQGLSSPVTKSFNRGEC | |
| | Single chain | SDIQMTQSPSSLSASVGDRVTITCRASQSVSS | 42 |
| | variable fragment | AVAWYQQKPGKAPKLLIYSASSLYSGVPSRF | |
| | (VL-VH) | SGSRSGTDFTLTISSLQPEDFATYYCQQYFSS | |
| | | LFTFGQGTKVEIKGTTAASGSSGGSSSGASSS | |
| | | EVQLVESGGGLVQPGGSLRLSCAASGFNVYS | |
| | | YSIHWVRQAPGKGLEWVASIYPSSGYTSYA | |
| | | DSVKGRFTISADTSKNTAYLQMNSLRAEDTA | |
| | | VYYCARGTYYFYFMSWGLDYWGQGTLVTV | |
| | | SSA | |
| R3E12 | HCDR1 | VYSSSI | 43 |
| | HCDR2 | SIYSYSSSTS | 44 |
| | HCDR3 | FDWYQRYSFYEAM | 45 |
| | LCDR1 | SVSSA | 46 |
| | LCDR2 | SASSLYS | 47 |
| | LCDR3 | SYFYPV | 48 |
| | VH | EISEVQLVESGGGLVQPGGSLRLSCAASGFN | 49 |
| | | VYSSSIHWVRQAPGKGLEWVASIYSYSSSTS | |
| | | YADSVKGRFTISADTSKNTAYLQMNSLRAE | |
| | | DTAVYYCARFDWYQRYSFYEAMDYWGQG | |
| | | TLVTV | |
| | VL | SDIQMTQSPSSLSASVGDRVTITCRASQSVSS | 50 |
| | | AVAWYQQKPGKAPKLLIYSASSLYSGVPSRF | |
| | | SGSRSGTDFTLTISSLQPEDFATYYCQQSYFY | |
| | | PVTFGQGTKVEIKRT | |
| | Full heavy chain | EISEVQLVESGGGLVQPGGSLRLSCAASGFN | 51 |
| | | VYSSSIHWVRQAPGKGLEWVASIYSYSSSTS | |
| | | YADSVKGRFTISADTSKNTAYLQMNSLRAE | |
| | | DTAVYYCARFDWYQRYSFYEAMDYWGQG | |
| | | TLVTVSSASTKGPSVFPLAPSSKSTSGGTAAL | |
| | | GCLVKDYFPEPVTVSWNSGALTSGVHTFPA | |
| | | VLQSSGLYSLSSVVTVPSSSLGTQTYICNVNH | |
| | | KPSNTKVDKKVEPKSCDKTHT | |
| | Full light chain | SDIQMTQSPSSLSASVGDRVTITCRASQSVSS | 52 |
| | | AVAWYQQKPGKAPKLLIYSASSLYSGVPSRF | |
| | | SGSRSGTDFTLTISSLQPEDFATYYCQQSYFY | |
| | | PVTFGQGTKVEIKRTVAAPSVFIFPPSDSQLK | |
| | | SGTASVVCLLNNFYPREAKVQWKVDNALQS | |
| | | GNSQESVTEQDSKDSTYSLSSTLTLSKADYE | |
| | | KHKVYACEVTHQGLSSPVTKSFNRGEC | |
| | Single chain | SDIQMTQSPSSLSASVGDRVTITCRASQSVSS | 53 |
| | variable fragment | AVAWYQQKPGKAPKLLIYSASSLYSGVPSRF | |
| | (VL-VH) | SGSRSGTDFTLTISSLQPEDFATYYCQQSYFY | |
| | | PVTFGQGTKVEIKGTTAASGSSGGSSSGASSS | |
| | | EVQLVESGGGLVQPGGSLRLSCAASGFNVYS | |
| | | SSIHWVRQAPGKGLEWVASIYSYSSSTSYAD | |
| | | SVKGRFTISADTSKNTAYLQMNSLRAEDTAV | |
| | | YYCARFDWYQRYSFYEAMDYWGQGTLVTV | |
| | | SSA | |
| R3H1 | HCDR1 | VSSSYI | 54 |
| | HCDR2 | YIYPYSGYTS | 55 |
| | HCDR3 | NYGGYPRKYAGAF | 56 |
| | LCDR1 | SVSSA | 57 |
| | LCDR2 | SASSLYS | 58 |
| | LCDR3 | SYGSLI | 59 |
| | VH | EISEVQLVESGGGLVQPGGSLRLSCAASGFN | 60 |
| | | VSSSYIHWVRQAPGKGLEWVAYIYPYSGYT | |
| | | SYADSVKGRFTISADTSKNTAYLQMNSLRAE | |
| | | DTAVYYCARNYGGYPRKYAGAFDYWGQGT | |
| | | LVTV | |
| | VL | SDIQMTQSPSSLSASVGDRVTITCRASQSVSS | 61 |
| | | AVAWYQQKPGKAPKLLIYSASSLYSGVPSRF | |
| | | SGSRSGTDFTLTISSLQPEDFATYYCQQSYGS | |
| | | LITFGQGTKVEIKRT | |
| | Full heavy chain | EISEVQLVESGGGLVQPGGSLRLSCAASGFN | 62 |
| | | VSSSYIHWVRQAPGKGLEWVAYIYPYSGYT | |
| | | SYADSVKGRFTISADTSKNTAYLQMNSLRAE | |
| | | DTAVYYCARNYGGYPRKYAGAFDYWGQGT | |
| | | LVTVSSASTKGPSVFPLAPSSKSTSGGTAALG | |
| | | CLVKDYFPEPVTVSWNSGALTSGVHTFPAVL | |
| | | QSSGLYSLSSVVTVPSSSLGTQTYICNVNHKP | |
| | | SNTKVDKKVEPKSCDKTHT | |
| | Full light chain | SDIQMTQSPSSLSASVGDRVTITCRASQSVSS | 63 |
| | | AVAWYQQKPGKAPKLLIYSASSLYSGVPSRF | |
| | | SGSRSGTDFTLTISSLQPEDFATYYCQQSYGS | |
| | | LITFGQGTKVEIKRTVAAPSVFIFPPSDSQLKS | |
| | | GTASVVCLLNNFYPREAKVQWKVDNALQS | |
| | | GNSQESVTEQDSKDSTYSLSSTLTLSKADYE | |
| | | KHKVYACEVTHQGLSSPVTKSFNRGEC | |
| | Single chain | SDIQMTQSPSSLSASVGDRVTITCRASQSVSS | 64 |
| | variable fragment | AVAWYQQKPGKAPKLLIYSASSLYSGVPSRF | |
| | (VL-VH) | SGSRSGTDFTLTISSLQPEDFATYYCQQSYGS | |
| | | LITFGQGTKVEIKGTTAASGSSGGSSSGASSS | |
| | | EVQLVESGGGLVQPGGSLRLSCAASGFNVSS | |
| | | SYIHWVRQAPGKGLEWVAYIYPYSGYTSYA | |
| | | DSVKGRFTISADTSKNTAYLQMNSLRAEDTA | |
| | | VYYCARNYGGYPRKYAGAFDYWGQGTLVT | |
| | | VSSA | |
| R3H8 | HCDR1 | FSSSYI | 65 |
| | HCDR2 | SISSSSGSTS | 66 |
| | HCDR3 | FSYMSGSVFWAL | 67 |
| | LCDR1 | SVSSA | 68 |
| | LCDR2 | SASSLYS | 69 |
| | LCDR3 | SSWFPI | 70 |
| | VH | EISEVQLVESGGGLVQPGGSLRLSCAASGFN | 71 |
| | | FSSSYIHWVRQAPGKGLEWVASISSSSGSTSY | |
| | | ADSVKGRFTISADTSKNTAYLQMNSLRAEDT | |
| | | AVYYCARFSYMSGSVFWALDYWGQGTLVT | |
| | | V | |
| | VL | SDIQMTQSPSSLSASVGDRVTITCRASQSVSS | 72 |
| | | AVAWYQQKPGKAPKLLIYSASSLYSGVPSRF | |
| | | SGSRSGTDFTLTISSLQPEDFATYYCQQSSWF | |
| | | PITFGQGTKVEIKRT | |
| | Full heavy chain | EISEVQLVESGGGLVQPGGSLRLSCAASGFN | 73 |
| | | FSSSYIHWVRQAPGKGLEWVASISSSSGSTSY | |
| | | ADSVKGRFTISADTSKNTAYLQMNSLRAEDT | |
| | | AVYYCARFSYMSGSVFWALDYWGQGTLVT | |
| | | VSSASTKGPSVFPLAPSSKSTSGGTAALGCLV | |
| | | KDYFPEPVTVSWNSGALTSGVHTFPAVLQSS | |
| | | GLYSLSSVVTVPSSSLGTQTYICNVNHKPSNT | |
| | | KVDKKVEPKSCDKTHT | |
| | Full light chain | SDIQMTQSPSSLSASVGDRVTITCRASQSVSS | 74 |
| | | AVAWYQQKPGKAPKLLIYSASSLYSGVPSRF | |
| | | SGSRSGTDFTLTISSLQPEDFATYYCQQSSWF | |
| | | PITFGQGTKVEIKRTVAAPSVFIFPPSDSQLKS | |
| | | GTASVVCLLNNFYPREAKVQWKVDNALQS | |
| | | GNSQESVTEQDSKDSTYSLSSTLTLSKADYE | |
| | | KHKVYACEVTHQGLSSPVTKSFNRGEC | |
| | Single chain | SDIQMTQSPSSLSASVGDRVTITCRASQSVSS | 75 |
| | variable fragment | AVAWYQQKPGKAPKLLIYSASSLYSGVPSRF | |
| | (VL-VH) | SGSRSGTDFTLTISSLQPEDFATYYCQQSSWF | |
| | | PITFGQGTKVEIKGTTAASGSSGGSSSGASSS | |
| | | EVQLVESGGGLVQPGGSLRLSCAASGFNFSS | |
| | | SYIHWVRQAPGKGLEWVASISSSSGSTSYAD | |
| | | SVKGRFTISADTSKNTAYLQMNSLRAEDTAV | |
| | | YYCARFSYMSGSVFWALDYWGQGTLVTVS | |
| | | SA | |
| R3G9 | HCDR1 | LSSSSI | 76 |
| | HCDR2 | SISSYSGYTY | 77 |
| | HCDR3 | YSYKYWGWGEGM | 78 |
| | LCDR1 | SVSSA | 79 |
| | LCDR2 | SASSLYS | 80 |
| | LCDR3 | GYYDLI | 81 |
| | VH | EISEVQLVESGGGLVQPGGSLRLSCAASGFN | 82 |
| | | LSSSSIHWVRQAPGKGLEWVASISSYSGYTY | |
| | | YADSVKGRFTISADTSKNTAYLQMNSLRAE | |
| | | DTAVYYCARYSYKYWGWGEGMDYWGQGT | |
| | | LVTV | |
| | VL | SDIQMTQSPSSLSASVGDRVTITCRASQSVSS | 83 |
| | | AVAWYQQKPGKAPKLLIYSASSLYSGVPSRF | |
| | | SGSRSGTDFTLTISSLQPEDFATYYCQQGYY | |
| | | DLITFGQGTKVEIKRT | |
| | Full heavy chain | EISEVQLVESGGGLVQPGGSLRLSCAASGFN | 84 |
| | | LSSSSIHWVRQAPGKGLEWVASISSYSGYTY | |
| | | YADSVKGRFTISADTSKNTAYLQMNSLRAE | |
| | | DTAVYYCARYSYKYWGWGEGMDYWGQGT | |
| | | LVTVSSASTKGPSVFPLAPSSKSTSGGTAALG | |
| | | CLVKDYFPEPVTVSWNSGALTSGVHTFPAVL | |
| | | QSSGLYSLSSVVTVPSSSLGTQTYICNVNHKP | |
| | | SNTKVDKKVEPKSCDKTHT | |
| | Full light chain | SDIQMTQSPSSLSASVGDRVTITCRASQSVSS | 85 |
| | | AVAWYQQKPGKAPKLLIYSASSLYSGVPSRF | |
| | | SGSRSGTDFTLTISSLQPEDFATYYCQQGYY | |
| | | DLITFGQGTKVEIKRTVAAPSVFIFPPSDSQLK | |
| | | SGTASVVCLLNNFYPREAKVQWKVDNALQS | |
| | | GNSQESVTEQDSKDSTYSLSSTLTLSKADYE | |
| | | KHKVYACEVTHQGLSSPVTKSFNRGEC | |
| | Single chain | SDIQMTQSPSSLSASVGDRVTITCRASQSVSS | 86 |
| | variable fragment | AVAWYQQKPGKAPKLLIYSASSLYSGVPSRF | |
| | (VL-VH) | SGSRSGTDFTLTISSLQPEDFATYYCQQGYY | |
| | | DLITFGQGTKVEIKGTTAASGSSGGSSSGASS | |
| | | SEVQLVESGGGLVQPGGSLRLSCAASGENLS | |
| | | SSSIHWVRQAPGKGLEWVASISSYSGYTYYA | |
| | | DSVKGRFTISADTSKNTAYLQMNSLRAEDTA | |
| | | VYYCARYSYKYWGWGEGMDYWGQGTLVT | |
| | | VSSA | |

### 1. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### 2. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within +2 are included, in other embodiments, those which are within ±1 are included, and in still other embodiments, those within 0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### IV. NUCLEIC ACIDS

In certain embodiments, nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing the engineered cells of the disclosure. In a preferred embodiment, the nucleic acid molecules are oligonucleotides.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### V. POLYPEPTIDE EXPRESSION

In some aspects, there are nucleic acid molecule encoding polypeptides, antibodies, or antigen binding fragments of the disclosure. The nucleic acid molecules may be used to express large quantities of polypeptides. If the nucleic acid molecules are derived from a non-human, non-transgenic animal, the nucleic acid molecules may be used for humanization of the antibody or TCR genes.

### A. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody heavy and/or light chain, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### VI. THERAPY

In some aspects, the disclosed methods comprise administering an antibody or antigen binding fragment (including any of those described herein) to a patient. In some aspects, the patient is a cancer patient. In some aspects, the patient has a metabolic disease.

In some aspects, the disclosed methods comprise administering a cancer therapy, which can include any of the compositions described herein, to a subject or patient. The cancer therapy may be chosen based on an expression level measurement, alone or in combination with the clinical risk score calculated for the subject. The cancer therapy may be chosen based on a genotype of a subject. The cancer therapy may be chosen based on the presence or absence of one or more polymorphisms in a subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be a checkpoint inhibitor therapy. Any of these cancer therapies may also be excluded. Combinations of these therapies may also be administered.

The term “metabolic disease” as used herein, may include a diseases that disrupt metabolic processes in the patient. In certain asepcts, the metabolic disease includes disruption in an mTOR pathway. Metabolic diseases can include cancers, neurodegeneration, and diabetes. In some aspects, treating or preventing a metabolic disease includes maintaining metabolic homeostasis in the patient or in cells of the patient.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is a Stage I cancer. In some aspects, the cancer is a Stage II cancer. In some aspects, the cancer is a Stage III cancer. In some aspects, the cancer is a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some aspects, the cancer is aggressive cancer. In some aspects, the cancer is Stage I cancer. In some aspects, the cancer is Stage II cancer (e.g., IIA, IIB, IIC). In some aspects, the cancer is Stage III cancer (e.g., IIIA, IIIB, IIIC). In some aspects, the cancer is Stage IV cancer (e.g., IVA, IVB).

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### A. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

### 1. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### B. Checkpoint Inhibitors and Combination Treatment

Embodiments of the disclosure may include administration of immune checkpoint inhibitors, which are further described below.

### 1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### 2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### C. Combination Therapies

In some aspects, the methods may further comprise administration of an additional therapy such as radiotherapy, cancer immunotherapy, oncolytic virus, chemotherapies, hormone therapies, or surgery. Specific chemotherapies include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane), antimicrotubule agents, e.g., Paclitaxel (“Taxol”) and doxorubicin hydrochloride (“doxorubicin”), pyrimidine analogs, such as cytarabine (cytosine arabinoside), 5-fluorouracil (fluouracil; 5-FU) and floxuridine (fluorode-oxyuridine; FudR).

### VII. PHARMACEUTICAL COMPOSITIONS

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., compositions comprising TCR-binding polypeptides) are administered to a subject intravenously.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 M or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VIII. DETECTABLE LABELS

In some aspects of this disclosure, it will be useful to detectably or therapeutically label a Fab polypeptide or protein G Fab-binding domain. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

### IX. SAMPLE PREPARATION

In certain aspects, methods involve obtaining or evaluating a sample from a subject. The sample may include a sample obtained from any source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### X. HOST CELLS

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In certain embodiments transfection can be carried out on any prokaryotic or eukaryotic cell. In some aspects electroporation involves transfection of a human cell. In other aspects electroporation involves transfection of an animal cell. In certain aspects transfection involves transfection of a cell line or a hybrid cell type. In some aspects the cell or cells being transfected are cancer cells, tumor cells or immortalized cells. In some instances tumor, cancer, immortalized cells or cell lines are induced and in other instances tumor, cancer, immortalized cells or cell lines enter their respective state or condition naturally. In certain aspects the cells or cell lines can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NS0, Panc-1, PC12, PC-3, Peripheral blood cells, Plasma cells, Primary Fibroblasts, RBL, Renca, RLE, SF21, SF9, SH-SY5Y, SK-MES-1, SK-N-SH, SL3, SW403, Stimulus-triggered Acquisition of Pluripotency (STAP) cell or derivate SW403, T-cells, THP-1, Tumor cells, U2OS, U937, peripheral blood lymphocytes, expanded T cells, hematopoietic stem cells, or Vero cells.

### XI. KITS

Certain aspects of the present invention also concern kits containing compositions of the disclosure or compositions to implement methods of the disclosure. In some embodiments, kits can be used to detect the presence of a cancer cell in a sample. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, a kit contains one or more polypeptides capable of binding to a TCR (including a Vγ9Vδ2 TCR), including polypeptides disclosed herein. For example, a kit may comprise at least 1, 2, 3, 4, 5, 6, 7, 8, 9, 10 or more antibodies or antibody fragments (e.g., Fabs) disclosed herein for detecting a TCR (including a Vγ9Vδ2 TCR), in some cases for detecting TCR (including a Vγ9Vδ2 TCR) on expressed by a T cell. In some embodiments, a kit comprises a detection pair. In some embodiments, a kit comprises an enzyme. In some embodiments, a kit comprises a substrate for an enzyme.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. In certain aspects, negative and/or positive control nucleic acids, probes, and inhibitors are included in some kit embodiments.

Kits may further comprise instructions for use. For example, in some embodiments, a kit comprises instructions for detecting a TCR (including a Vγ9Vδ2 TCR) in a sample.

It is contemplated that any method or composition described herein can be implemented with respect to any other method or composition described herein and that different embodiments may be combined. The claims originally filed are contemplated to cover claims that are multiply dependent on any filed claim or combination of filed claims.

### XII. EXAMPLES

The following examples are included to demonstrate certain embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—Conformation-Specific Synthetic Intrabodies Modulate mTOR Signaling with Subcellular Spatial Resolution

Engineered sABs Selectively Obstruct the mTOR^{FRB} Substrate Recruitment Site.

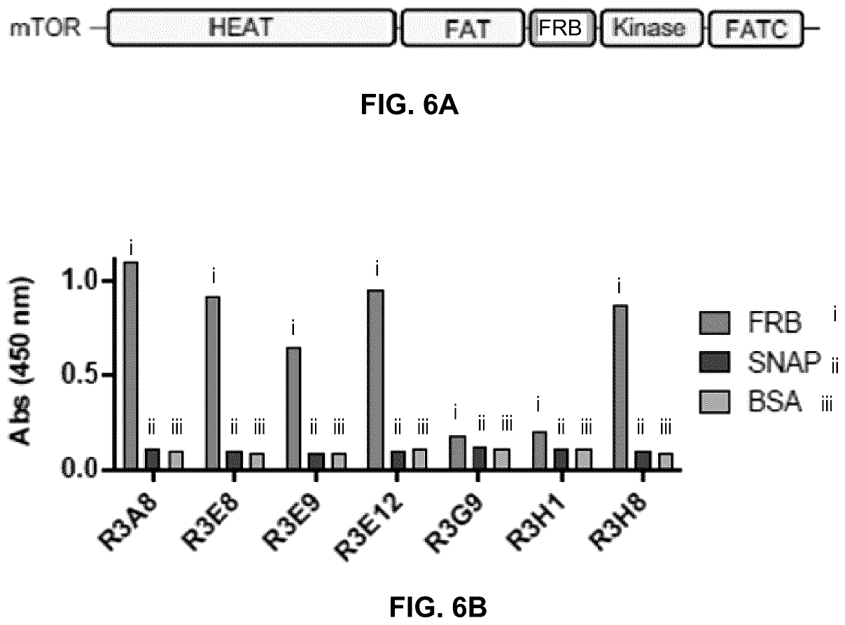

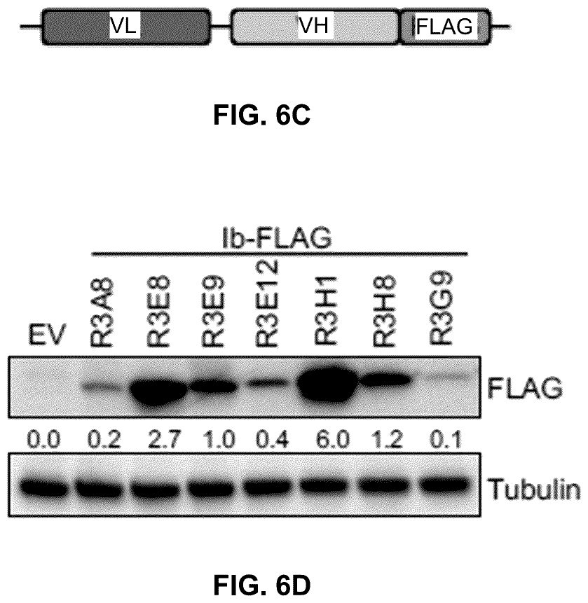

The mTOR^{FRB} domain is a small (11 kDa) 4-helix bundle that facilitates the entry of some mTORC1 substrates into the catalytic cleft (41,42). Synthetic antibodies (sABs) were designed to engage with and modulate specific epitopes within the mTOR signaling pathway by exploiting this stable and functional domain. Recombinant SNAP-tagged human mTOR^{FRB} was purified, biotinylated, and used as a target for five rounds of phage display biopanning. The concentration of mTOR^{FRB} was incrementally reduced from 1 μM in the first round to 2 nM in the last round to apply stringent selection pressure while maintaining 1 μM non-biotinylated SNAP in solution. Phage ELISA identified seven unique phage clones exhibiting selective binding against mTOR^{FRB} for further characterization (FIGS. 6A and 6B). Based on a previously reported design (52), these clones were converted into scFv or “intrabody” format and tested for intracellular solubility in human Expi293F cells. All seven unique scFv constructs displayed detectable intracellular expression, albeit at varying levels (FIGS. 6C and 6D).

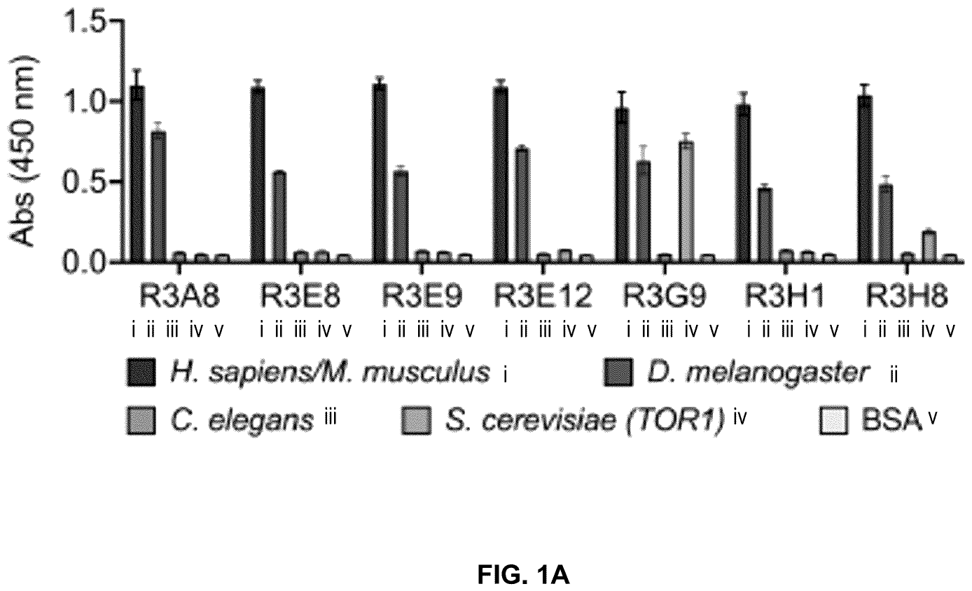

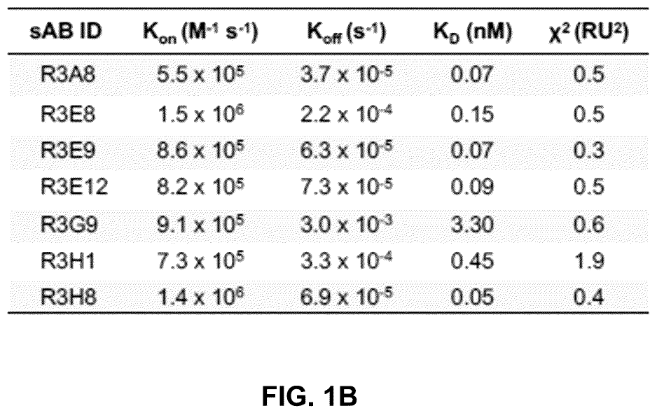

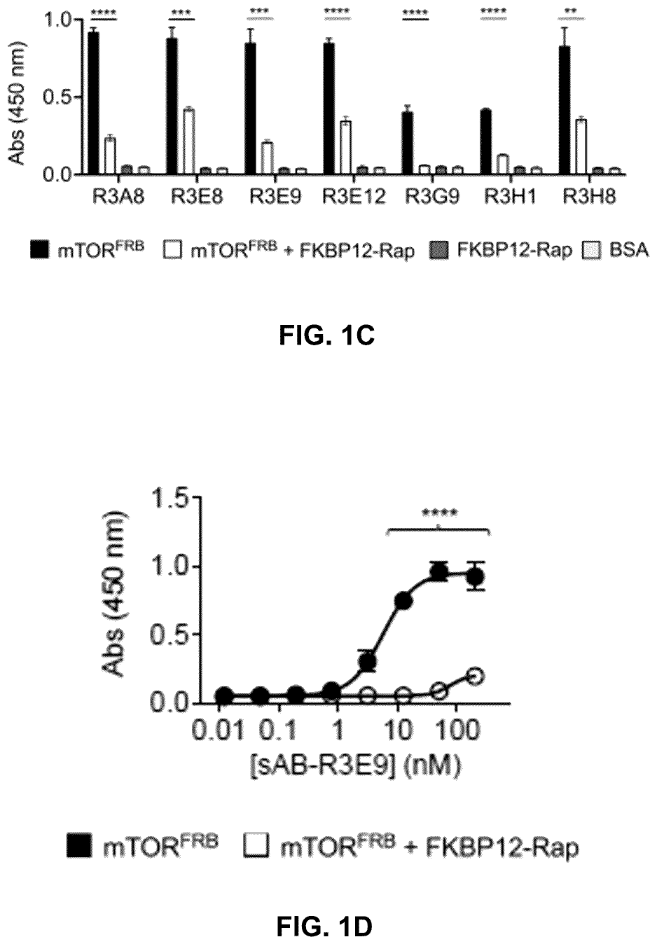

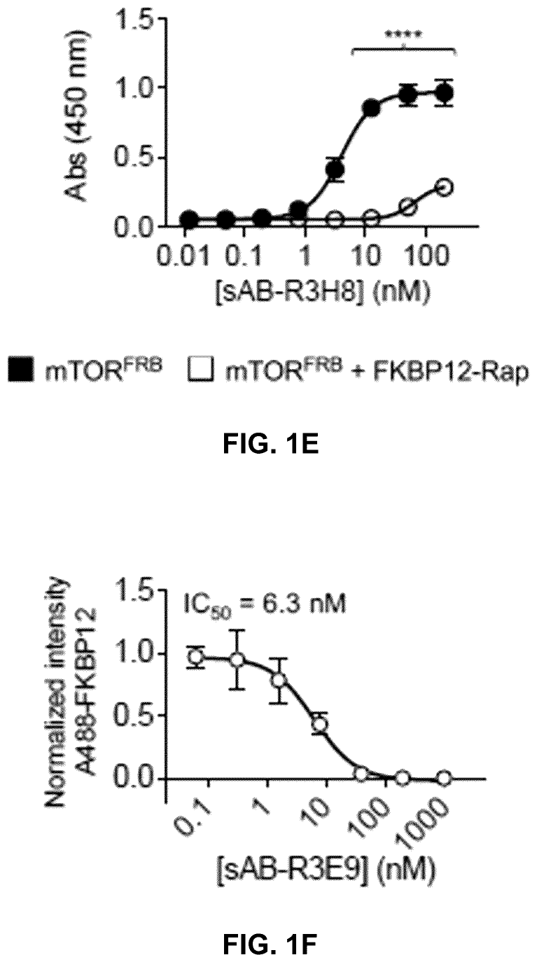

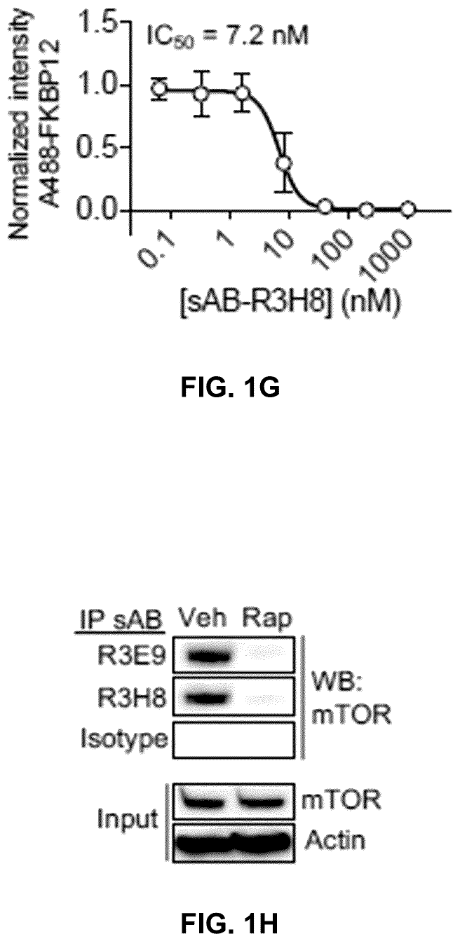

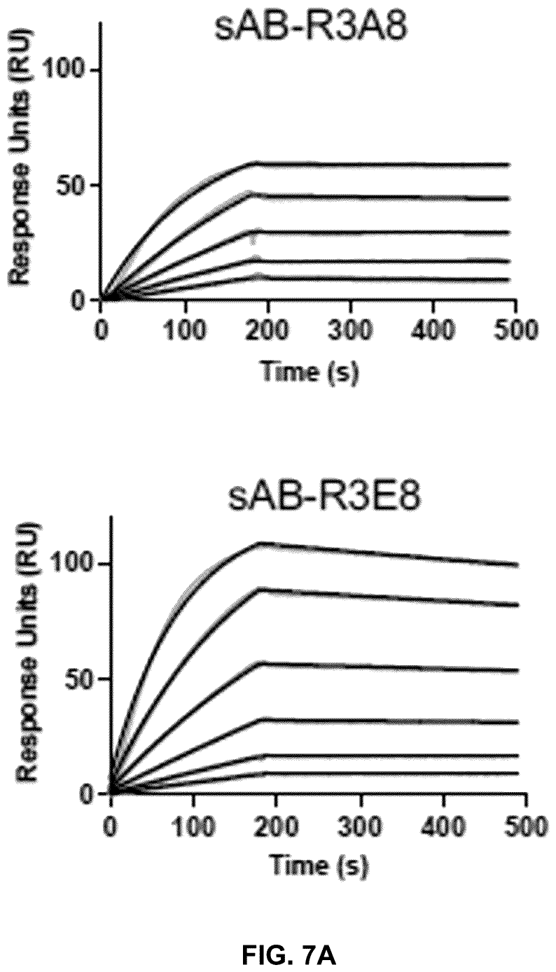

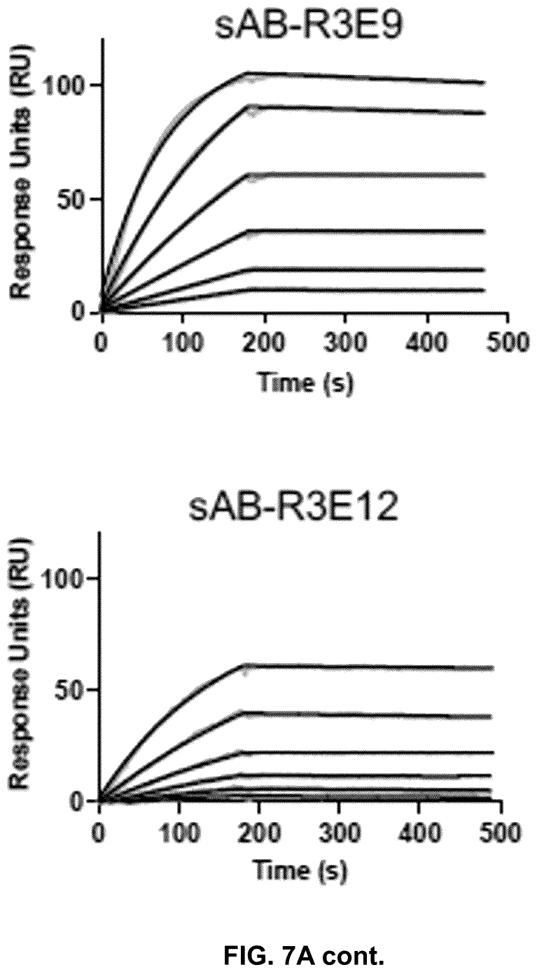

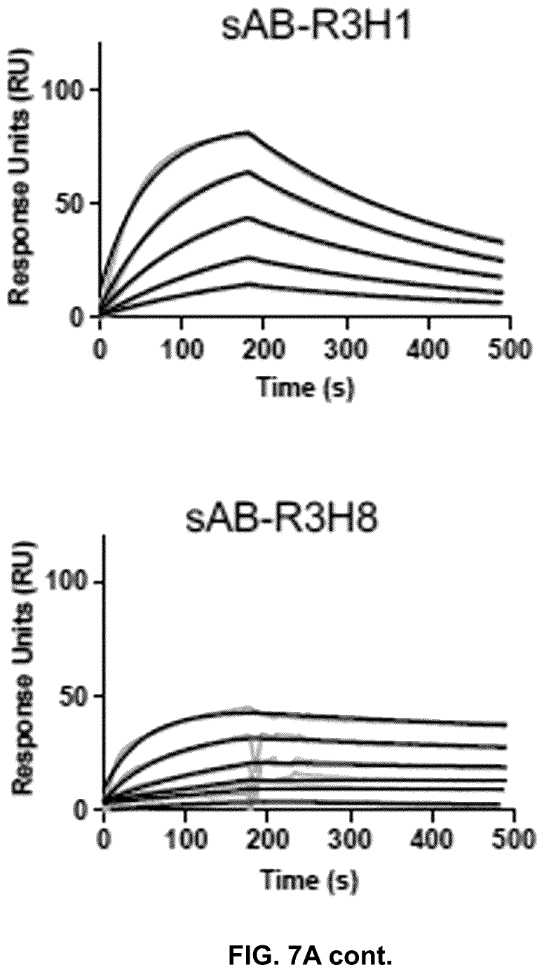

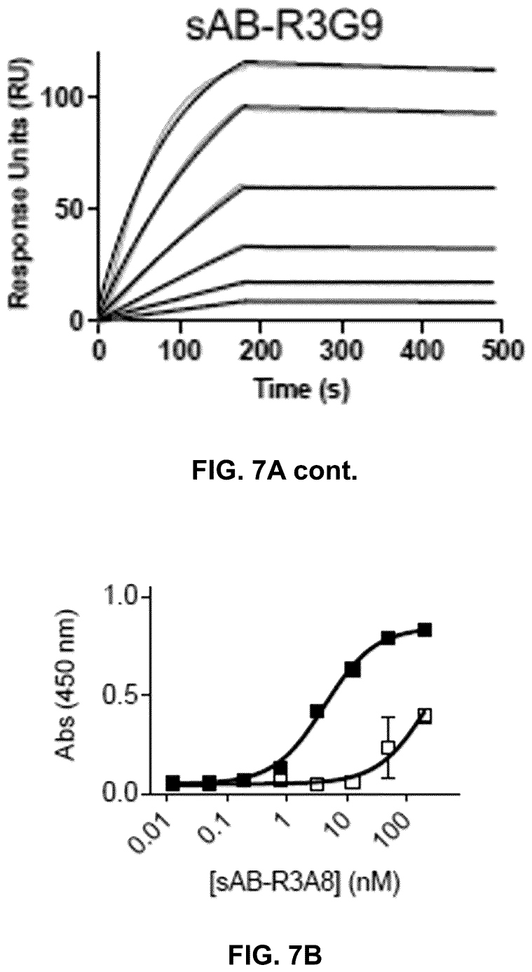

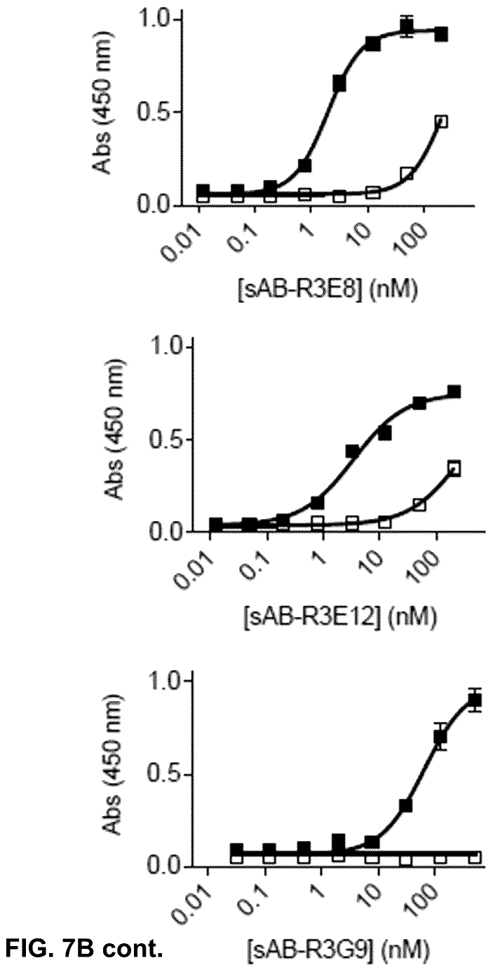

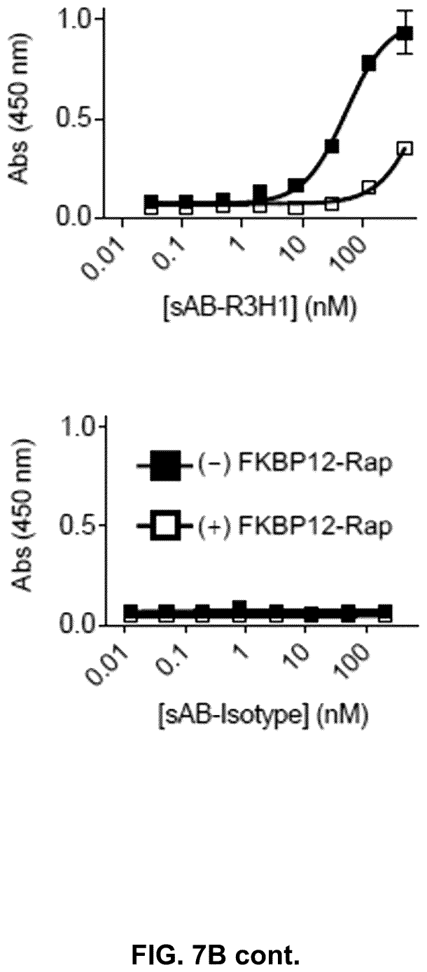

To evaluate their utility as affinity reagents in Fab format, the cross-reactivity of these sABs with mTOR^{FRB}/TOR^{FRB} domains from diverse model organisms was investigated. Due to the evolutionary conservation of mTOR in eukaryotes, all sABs displayed binding to mTOR^{FRB}/TOR^{FRB} from H. sapiens, M. musculus, and D. melanogaster (FIG. 1A). Additionally, sAB-R3G9 and sAB-R3H8 exhibited detectable binding to TOR1^{FRB} from S. cerevisiae, despite sharing only 54% sequence identity with human mTOR^{FRB}. Binding affinities were determined by surface plasmon resonance for these sABs against human mTOR^{FRB}, with K_{D} values ranging from 50 pM for the tightest binder, sAB-R3H8, to 3.3 nM for the weakest binder, sAB-R3G9 (FIGS. 1B and 7A).

Central to the goal of developing genetically encodable mTOR inhibitors was to determine if the epitopes engaged by these sABs overlap with the mTOR^{FRB} substrate recruitment site. Since endogenous mTORC1 substrates bind weakly to mTOR^{FRB}, FKBP12-Rapamycin was used as a surrogate competitor due to its favorable interaction with mTOR^{FRB} Competitive single-point and multi-point ELISA assessed sAB binding to mTOR^{FRB} in the presence or absence of 10 μM FKBP12-Rapamycin. This competitive screening indicated that all tested sABs engage the mTOR^{FRB} substrate recruitment site, leading to the selection of sAB-R3E9 and sAB-R3H8 as primary candidates for further development based on affinity considerations (FIGS. 1C-1E and 7B).

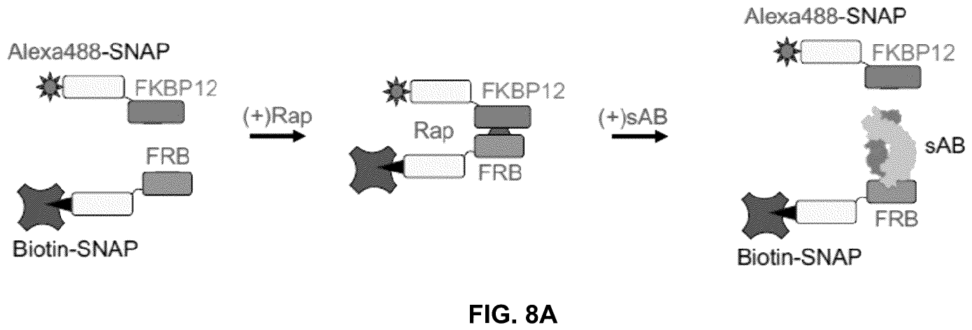

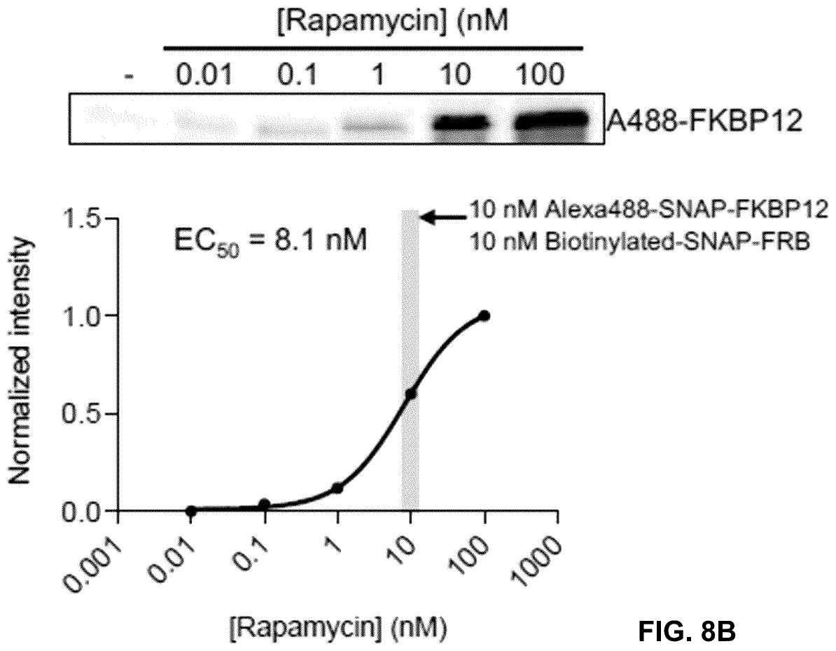

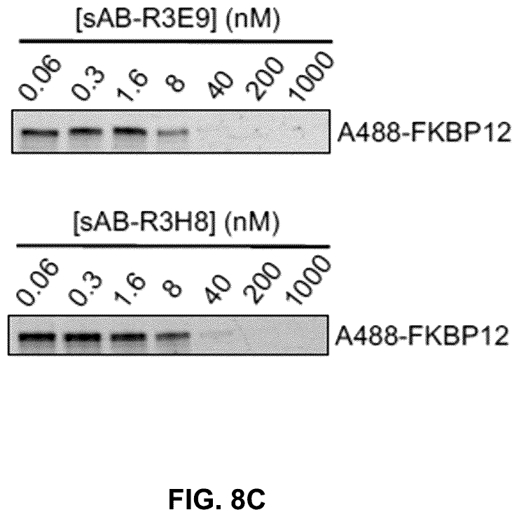

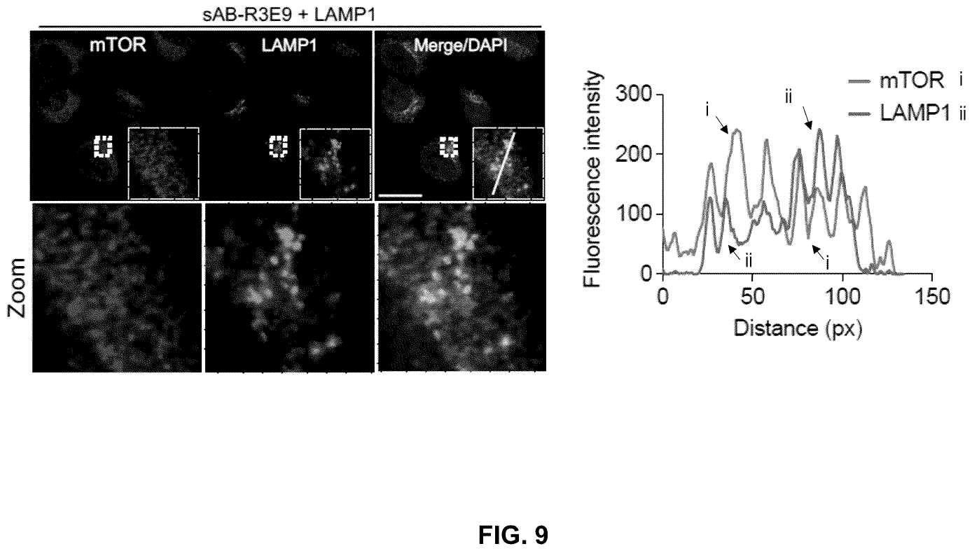

Residual sAB binding signals in the presence of a competitor prompted an investigation into whether the sAB and FKBP12-Rapamycin epitopes are partially or fully overlapped. An in vitro fluorescence-based pulldown assay was developed based on rapamycin-induced ternary complex formation of FKBP12 and mTOR^{FRB} to validate obstruction of the mTOR^{FRB} substrate recruitment site (FIGS. 8A and 8B). By titrating sAB-R3E9 or sAB-R3H8 into a mixture containing 10 nM biotinylated mTOR^{FRB}, 10 nM Alexa488-labeled SNAP-FKBP12, and 10 nM rapamycin, complete dose-dependent depletion of Alexa488-SNAP-FKBP12 was observed in streptavidin bead enrichment of biotinylated mTOR^{FRB} (FIG. 8C). The IC₅₀ values for sAB-R3E9 and sAB-R3H8 were determined to be 6.3 nM and 7.2 nM, respectively, for inhibiting the FKBP12-Rapamycin interaction with mTOR^{FRB} (FIGS. 1F and 1G). Furthermore, rapamycin-sensitive mTOR engagement and coordination of mTORC1 were confirmed by immunoprecipitation from human Expi293F cells treated with rapamycin or a vehicle control (FIGS. 1H and 1I). Lastly, HeLa cells were immunostained with sAB-R3E9 and an anti-LAMP1 antibody, visually confirming lysosomal mTORC1 localization alongside other cellular locations, including the nucleus (FIG. 9).

Crystal Structures of sAB-R3E9 and sAB-R3H8 Bound to mTOR^{FRB} Reveal mTORC1 Substrate Mimicry.

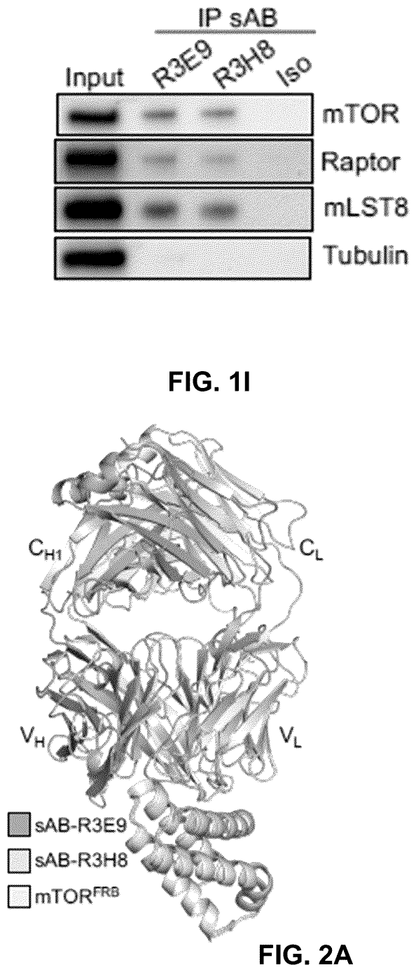

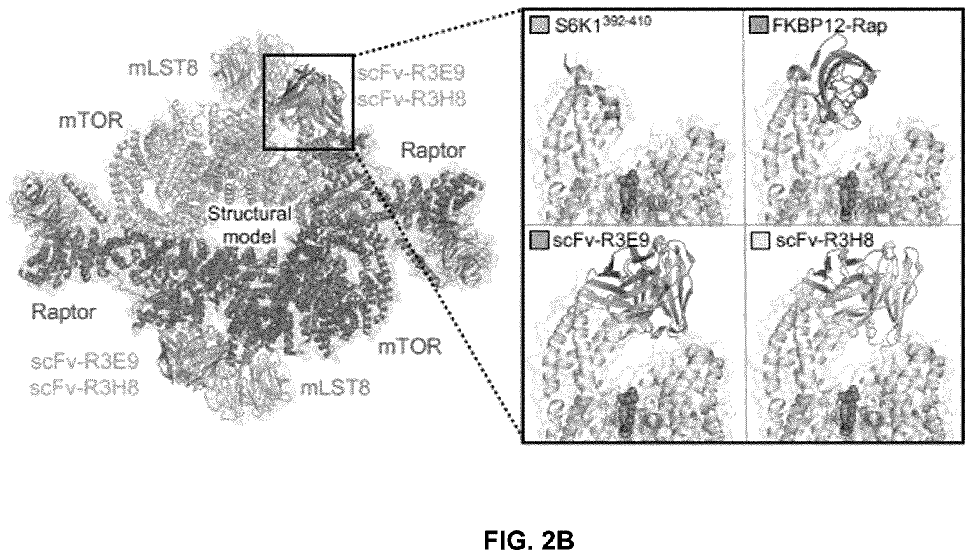

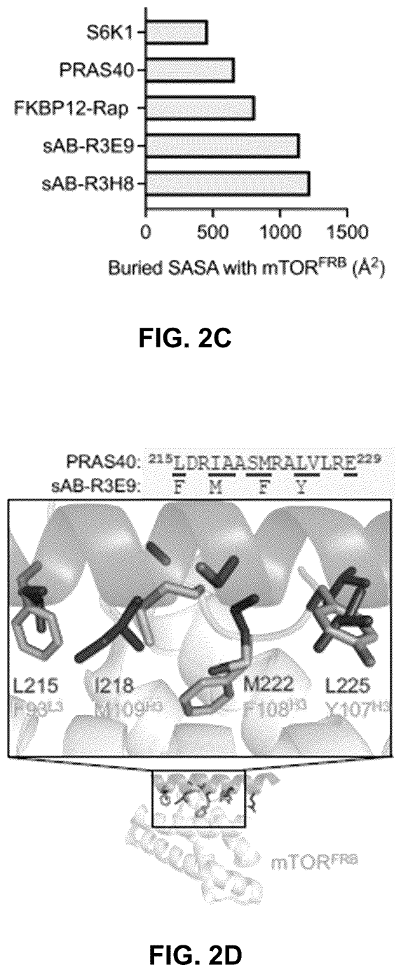

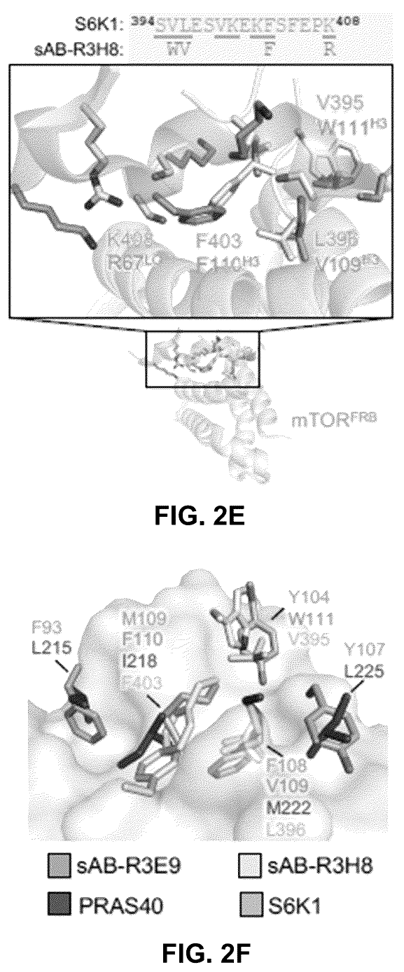

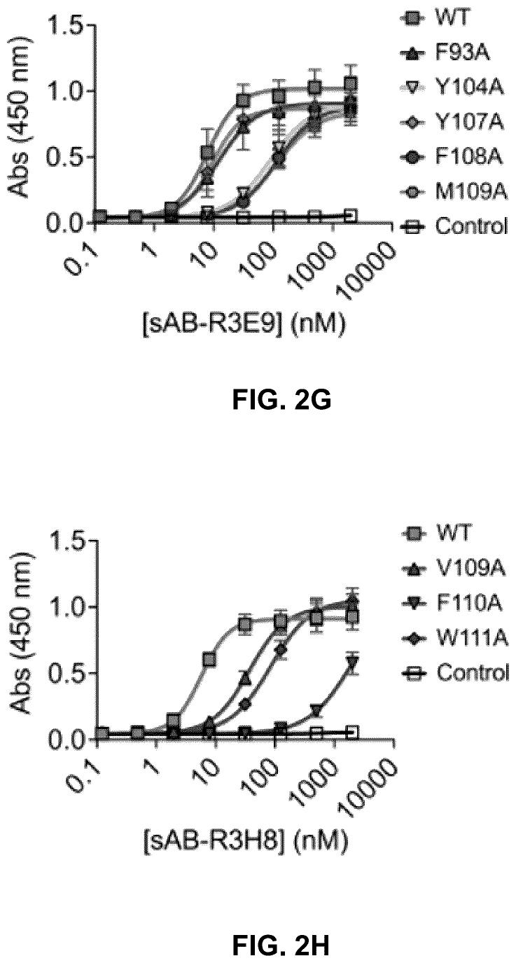

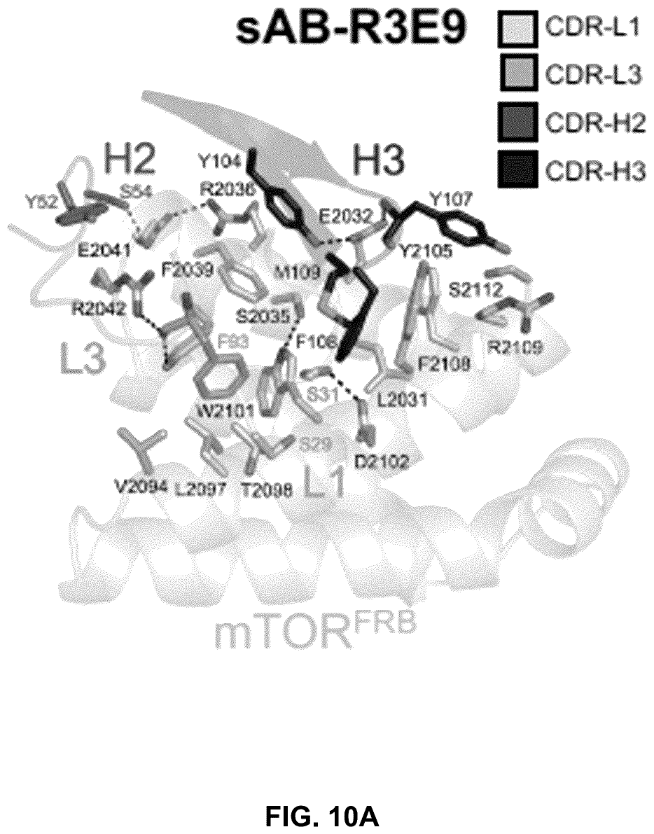

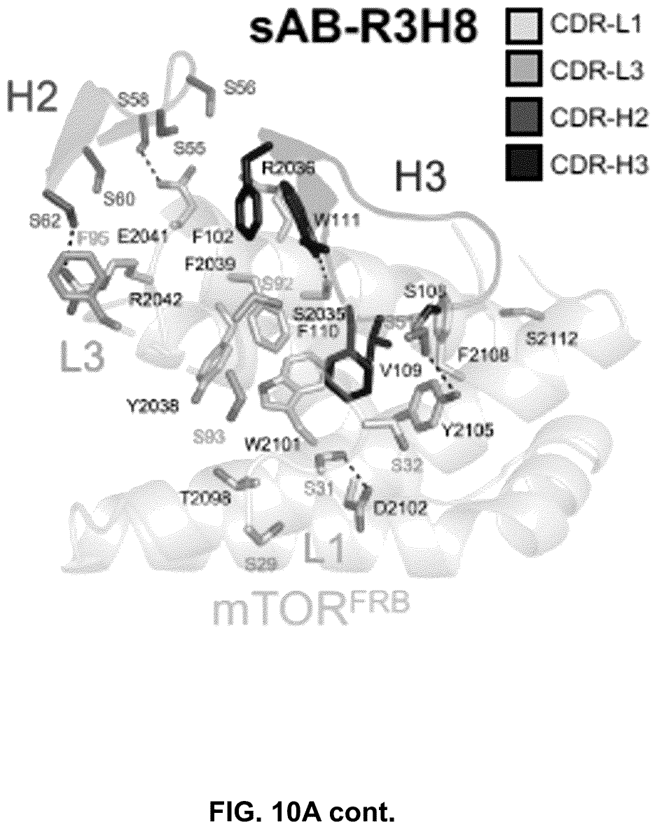

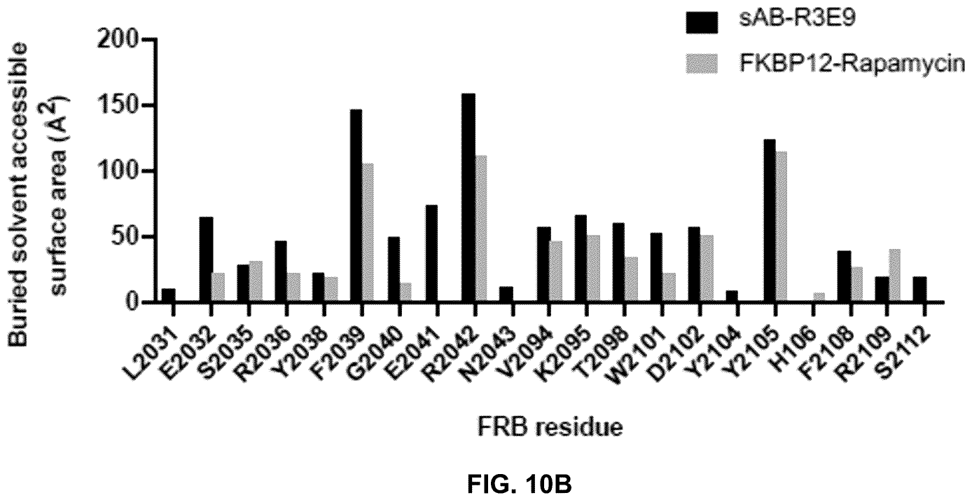

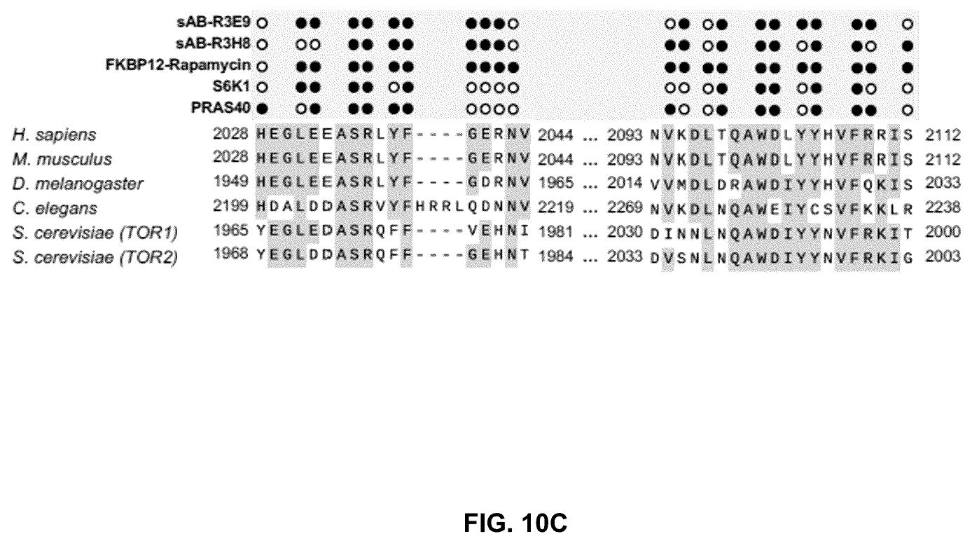

To elucidate the structural features underlying sAB recognition of the mTOR^{FRB} substrate recruitment site, independent crystal structures of sAB-R3E9 and sAB-R3H8 bound to mTOR^{FRB} were solved at 1.6 Å and 2.0 Å resolution, respectively (Table Si). The coordination of mTOR^{FRB} by sAB-R3E9 and sAB-R3H8 appeared highly similar, with differences primarily in the “elbow” angle connecting the CH1-CL and VH-VL domains (FIG. 2A). Extensive hydrogen bonding and Van der waals interactions along the relatively flat and hydrophobic substrate recruitment site provided the structural basis for the high binding affinities of 70 pM and 50 pM for sAB-R3E9 and sAB-R3H8, respectively (FIG. 10A). In agreement with binding assays, the paratopes of sAB-R3E9 and sAB-R3H8 are positioned to directly cover the FKBP12-Rapamycin binding interface, interacting with nearly every mTOR^{FRB} residue that FKBP12-Rapamycin engages (FIG. 10B). Alignment of the crystal structures of sAB-R3E9-mTOR^{FRB} and sAB-R3H8-mTOR^{FRB} with a previously determined cryo-EM structure of mTORC1 (PDB: 6BCX) confirmed that scFv-format binding is accessible to mTOR^{FRB}, with similar binding poses as S6K1³⁹²⁻⁴¹⁰ (PDB: 5WBH) and FKBP12-Rapamycin (PDB: 1FAP), illustrating their potential to obstruct substrate entry into the catalytic cleft (FIG. 2B).

Given these observations, an investigation was conducted to determine whether sAB-R3E9 and sAB-R3H8 share molecular recognition features with structurally characterized mTOR^{FRB}-dependent mTORC1 substrates. Multiple sequence alignment of mTOR^{FRB} showed that many residues contacted by these sABs are evolutionarily conserved from yeast to humans (FIG. 10C). A quantitative analysis revealed solvent accessible surface area (SASA) burial of 1,148 Å² and 1,228 Å² by sAB-R3E9 and sAB-R3H8 with mTOR^{FRB} respectively (FIG. 2C). These SASA values are significantly greater than those for S6K1³⁹²⁻⁴¹⁰ PRAS40²¹²⁻²³², or FKBP12-Rapamycin, representing a favorable attribute toward obstruction of the mTOR active site. Examination of structural interactions revealed remarkable alignment between residues L215, I218, M222, and L225 from PRAS40²¹²⁻²³² and residues F93^{L3} Y107^{H3}, F108^{H3}, and M109H³ from sAB-R3E9 (FIG. 2D). Similarly, coordination between residues V395, L396, F403, and K408 from S6K1³⁹²⁻⁴¹⁰ and residues R67LC, V109^{H3}, F110^{H3}, and W111^{H3} from sAB-R3H8 was observed (FIG. 2E).

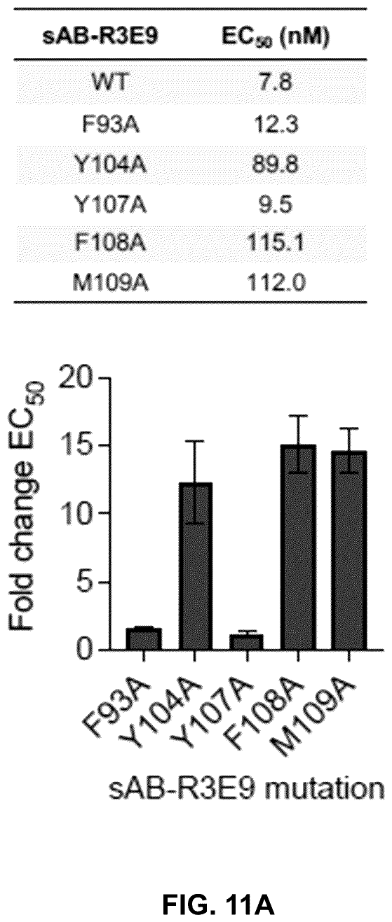

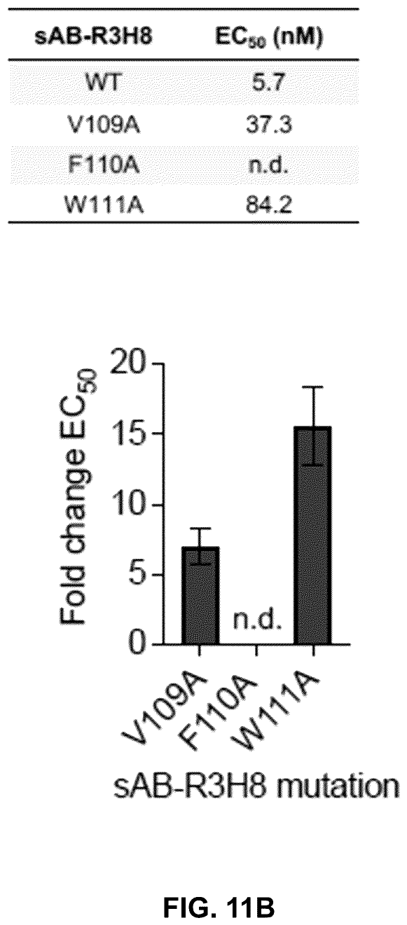

Overall, five highly conserved positions of hydrophobic residue coordination at the mTOR^{FRB} substrate recruitment site were identified among naturally occurring or synthetic molecules despite entirely different sequence and secondary structure characteristics (FIG. 2F). Alanine scanning mutagenesis revealed significant energetic contributions (EC₅₀ fold change >5-fold) toward mTOR^{FRB} binding by residues Y104^{H3}, F108^{H3}, and M109^{H3} in sAB-R3E9 and by residues V109^{H3}, F110^{H3}, and W111^{H3} in sAB-R3H8 (FIGS. 1G-1H and 11A-11B). These residues form a confined network at the center of the mTOR^{FRB} substrate recruitment site with almost no positional sequence identity between molecules. Together, crystal structure determination of two unique sABs bound to mTOR^{FRB} paired with structure-guided mutational binding assays provided molecular insight into the regulation of mTOR^{FRB}-dependent substrate recruitment. The findings suggest that mTOR^{FRB}-mediated coordination of mTORC1 substrates may be based primarily on a code of shape complementarity with low stringency for hydrophobic side chain stereochemistry at each position.

Conformation-Specific Intrabodies Highlight mTOR^{FRB} as an Allosteric Toggle Switch to Modulate the mTORC1 Architecture.

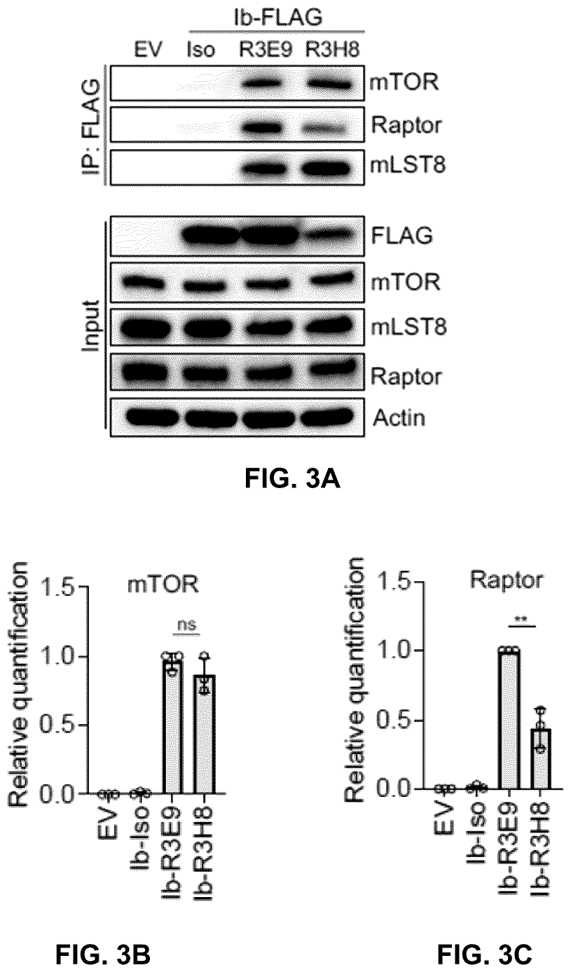

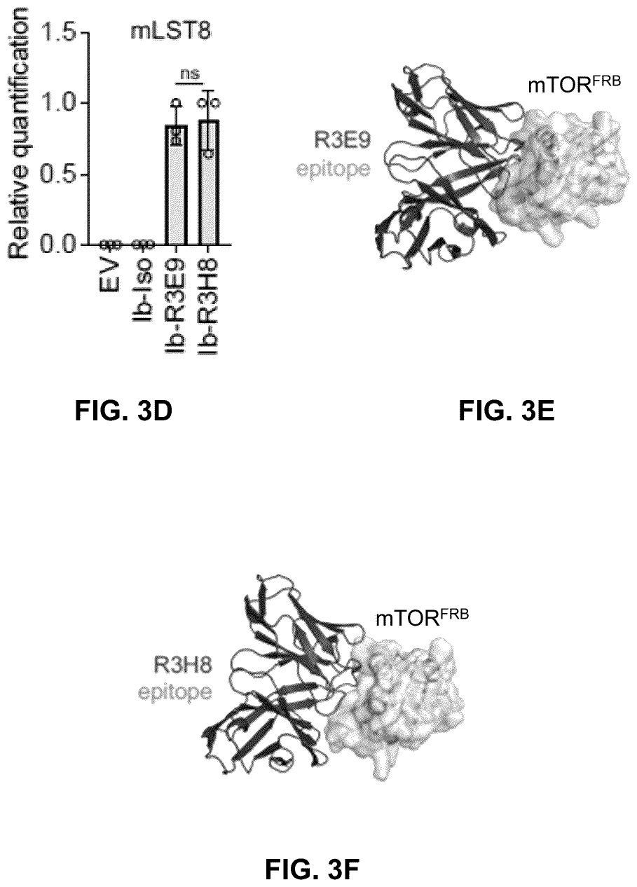

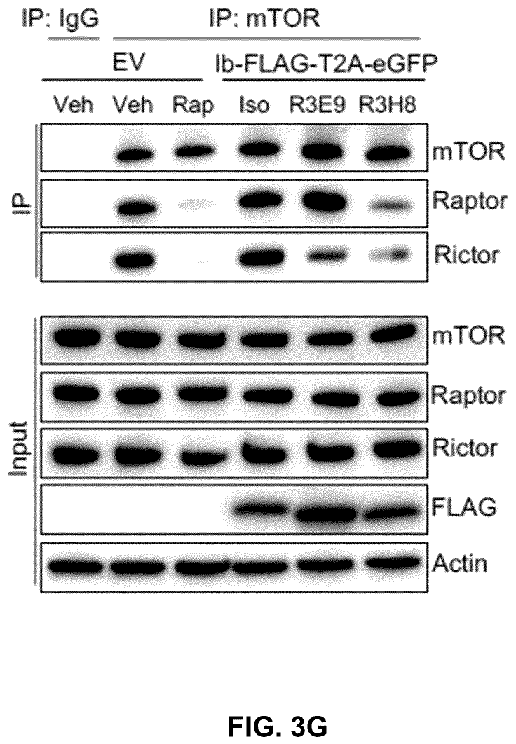

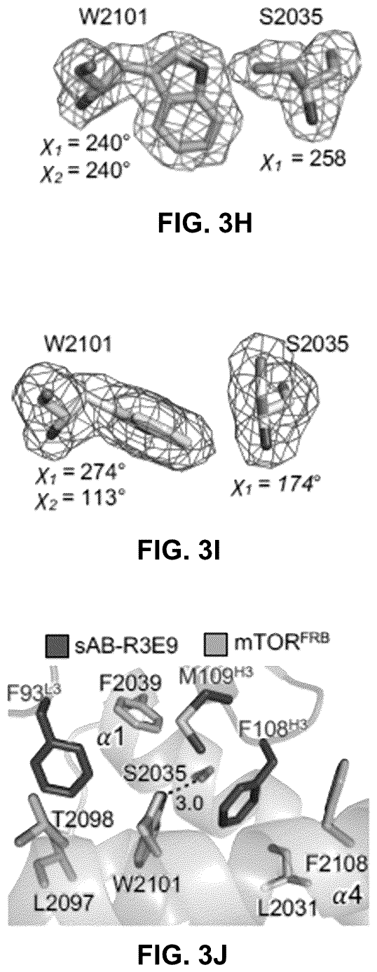

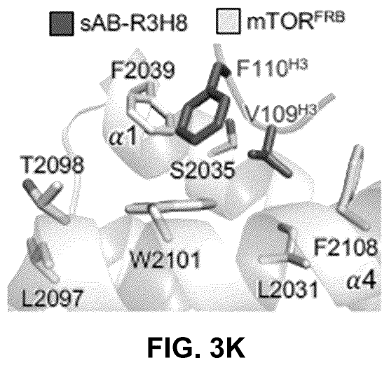

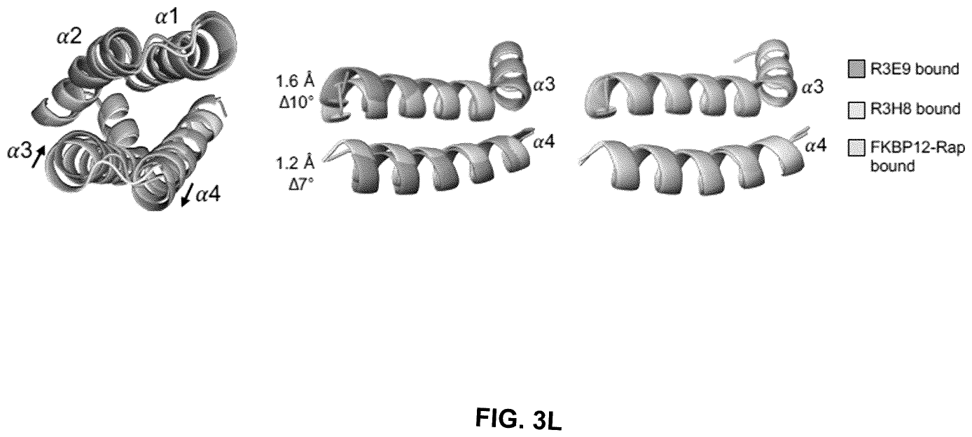

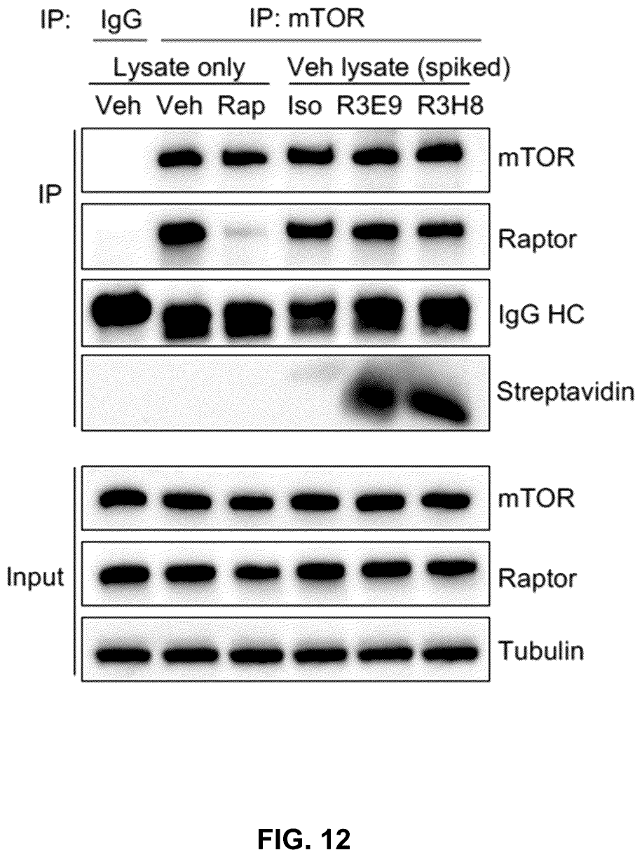

Motivated by observations of molecular mimicry between sABs and mTORC1 substrates, the potential for sAB-R3E9 and sAB-R3H8 to engage with mTORC1 within human cells as intrabodies was explored. These intrabodies, termed Ib-R3E9 and Ib-R3H8, were confirmed to engage intracellularly with mTORC1 through transfection experiments in Expi293F cells. Cells were transfected with a FLAG-tagged isotype control intrabody (Ib-Iso), Ib-R3E9, or Ib-R3H8 for 48 hours, followed by anti-FLAG immunoprecipitation. Both Ib-R3E9 and Ib-R3H8 successfully pulled down mTOR and core mTORC1 components, such as mLST8 and Raptor, indicating that intrabody target engagement was maintained in the reducing intracellular environment (FIG. 3A). Interestingly, a significant decrease in Raptor signal was observed in the Ib-R3H8 pulldown compared to Ib-R3E9, while signals for mTOR and mLST8 remained consistent between these intrabodies (FIGS. 3B-3D). This suggests that intracellular engagement of mTORC1 by Ib-R3E9 and Ib-R3H8 differentially influences the stability of the mTOR-Raptor interaction despite targeting nearly identical mTOR^{FRB} epitopes (FIGS. 3E-3F). This effect could not be replicated by pan-mTOR immunoprecipitation from cell lysates spiked with biotinylated sAB-R3E9 or sAB-R3H8, indicating that modulation of the mTOR-Raptor interaction by Ib-R3E9 or Ib-R3H8 is dependent on a native, active intracellular environment (FIG. 12).

The effects of intrabodies were compared to rapamycin to further understand how Ib-R3E9 and Ib-R3H8 modulate endogenous mTOR assemblies in cells. A pan-mTOR co-immunoprecipitation was performed in vehicle-treated cells, rapamycin-treated cells, and cells expressing FLAG-T2A-eGFP-tagged Ib-Iso, Ib-R3E9, or Ib-R3H8 isolated by fluorescence-activated cell sorting (FACS). Consistent with previous reports, rapamycin treatment for 24 hours markedly diminished interactions of both Raptor and Rictor with mTOR (FIG. 3G). The mTOR-Rictor interaction was obstructed similarly by both intrabodies, confirming that masking the mTOR^{FRB} substrate recruitment site is sufficient to obstruct the assembly of Rictor onto newly synthesized mTORC2 complexes. However, distinctive stabilizing or destabilizing effects on the mTOR-Raptor interaction were observed between cells expressing Ib-R3E9 or Ib-R3H8, respectively.

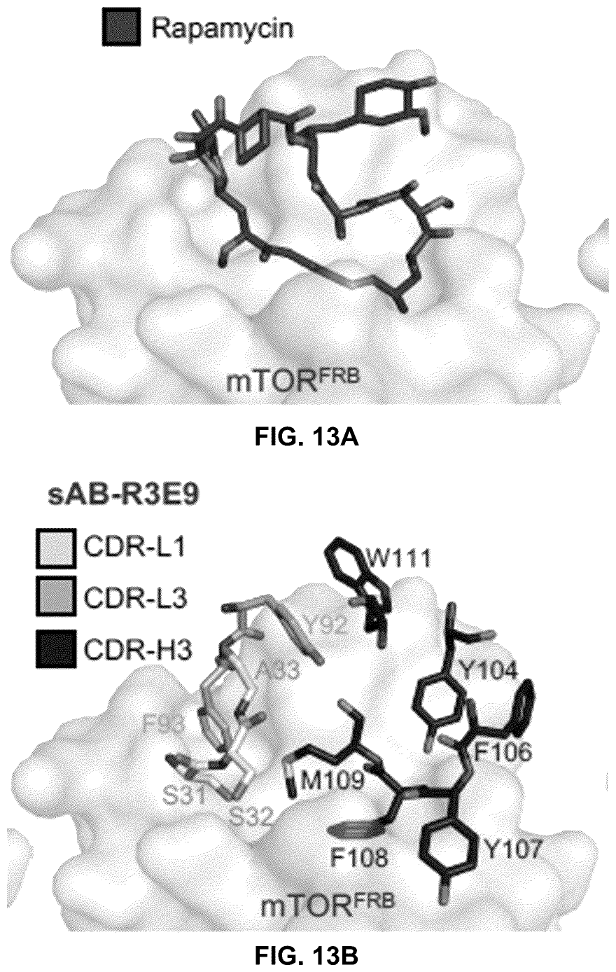

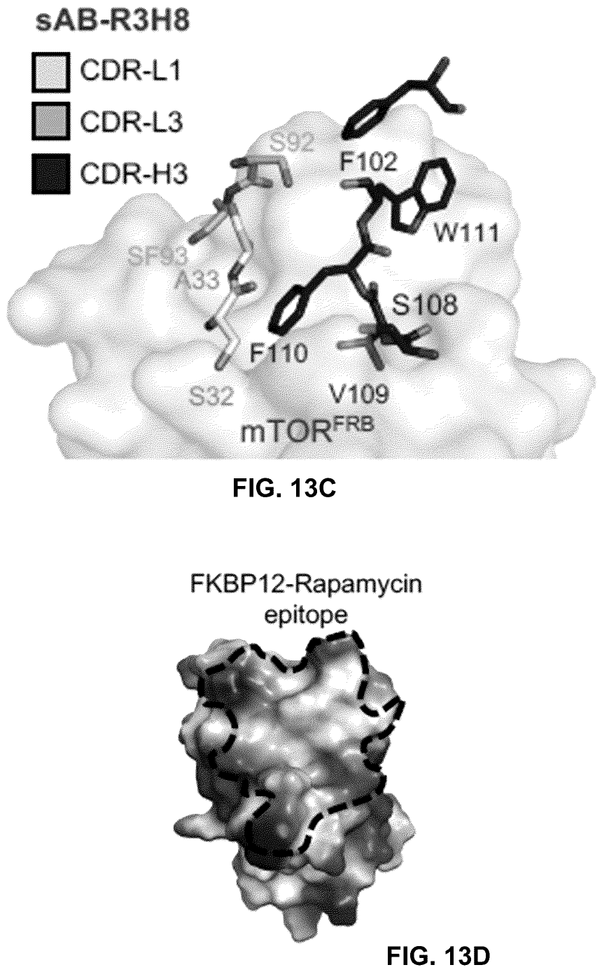

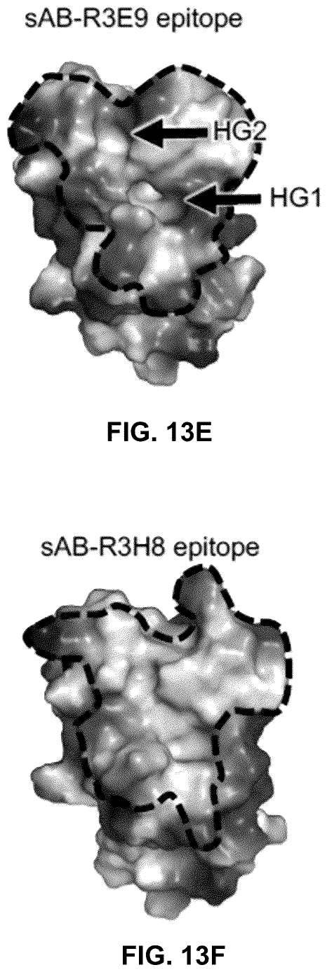

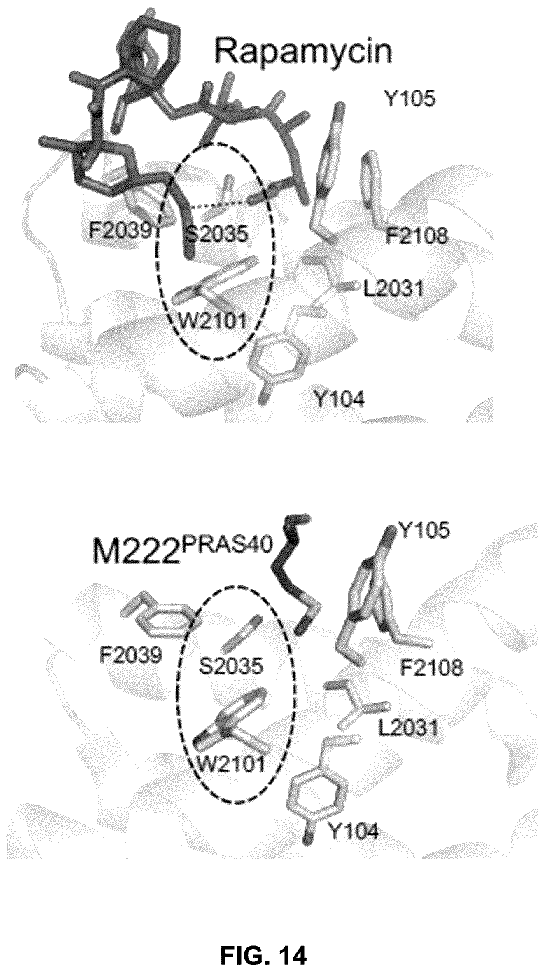

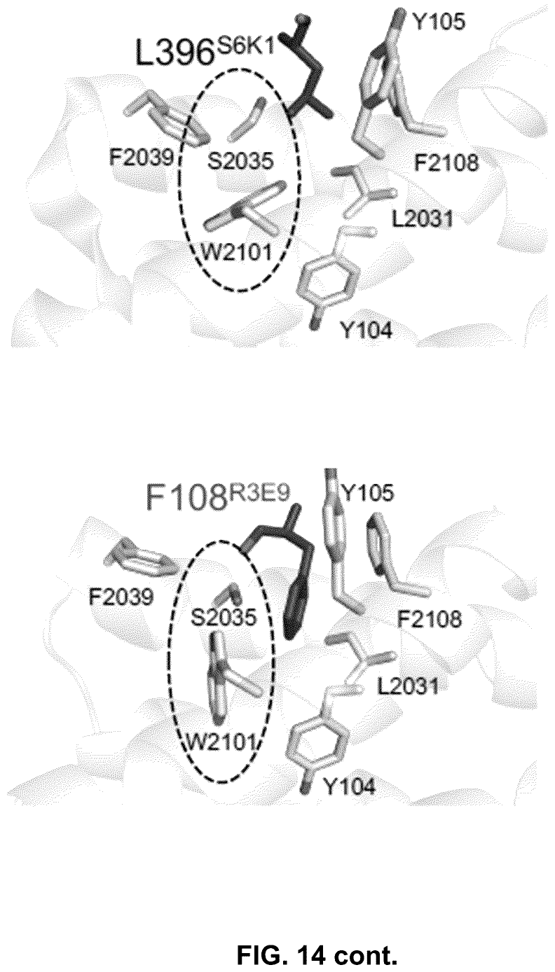

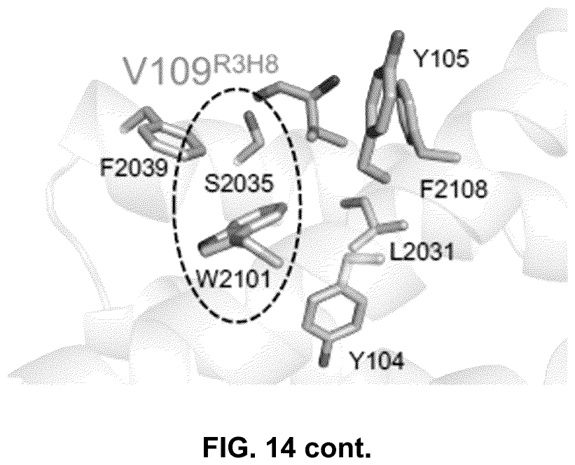

The allosteric destabilization of the mTOR-Raptor interaction by Ib-R3H8 but not by Ib-R3E9 suggests that engagement of the FKBP12-rapamycin binding site alone is insufficient to induce Raptor dissociation. The structural mechanism underlying intrabody-based modulation of the mTOR-Raptor interaction was elucidated by examining interactions at the mTOR^{FRB} interface. Despite their lack of chemical identity, rapamycin, sAB-R3E9, and sAB-R3H8 exhibit similar structural arrangement (FIGS. 13A-13C). Well-defined electron density shows a hydrogen bonding interaction between NE1 of W2101 and the hydroxyl of S2035 in mTOR^{FRB} bound by sAB-R3E9. Notably, the hydrogen bonding geometry requires both these sidechains to adopt unfavorable rotamer dihedral angles (FIGS. 3H-3I). The stabilization of this high energy configuration is probably influenced not only by the hydrogen bonding interaction but also by a nest of five aromatic residues that form a T-shaped and parallel π-stacking network between sAB-R3E9 (F93^{L3} and F108^{H3}) and mTOR^{FRB} (F2039, W2101, and F2108) (FIG. 3J). This configuration results in altered morphology of the HG1 cavity, providing the basis for the ~4 Å depth extension observed in the sAB-R3E9 bound state (FIGS. 13D-13F). In other mTOR^{FRB} structures bound by sAB-R3H8, S6K1³⁹²⁻⁴¹⁰, PRAS40²¹²⁻²³², or FKBP12-Rapamycin, W2101 and S2035 do not interact and are found in favorable, low-energy conformations (FIGS. 3K and 14).

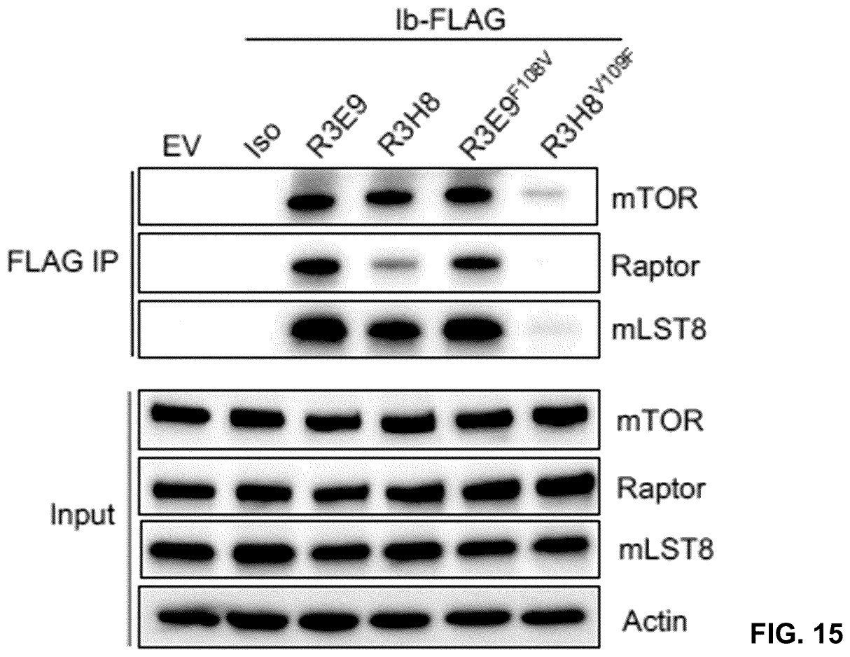

To determine whether sAB side chain stereochemistry alone influences the coordination of mTOR^{FRB}, an anti-FLAG immunoprecipitation was performed using variants sAB-R3E9^{F108V} and sAB-R3H8^{V109F}. The results suggested that swapping side chain properties in the shallow HG1 cavity is insufficient to influence the mTOR-Raptor interaction (FIG. 15). Consequently, intrabody-based allosteric tunability of the mTOR-Raptor interaction appears to rely on a combination of high-energy side chain rotamer stabilization along with subtle conformational rearrangements in mTOR^{FRB} helices α3 (1.6 Å, ~10°) and α4 (1.2 Å, ~7°) (FIG. 3L). The findings support a model where minor distortions in mTOR^{FRB} conformation can allosterically stabilize or destabilize the interaction between mTOR and Raptor. Future studies should explore whether distinct conformational states of mTOR^{FRB} have functional implications for regulating nutrient-dependent mTOR activity, complex assembly, or substrate coordination.

Ib-R3E9 and Ib-R3H8 Potently Inhibit mTOR Signaling in Human Cells.

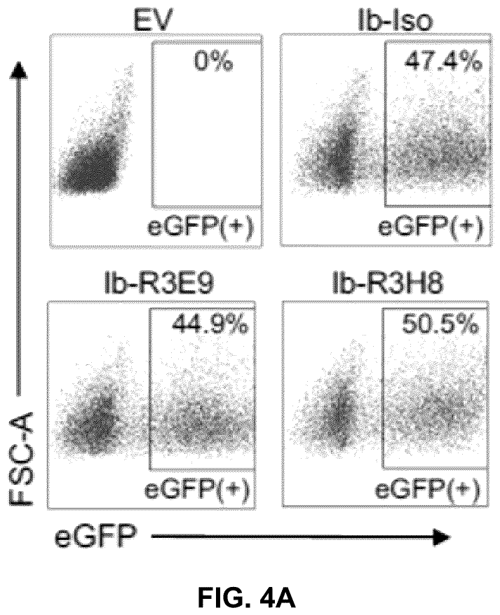

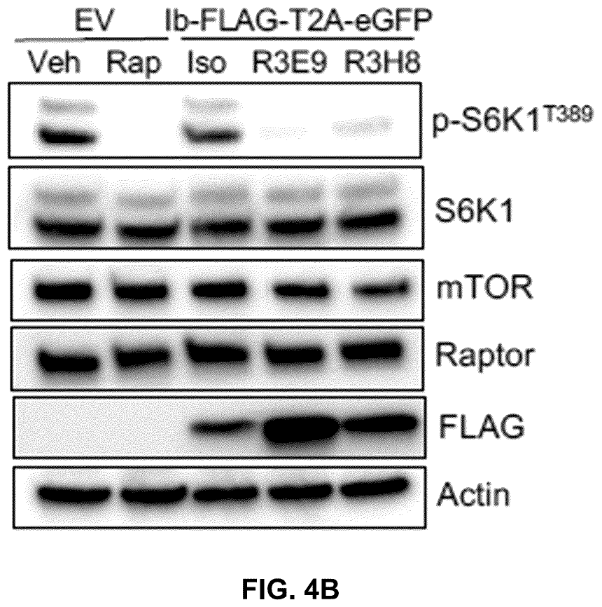

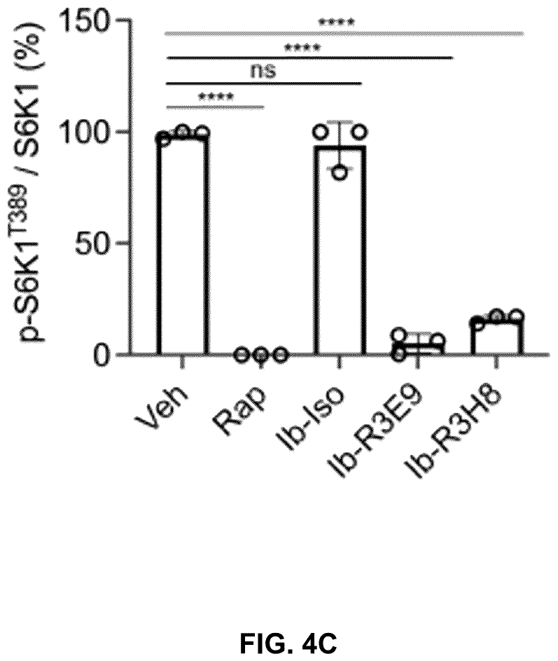

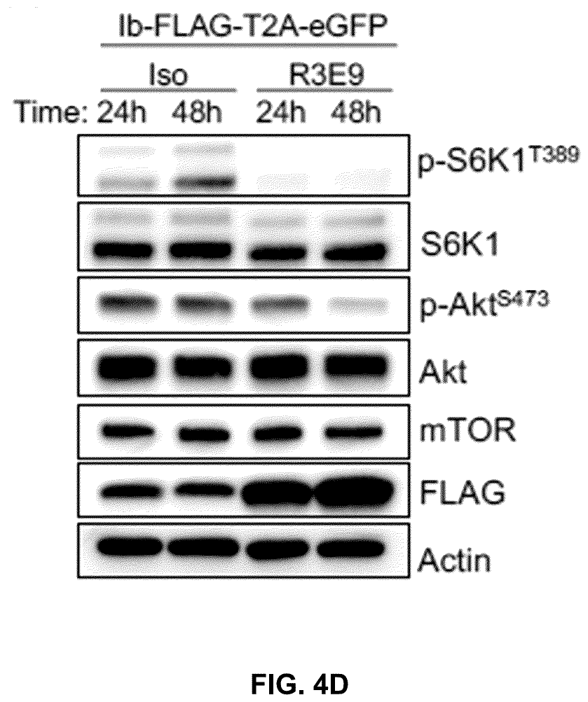

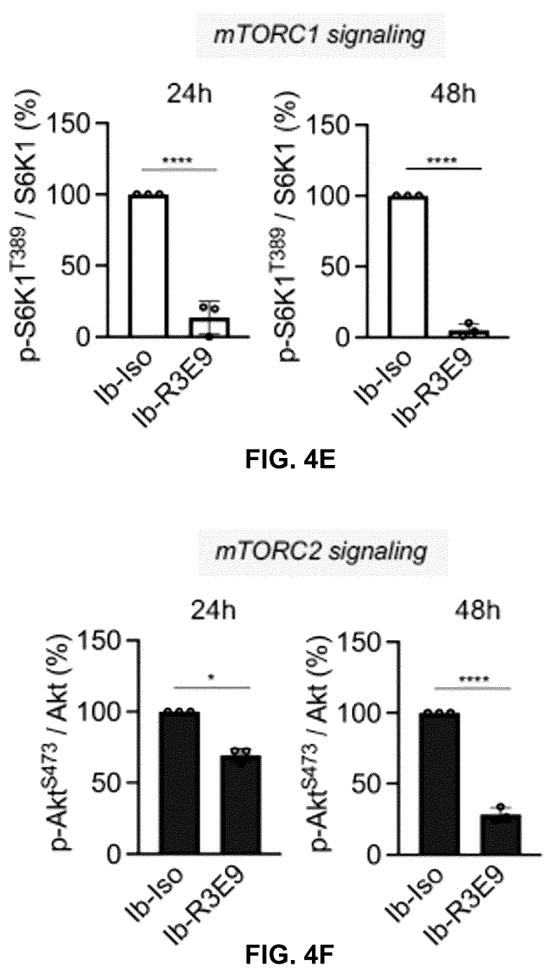

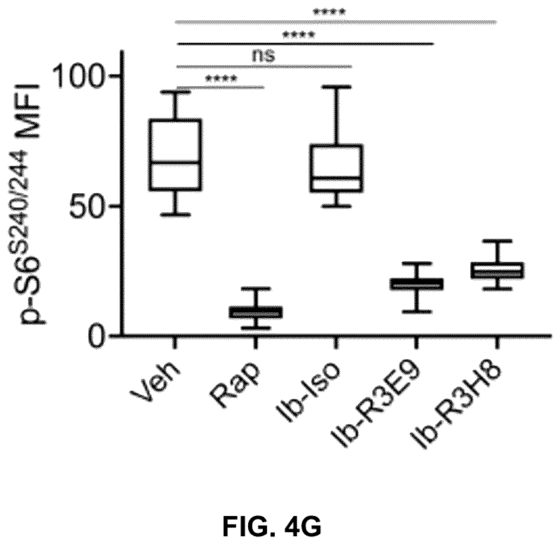

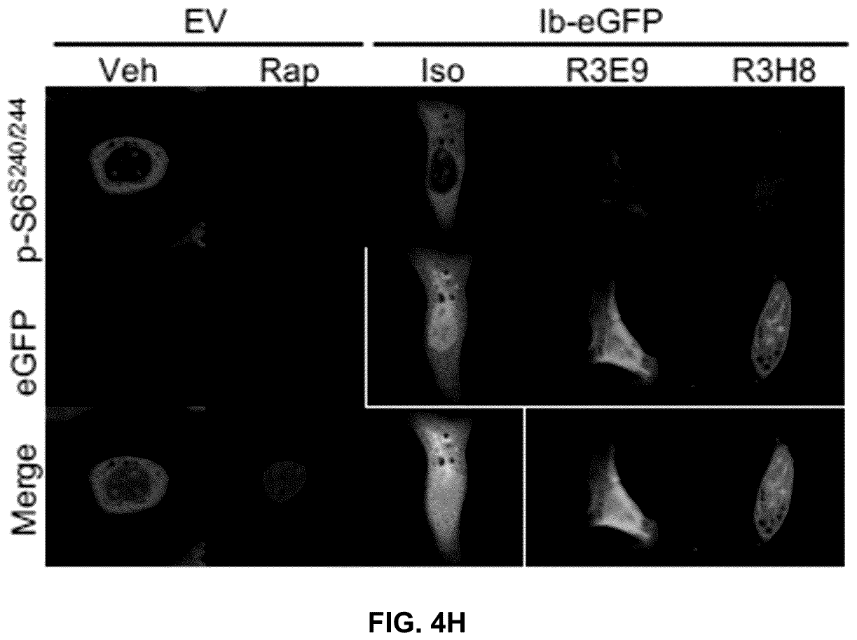

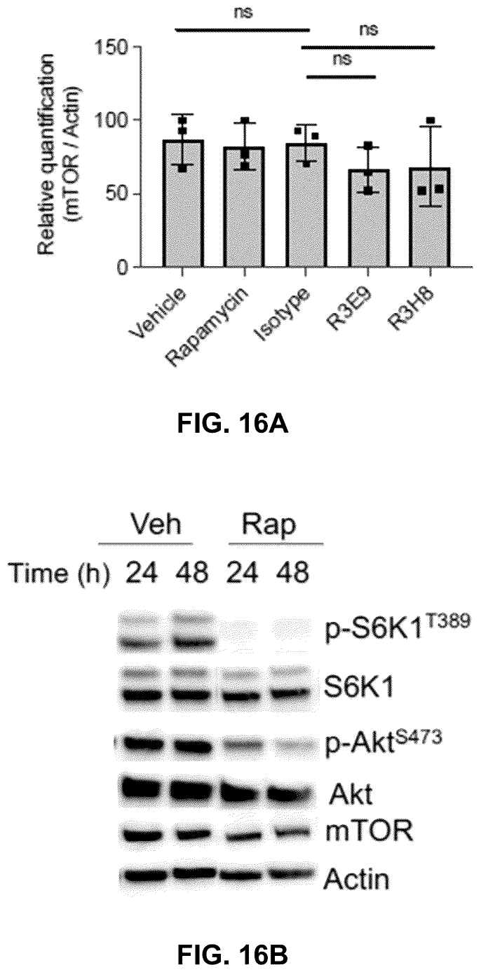

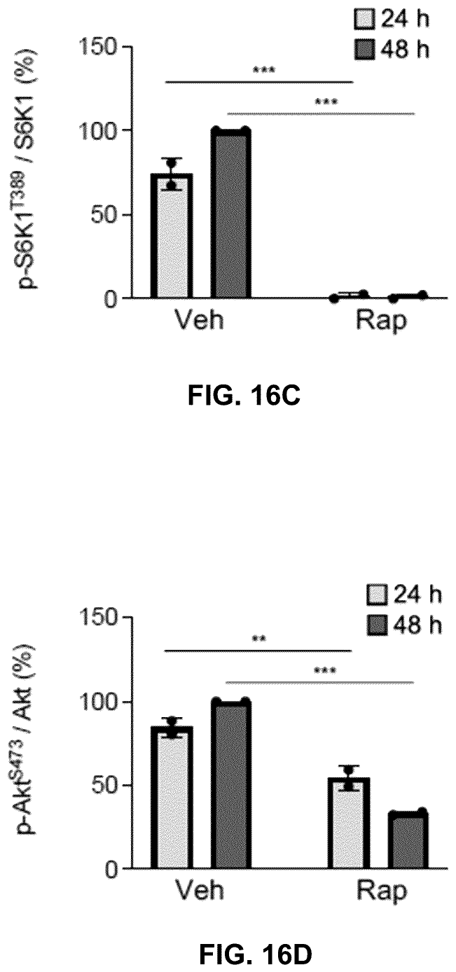

A fluorescence-activated cell sorting (FACS)-based screening approach was utilized to test the hypothesis that intrabody engagement of the mTOR^{FRB} substrate recruitment site can inhibit mTOR^{FRB}-dependent mTORC1 signaling. Western blotting analyzed phosphorylation levels of critical mTOR substrates in total Expi293F cell lysates from vehicle-treated cells, rapamycin-treated cells, and FACS-eGFP(+) cells expressing Ib-Iso, Ib-R3E9, or Ib-R3H8 (FIG. 4A). Both Ib-R3E9 and Ib-R3H8 effectively inhibited mTORC1-mediated phosphorylation of the rapamycin-sensitive S6K1^{T389} site without altering total mTOR levels (FIGS. 4B-4C and 16A).

Intrabody-mediated effects on mTORC1 and mTORC2 signaling were further investigated over different time scales by comparing them to rapamycin treatment. Ib-R3E9 robustly inhibited mTORC1-mediated phosphorylation of S6K1^{T389} after both 24 and 48 hours. In contrast, mTORC2-mediated phosphorylation of Akt^{S473} was only minimally suppressed after 24 hours but more prominently after 48 hours (FIG. 4D-4F). This pattern mirrored the time course observed in rapamycin-treated cells (FIG. 16B-16D). While accurate time scales of intrabody-based inhibition warrant further study, this was not feasible with the transfection-based screening approach used here.

Confocal microscopy was employed to visually inspect immunostained phosphorylation of ribosomal protein S6^{S240/244} levels as a reporter of mTORC1 inhibition in HeLa cells expressing eGFP-tagged Ib-Iso, Ib-R3E9, and Ib-R3H8. The localization of eGFP-fused intrabodies appeared diffuse throughout the cytosol and nucleus, consistent with previous reports of scFv-based intrabodies (FIG. 4G) (51). Intrabody-based mTORC1 inhibition efficacy was supported by the ablation of S6^{S240/244} phosphorylation levels in single cells transfected with Ib-R3E9 and Ib-R3H8 compared to the negative control Ib-Iso (FIG. 4H). These results underscore the capability of synthetic intrabodies to effectively inhibit mTORC1 and mTORC2 signaling in a genetically encoded manner similar to rapamycin-based inhibition (FIG. 4I).

Programmable Subcellular Intrabody Localization Enables Spatially Restricted Inhibition of mTOR in the Nucleus or Cytosol.

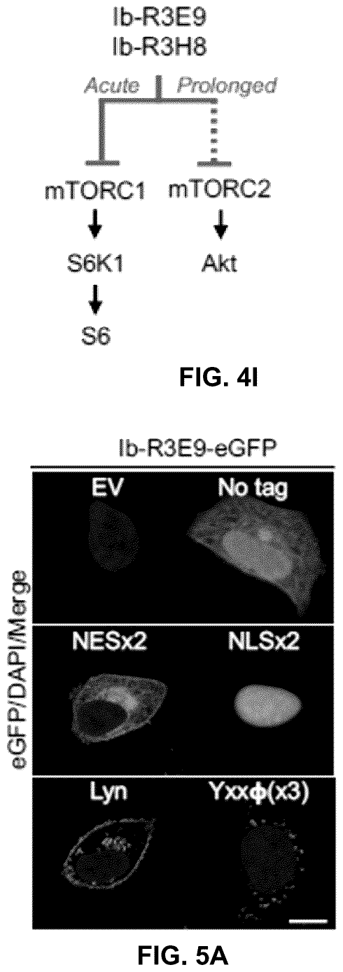

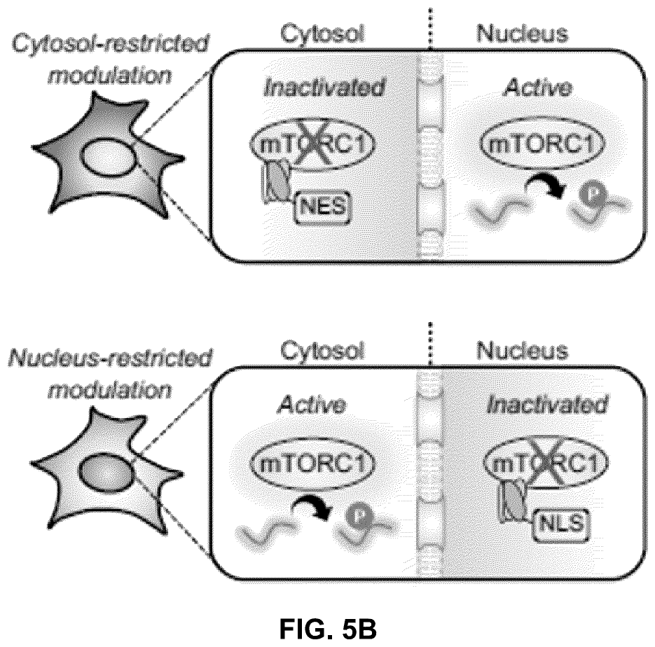

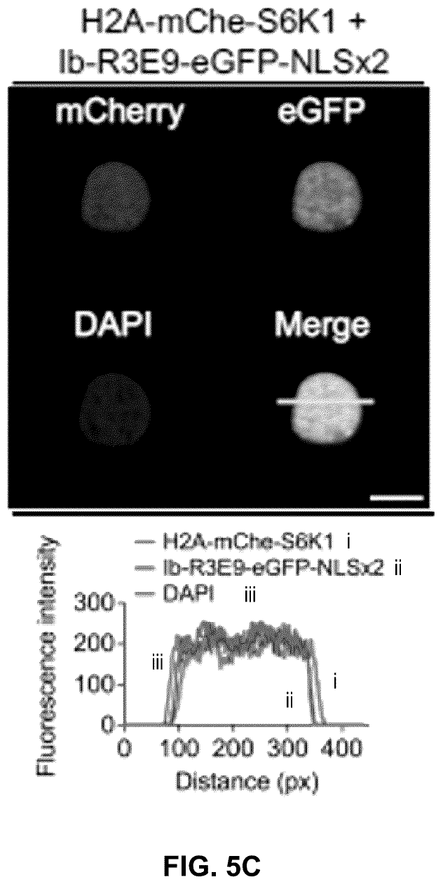

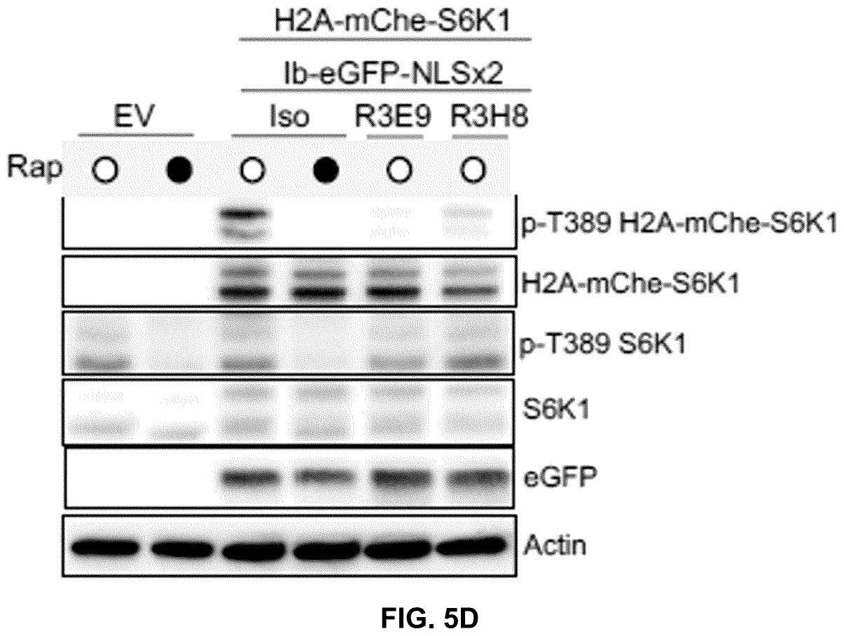

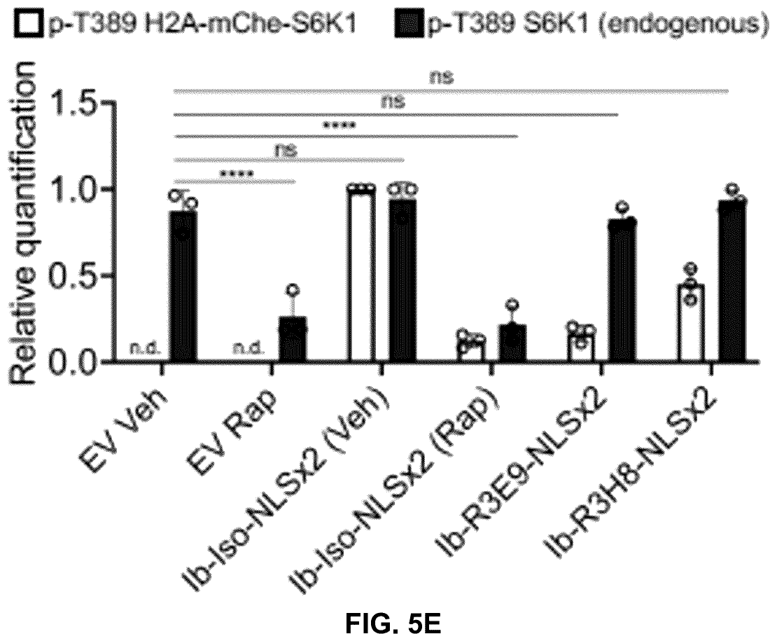

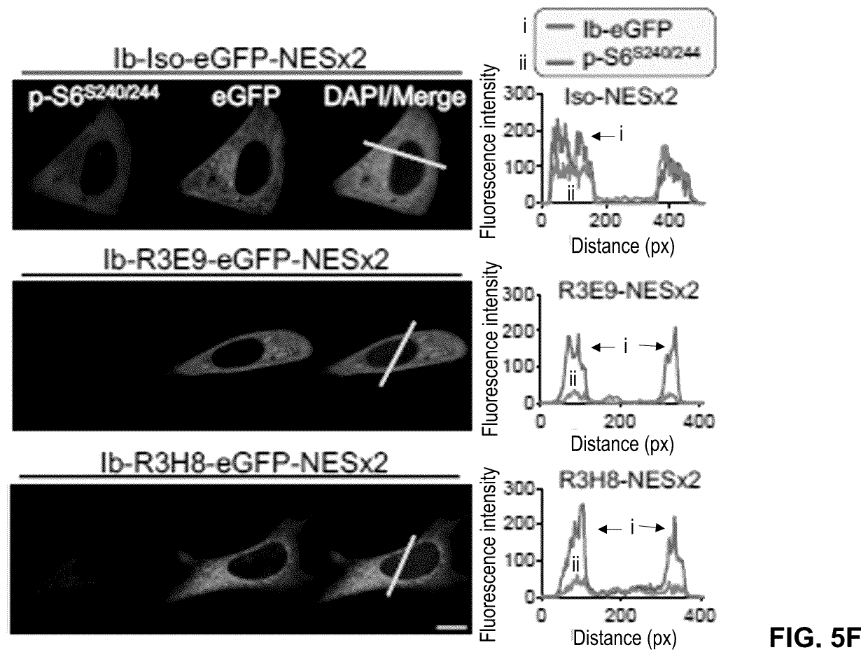

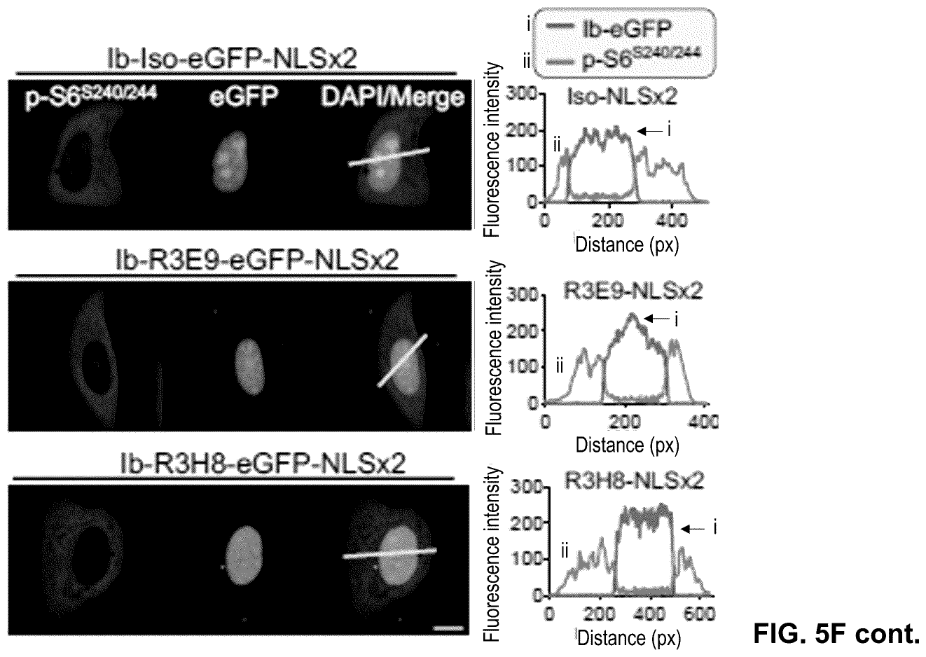

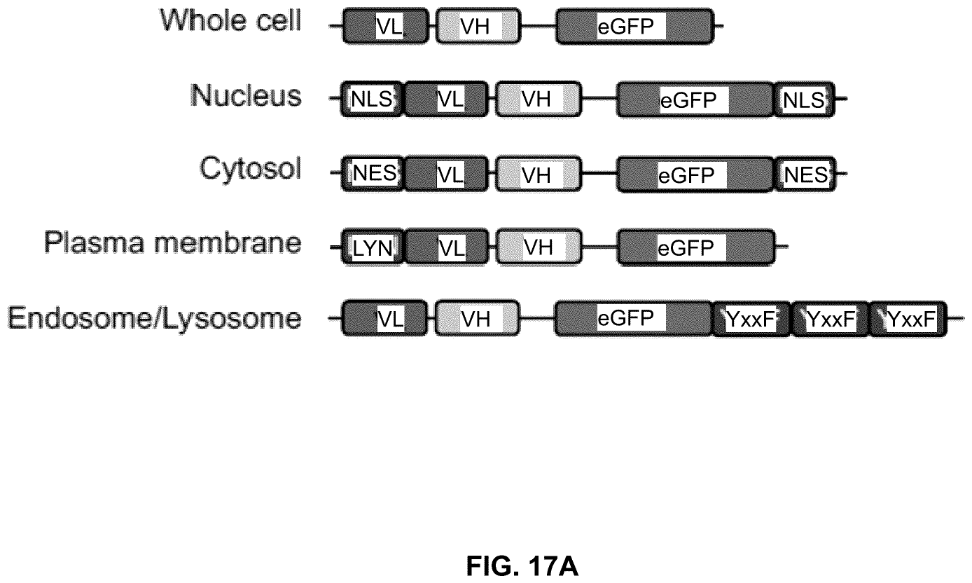

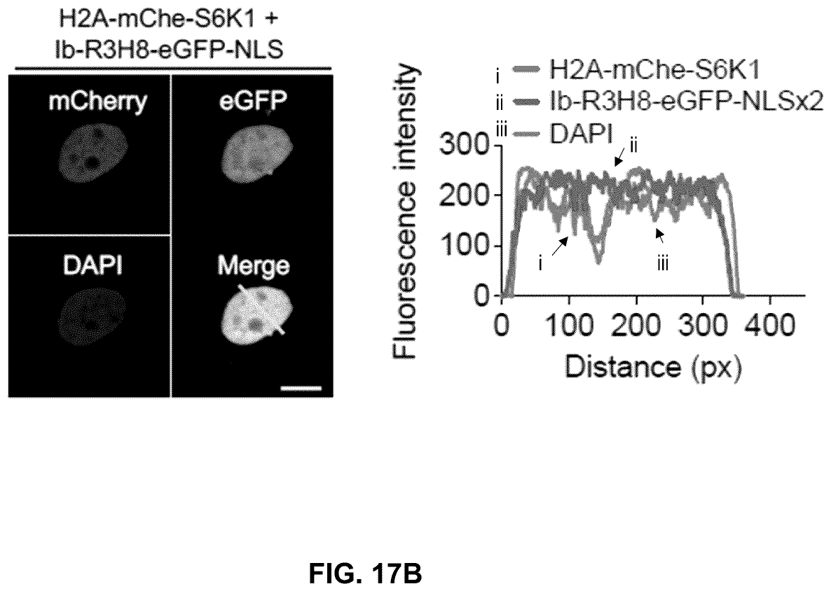

While small molecule inhibitors and genetic knockdown approaches have been invaluable for gaining general mechanistic insights into mTOR function, a novel platform was developed to perturb mTOR activity with subcellular spatial resolution. With structural and functional data supporting the efficacy of intrabody-based mTOR inhibition, Ib-R3E9 and Ib-R3H8 were modified by incorporating various subcellular localization tags (FIG. 17A). Transfection of Ib-R3E9-eGFP equipped with tags such as nuclear export signal (NES), nuclear localization signal (NLS), Lyn kinase motif, or Yxxφ motif in HeLa cells demonstrated the feasibility of directing synthetic intrabody inhibitors to specific locations within living cells (FIG. 5A).

The potential link between nuclear mTOR activity and distinct biological outputs is controversial due to the lack of tools for subcellular location-specific mTOR perturbations. Restricting intrabody residence to the cytosol or nucleus was envisioned as a means to selectively manipulate nuclear mTOR signaling separately from cytosolic mTOR signaling (FIG. 5B). To determine if nucleus-restricted intrabodies could selectively perturb nuclear mTOR signaling, the canonical mTORC1 substrate S6K1 was tagged with N-terminal histone 2A (H2A) and mCherry (mChe) for stringent nuclear anchoring as a readout for nuclear mTOR activity as previously described (29). Confocal microscopy of HeLa cells co-transfected with H2A-mChe-S6K1 and Ib-R3E9-eGFP-NLSx2 or Ib-R3H8-eGFP-NLSx2 showed clear nuclear colocalization for each construct (FIGS. 5C and 17B). Western blot analysis confirmed successful perturbation of nuclear mTOR activity, as indicated by phosphorylation of exogenous H2A-mChe-S6K1^{T389}, which was ablated by rapamycin treatment and expression of Ib-R3E9-eGFP-NLSx2 or Ib-R3H8-eGFP-NLSx2 (FIG. 5D). Importantly, expression of nucleus-restricted intrabodies did not inhibit phosphorylation of endogenous S6K1^{T389}, which served as a cytosolic control since it is not found at appreciable levels in the nucleus (FIG. 5E).

As an orthogonal validation, immunostained S6^{S240/244} phosphorylation was visualized as a reporter of the endogenous mTORC1 signaling network in response to cytosol-restricted or nucleus-restricted intrabody localization. S6^{S240/244} phosphorylation was predominantly cytosolic and was selectively ablated by expression of cytosolic Ib-R3E9-eGFP-NESx2 and Ib-R3H8-eGFP-NESx2 (FIG. 5F). In contrast, expression of nuclear Ib-R3E9-eGFP-NLSx2 and Ib-R3H8-eGFP-NLSx2 did not affect S6^{S240/244} phosphorylation levels, confirming that mTORC1 signaling remained unaffected in regions disconnected from the compartmentalized intrabodies. These results demonstrate the versatility of a modular intrabody-based system capable of perturbing mTOR signaling at specific subcellular locations.

### DISCUSSION

Genetically encodable probes have become essential tools for identifying druggable targets within complex therapeutic pathways. Despite advancements in fluorescence-based mTOR activity reporters and chemical-genetic mTOR inhibition platforms (20, 27, 28, 55), these methods would benefit significantly from the precise spatial and temporal control of mTOR signaling at the subcellular level. To enable this, the inventors developed genetically encodable synthetic binders for detailed analysis of subcellular mTOR signaling dynamics. The inventors engineered versatile scFv-based intrabodies that obstruct an important substrate recruitment site and stabilize specific conformational states of mTOR by recognizing nearly identical epitopes. These intrabodies enable subcellular activity perturbations in the mTOR signaling network with high spatial resolution, offering a novel approach to explore the structural and location-specific intricacies of the mTOR pathway.

A more complete understanding of the recruitment and stabilization of mTORC1 substrates for phosphorylation is crucial for developing new therapeutic strategies. The structure-guided mutational studies revealed significant similarities in how endogenous (e.g., S6K1 and PRAS40) and synthetic (e.g., sAB-R3E9 and sAB-R3H8) binding molecules recognize the mTOR^{FRB} substrate recruitment site. Despite low sequence identity at consensus interaction sites, hydrophobic shape complementarity appears to be a key factor in substrate docking. The mTOR^{FRB} site may also recruit other substrates like GRB10, TFEB, MAF1, and LIPIN through hydrophobic residues near phosphorylation sites (43). Understanding these interactions could provide insights into the molecular code of mTOR^{FRB} substrate coordination. Additionally, it remains unclear if nuclear mTORC1 substrates use this recruitment mechanism. Other mechanisms, such as TOS recruitment by Raptor, are vital for mTORC1 function. Developing intrabodies to block TOS-Raptor interactions will offer new ways to control mTOR function.

Engineered sABs are endowed with the ability to stabilize specific conformational states of their target antigens linked to biological functions. By combining high-resolution crystal structure analysis with cell-based functional assays, the inventors gained insights into the allosteric regulation of mTORC1 architecture. The results indicate that Ib-R3E9 and Ib-R3H8 stabilize distinct conformational states through coordination of the mTOR^{FRB} site. The inventors identified a novel high-energy conformation stabilized by sAB-R3E9, suggesting that mTOR^{FRB} might act as a conformational switch to regulate mTORC1 stability. The relevance of these conformational changes to nutrient-dependent signaling or kinase activity remains to be answered.

The inventors demonstrated effective rapamycin-like inhibition of mTOR by encoding Ib-R3E9 and Ib-R3H8 in human cell lines. This mode of inhibition is informative as it captures both therapeutic benefits and toxicities similar to rapamycin treatment. Notably, Ib-R3E9 showed near-complete inhibition of the rapamycin-sensitive p-S6K1^{T389} site, while Ib-R3H8 was slightly less potent, possibly due to lower intracellular solubility. The inventors also found that cytosolic and nuclear mTOR networks could be independently manipulated by tagging Ib-R3E9 and Ib-R3H8 with NES or NLS sequences. This provides a basis for studying how subcellular origins of mTOR signaling relate to biological functions, such as in prostate cancer where hyperactivation reprograms transcription through downstream components.

While cell culture models offer insights into signal transduction pathways, they don't fully capture organismal cellular communication complexities. Although the inventors haven't encoded intrabodies in model organisms yet, they could potentially provide spatial and temporal control over in vivo mTOR/TOR signaling for tissue- or cell-specific studies. The inventors demonstrated cross-reactivity with mTOR/TOR/TOR1 from various species using in vitro assays, suggesting that an epitope-directed engineering approach could yield selective binders for other model organisms. Overall, the intrabody-based platform should enable researchers to explore noncanonical mechanisms of subcellular mTOR signaling through systematic location-specific perturbations.

### Materials and Methods

Cell culture and transfections. Expi293F cells (Thermo Fisher Scientific) were cultured in Expi293 Expression Medium and handled according to the manufacturer's recommendations. Transfections in Expi293F were performed using the ExpiFectamine 293 Transfection Kit (Thermo Fisher Scientific) following the manufacturer's protocol except for 24-48 hours. The step of adding transfection enhancers 1 and 2 was disregarded here. HeLa cells (ATCC) were cultured in Dulbecco's modified Eagle medium (DMEM) supplemented with 1% Pen-Strep (Gibco) and 10% fetal bovine serum. Transfections in HeLa were performed using Lipofectamine LTX Reagent with PLUS Reagent (Thermo Fisher Scientific) according to the manufacturer's protocol for 24-48 hours.

Fluorescence-activated cell sorting. Expi293F cells transiently expressing FLAG-T2A-eGFP tagged intrabodies for the indicated time points were passed through 40 μm cell strainers (Falcon) and sorted using the BigFoot Cell Sorter (2-4 million cells each) into pre-warmed Expi293 Expression Medium. Untransfected Expi293F cells were used as a negative control to establish gates for sorting eGFP(+) cells. After sorting, cells were centrifuged at 200×g for 5 minutes, resuspended in pre-warmed Expi293 Expression Medium, and cultured at 37° C. for one hour before harvesting for immunoprecipitation or western blot analysis.

Protein expression and purification. Expression of SNAP-6×His, SNAP-mTOR^{FRB}-6×His, SNAP-FKBP12-6×His, and 10×His-TEV-mTOR^{FRB} was performed using BL21(DE3) competent E. coli Cells were grown in 2×YT medium to OD600=0.6-0.8, 1 mM IPTG was added, and cells were incubated overnight at 20° C. before harvesting by centrifugation. Purification followed a previously described protocol (56). Removal of 10×His-tag was performed by incubated TEV protease with 10×His-TEV-mTOR^{FRB} at 1:50 ratio (w/w) overnight at 4° C. in the presence of 0.5 mM TCEP. Next day, samples were incubated with TALON resin (Takara Bio) for 1 hour with rotation at 4° C. before collecting the flow through for purity analysis by SDS-PAGE. Periplasmic expression of Fabs was performed in BL21 competent E. coli. Cells were grown in 2×YT medium to OD₆₀₀=0.6-0.8, 1 mM IPTG was added, and cells were incubated for 4 hours at 37° C. before harvesting by centrifugation. Fab purification was performed as previously described (57). Fabs with AviTag were expressed in CVB101 cells following the same protocol above with the exception of adding 50 μM biotin at the time of induction.

Phage display selection. Phage display selection was performed as previously described (57). Prior to library sorting, SNAP-mTOR^{FRB} was site-specifically biotinylated by incubation with SNAP-Biotin (NEB) in the presence of 0.3 mM TCEP for 30 min at 37° C. Biotinylated SNAP-mTOR^{FRB} (Bio-SNAP-mTOR^{FRB}) was immobilized onto Streptavidin MagneSphere Paramagnetic Particles (Promega) for five rounds of phage selection. In the first round, 1 μM of Bio-SNAP-mTOR^{FRB} was immobilized on 200 μL streptavidin magnetic beads and was incubated with 1 mL phage library (10¹⁰ CFU) for 1 hour at room temperature with gentle shaking. The beads were washed three times to remove nonspecific phage, added to log phase E. coli XL-1 blue cells (Stratagene), and incubated for 20 minutes at room temperature. Then, media containing 100 μg/mL ampicillin and 109 p.f.u./mL of M13K07 helper phage (NEB) was added for overnight phage amplification at 37° C. The amplified phage was precipitated by adding 20% PEG, 2.5 M NaCl at a 1:5 ratio for 20 minutes on ice. Before each round, the amplified phage pool underwent competitive and subtractive selection using non-biotinylated SNAP-tag only and empty streptavidin paramagnetic beads for 30 minutes with shaking to eliminate nonspecific binders. The final antigen concentration was dropped systematically from 1 μM to 2 nM from the first to the fifth round (200 nM second round, 50 nM third round, 20 nM fourth round, and 2 nM fifth round). After phage binding, the beads were subjected to five washing rounds with 0.5% BSA/PBST. The bound phages were eluted using 0.1 M glycine, pH 2.6, and neutralized with TRIS-HCl, pH 8.5. Then, the phage eluate was used for E. coli infection and phage amplification, as described above. Additional selection pressure using 1 μM of non-biotinylated SNAP-tag in all washes was applied to ensure specificity to mTOR^{FRB}. After the fourth and fifth rounds the infected cells were plated on ampicillin agar and 192 colonies were picked to produce phage clones for single-point phage ELISA assay. Clones demonstrating high specificity for mTOR^{FRB} were sequenced and reformatted into the RH2.2 expression vector.

Crystallization and structure determination. sAB-R3E9•mTOR^{FRB} and sAB-R3H8•mTOR^{FRB} complexes were purified in in 10 mM HEPES, pH 7.2, 150 mM NaCl by size exclusion chromatography (SEC) using a Sephadex 200 column. Evaluation of SEC purity was performed by analyzing fractions via SDS-PAGE. Pure complexes were concentrated to at least 10 mg/mL for crystallization screening facilitated by the Mosquito Crystal Robot (TTP Labtech). Crystallization conditions for the sAB-R3E9•mTOR^{FRB} complex were 0.2 M Calcium chloride dihydrate, 16% PEG 3350. Crystals were soaked in reservoir solution containing 20% PEG 400 as a cryoprotectant before being flash frozen in liquid nitrogen. Crystallization conditions for the sAB-R3H8•mTOR^{FRB} complex were 0.1 M HEPES, pH 7.5, 4% PEG 400, 2.2 M ammonium sulfate. Crystals were directly flash-frozen in liquid nitrogen for data collection. X-ray diffraction data sets were collected at the 23-ID-D beamline of the Advanced Photon Source at Argonne National Laboratory. Molecular replacement was used to solve crystal structures of sAB-R3E9•mTOR^{FRB} and sAB-R3H8•mTOR^{FRB} using structures of FRB (PDB: 1FAP) and Fab (PDB: 6U8C) in Phaser-MR (58). Models were refined using phenix.refine (59) and manually built using Coot (60) over iterative alternating cycles. All structural figures were generated using PyMOL. Coordinates have been deposited to the Protein Data Bank (9DBO and 9DL0).

Generation of DNA constructs. All PCR and ligation reactions described here were performed using the In-Fusion HD Cloning Kit (Takara Bio). gBlock gene fragments (IDT) encoding FKBP12 and the FRB domain of mTOR (H. sapiens and residues corresponding to 2021-2113 for D. melanogaster, C. elegans, and S. cerevisiae TOR1) were cloned into the SmaI site of the pEKD40 expression vector bearing C-terminal 6×His-tags as previously described (60). H. sapiens mTOR^{FRB} was also cloned into BamHI/XhoI sites of the pHFT2 expression vector with an N-terminal 10×His-tag and TEV cleavage site. Synthetic antibody fragment (Fab) PCR inserts were generated by PCR from unique phage clones and ligated into SalI/HindIII sites of the pRH2.2 expression vector (non-tagged) or into SphI sites of the pSFV4 expression vector (AviTag). Mutagenesis was performed according to the QuikChange Site-Directed Mutagenesis Kit (Agilent). Single chain variable fragments were generated by cloning a gBlock fragments encoding sAB-R3E9 or sAB-R3H8 VL and VH domains as previously described (61) into XbaI/BamHI sites of the pSCSTa expression vector. Unique VH domains were cloned into XhoI/BamHI sites and CDR-L3 was mutated by a combination of PCR and In-Fusion ligation. C-terminal FLAG tag, eGFP, or FLAG-T2A-eGFP tags were generated by PCR. Intrabody localization tags (NES [LPPLERLTL], c-myc NLS [PAAKRVKLD], SV40 NLS [PKKKRKV], N-terminal Lyn kinase motif [GCIKSKRKDKD], and Yxxφ [YATFYSGMYQRL]) were generated by a combination of PCR and In-Fusion ligation in each of the eGFP-tagged intrabody expression vectors. Lastly, a gBlock gene fragment encoding histone 2A (H2A), mCherry, and p85 S6K1 was cloned into XbaI/BamHI sites of the pSCSTa expression vector.

Enzyme-Linked Immunosorbent Assays (ELISA). For phage ELISA, SNAP-mTOR^{FRB} (50 nM) was directly immobilized for 30 minutes onto high binding 96-well microplates (Greiner Bio) followed by extensive blocking with 2% BSA for 1 hour. After 15 minutes of incubation with phage, wells were washed three times with 0.5% BSA/PBST and incubated with Protein L-HRP (Thermo Scientific) for 20 minutes. The plates were again washed and developed with TMB substrate (Thermo Scientific) followed by quenching with 10% H₃PO₄ and absorbance measurement at 450 nm. For Fab-format ELISA, targets (200 nM) were directly immobilized onto high binding 96-well microplates (Greiner Bio). The wash buffer for all Fab-format experiments was PBS supplemented with 0.05% Tween 20. Plates were washed and blocked with 1% BSA in PBS. For competitive ELISA, competitors alone were incubated first for 15 minutes. Dilutions of Fabs alone or with competitors were prepared in PBS/0.05% Tween 20/0.5% BSA and were added for 20 minutes before washing three times. Secondary detection was carried out by Protein L-HRP (Thermo Scientific) for 20 minutes. Plates were washed three times before adding TMB substrate (Thermo Scientific), quenching with 10% H₃PO₄, and measuring absorbance at 450 nm.

Surface plasmon resonance. The MASS-1 (Bruker) instrument was used for all surface plasmon resonance (SPR) analyses. Ni-NTA sensor surface was used to immobilize mTOR^{FRB} via a 6×His-tag. Fabs in two-fold dilutions were run as analytes at 30 μl/min flow rate (20° C.). Raw data were corrected by double referencing. Analysis was performed with Sierra Analyser (Bruker) using a Langmuir 1:1 binding model for curve fitting. Results were then plotted using GraphPad Prism.

In vitro pulldown assay. SNAP-FKBP12 was site-specifically labeled with SNAP-Surface Alexa Fluor 488 (NEB) according to the manufacturer's protocol. The dilution and wash buffer for all pulldowns was PBS supplemented with 0.05% Tween 20 and 0.5% BSA. All incubations were performed in the dark at room temperature. Briefly, 10 nM Bio-SNAP-mTOR^{FRB}, 10 nM A488-SNAP-FKBP12, and 10 nM rapamycin were incubated together briefly to allow ternary complex formation. Varying concentrations of sAB-R3E9 or sAB-R3H8 were spiked into each sample and incubated 30 minutes. Next, 10 μL of Streptavidin MagneSphere Paramagnetic Particles (Promega) were added to each samples and incubated with constant rotation for 15 mins. Samples were washed three times in 0.3 mL wash buffer supplemented with 10 nM rapamycin before elution by heating 5 mins in 20 μL 1× sample buffer for SDS-PAGE analysis. A488-SNAP-FKBP12 levels were quantified using Image Lab software to determine IC₅₀ values for sAB-R3E9 and sAB-R3H8.

Immunoprecipitation and western blot analysis. For experiments with cells transfected with H2A-mChe-S6K1, the lysis buffer used was 1×RIPA (EMD Millipore) supplemented with 0.1% sodium dodecyl sulfate and 1×Halt Protease Inhibitor Cocktail (Thermo Scientific). For all other experiments, to maintain the integrity of mTORC1 assemblies, the lysis buffer used was 0.3% CHAP, 25 mM TRIS, pH 8.0, 150 mM NaCl supplemented with 5 mM EDTA and 1×Halt Protease Inhibitor Cocktail. Briefly, cells were washed one time with ice cold PBS. HeLa cells were collected by gentle scraping in ice cold PBS followed by centrifugation. Cell lysis was carried out on ice for 20 minutes with constant agitation before centrifugation at 14,000 rpm to clarify lysates. Supernatants were transferred to fresh tubes and total protein was quantified by BCA assay (Thermo Scientific). Biotinylated sAB immunoprecipitations were performed for 3 hours with rotation at 4° C. Dynabeads M-270 Streptavidin beads (Invitrogen) were equilibrated in lysis buffer before adding to lysates for 1 hour with rotation at 4° C. Beads were washed five times in lysis buffer before elution by boiling 5 minutes in SDS sample buffer containing 10 mM DTT. Anti-FLAG immunoprecipitations were performed by adding 30 μL of Anti-DYKDDDDK G1 Affinity Resin (Genscript) to lysates for 3 hours with rotation at 4° C. Samples were washed five times in lysis buffer before elution by boiling 5 minutes in SDS sample buffer. Samples were adjusted to 10 mM DTT after transferring supernatant to fresh tubes. Pan-mTOR immunoprecipitations were performed by adding anti-mTOR Rabbit IgG (Cell Signaling #2972, 1:200 dilution) to lysates and incubating overnight with rotation at 4° C. The next day, Protein A Magnetic Beads (Pierce) were equilibrated in lysis buffer and added for 1 hour with rotation at 4° C. Beads were washed five times in lysis buffer before elution by boiling 5 minutes in SDS sample buffer containing 10 mM DTT. Samples were separated via SDS-PAGE at 100V, transferred to Immobilin-P PVDF Membranes (0.45 μm, EMI DMillipore), and blocked for 1 hour at room temperature in blocking buffer (5% BSA in PBS supplemented with 0.1% Tween 20). The following primary antibodies were obtained from Cell Signaling Technology: mTOR (#2972S), Raptor (#2280S), mLST8 (#3274S), Rictor (#2114S), p-S6K1^{T389} (#9234S), S6K1 (#2708T), p-Akt^{S473} (#9271T), Akt (#4691T), Actin (#4970T), Tubulin (#2148S), FLAG (#14793S), and eGFP (#2956T). Antibodies were diluted in blocking buffer and incubated with membranes overnight at 4° C. with gentle rocking. Membranes were washed three to four times for five minutes each in wash buffer (PBS supplemented with 0.1% Tween 20) before adding Anti-Rabbit IgG, HRP-Linked Antibody (Cell Signaling Technology, #7074P2) for one hour at room temperature in blocking buffer. Membranes were washed three to four times for five minutes each in wash buffer and then developed using SuperSignal West Pico PLUS Chemiluminescent Substrate (Thermo Fisher Scientific).

Immunofluorescent staining and confocal microscopy imaging. HeLa cells were seeded onto ibiTreat μslide 8-Well slides (Ibidi) and grown in Dulbecco's modified Eagle medium (DMEM) supplemented with 1% Pen-Strep (Gibco) and 10% fetal bovine serum until they reached 80-90% confluency before transfection or immunostaining. Imaging was performed using a Stellaris 8 (Leica) confocal microscope. Quantitative analyses were performed using FIJI (62). For mTOR/LAMP1 co-localization immunostaining, HeLa cells were washed once with PBS before fixation for 15 minutes at room temperature in 4% paraformaldehyde. Cells were washed three times in PBS and then permeabilized with 0.2% Triton X-100 for 10 minutes followed by three washes in PBS. Blocking was performed by adding 3% BSA, 0.2% Triton X-100 for 1 hour at room temperature. sAB-R3E9 and anti-LAMP1 (Cell Signaling Technology, #9091T) antibodies were diluted in 1% BSA, 0.2% Triton X-100 and added for 2 hours at 4° C. Cells were washed three times with PBS. Goat Anti-Human IgG, F(ab′)₂ fragment specific conjugated with Alexa647 and Goat Anti-Rabbit IgG (H+L) conjugated with Alexa488 (Jackson ImmunoResearch) were diluted in 1% BSA, 0.2% Triton X-100 and added for 45 minutes at room temperature. Cells were washed three times with PBS and incubated with 1 μg/μL DAPI (Thermo Fisher Scientific) for 10 minutes before washing again three times with PBS. PBS in 50% glycerol was added as the final step. For p-S6^{S240/244} (Cell Signaling Technology, #5364) immunostaining, all steps were performed the same as above with the following exceptions: (i) HeLa cells were first transfected according to the manufacturers protocol, (ii) p-S6^{S240/244} Rabbit IgG was added overnight at at 4° C., and (iii) Goat Anti-Rabbit IgG (H+L) conjugated with Alexa647 (Jackson ImmunoResearch) was used for secondary detection.

TABLE S1

| | sAB-R3H8•mTOR^{FRB} | sAB-R3E9•mTOR^{FRB} |
|---|---|---|
| | (PDB 9DL0) | (PDB 9DBO) |
| Wavelength (Å) | 1.0332 | 1.0332 |
| Space group | P 21 2 21 | P 1 2 1 |

Cell dimensions

| a, b, c (Å) | 87.613, 110.042, 131.079, | | 45.055, 69.732, 89.413 | |
|---|---|---|---|---|
| α, β, γ (°) | 90, 90, 90 | | 90, 90.12, 90 | |
| Resolution (Å) | 56.31-2.0 | (2.02-2.0)* | 45.05-1.55 | (1.57-1.55) |
| Rmerge | 0.054 | (0.955) | 0.056 | (0.671) |
| Rmeas | 0.076 | (1.351) | 0.079 | (0.949) |
| Rpim | 0.054 | (0.955) | 0.056 | (0.671) |
| CC½ | 0.998 | (0.295) | 0.994 | (0.475) |
| I/σI | 8.8 | (1.0) | 6.8 | (1.0) |
| Completeness (%) | 94.32 | (99.75) | 93.9 | (71.0) |
| Redundancy | 2.0 | (2.0) | 1.9 | (1.8) |

| | | |
|---|---|---|
| No. reflections | 81,284 | 75,225 |
| Rwork/Rfree | 0.2050/0.2353 | 0.2043/0.2338 |

No. atoms

| | | |
|---|---|---|
| Protein | 8113 | 4099 |
| Solvent | 373 | 548 |
| Protein residues | 1056 | 529 |

R.M.S deviations

| | | |
|---|---|---|
| Bond lengths (Å) | 0.008 | 0.008 |
| Bond angles (°) | 0.99 | 1.1 |
| Ramachandran | 97.41 | 97.9 |
| favored (%) | | |
| Allowed (%) | 2.59 | 2.1 |
| Outliers (%) | 0 | 0 |
| Rotamer | 1.22 | 0.44 |
| outliers (%) | | |
| Clashscore | 2.71 | 1.98 |

Average B-factor

| | | |
|---|---|---|
| Protein | 46.18 | 14.66 |
| Solvent | 45.02 | 34.05 |

### Example 2: Key Findings and Contributions of Certain Aspects Described Herein

### 1. Engineering Synthetic Intrabodies

The inventors developed synthetic antibody fragments (Fabs) targeting the FKBP12-Rapamycin binding (FRB) domain of mTOR, a key site for substrate recruitment.

Using phage display techniques, they identified high-affinity binders that selectively obstruct the substrate recruitment site of mTORC1, mimicking the natural product rapamycin.

Two intrabodies examples, sAB-R3E9 and sAB-R3H8, were characterized for their ability to interact with distinct conformational states of mTOR's FRB domain.

### 2. Structural Insights

High-resolution crystal structures of the intrabodies bound to mTOR revealed how they mimic natural substrates like S6K1 and PRAS40 by engaging hydrophobic residues at key positions.

These structures provided insights into the allosteric regulation of mTORC1 stability, showing that subtle conformational changes in the FRB domain can influence complex assembly and activity.

3. Functional Modulation of mTOR Signaling

Intrabodies were expressed in human cells to study their effects on mTOR signaling. Both sAB-R3E9 and sAB-R3H8 inhibited phosphorylation of mTORC1 substrates such as S6K1 (at T389), similar to rapamycin.

The intrabodies also differentially affected the stability of the mTOR-Raptor interaction, a key component of mTORC1. For instance, Ib-R3H8 destabilized this interaction more than Ib-R3E9.

4. Spatial Control of mTOR Activity

The study demonstrated that intrabody localization could be genetically programmed using nuclear localization signals (NLS) or nuclear export signals (NES), enabling compartment-specific inhibition.

Cytosol-restricted intrabodies inhibited cytosolic mTORC1 signaling, while nucleus-restricted intrabodies selectively perturbed nuclear mTOR activity without affecting cytosolic functions.

### 5. Applications

Aspects described herein can allow for precise dissection of subcellular mTOR signaling networks and can be used to explore how spatially distinct mTOR activities contribute to diseases like cancer or metabolic disorders. The developments described herein establish intrabodies as versatile tools for dissecting protein kinase networks, providing a framework for exploring structural and spatial mechanisms in cellular signaling pathways. This could significantly enhance our understanding of complex signaling networks and their role in disease progression. Overall, this work represents a significant advancement in the ability to study and manipulate intracellular signaling pathways with high spatial precision, paving the way for more targeted therapeutic interventions.

The approach offers a new avenue for developing targeted therapies that minimize systemic side effects associated with traditional mTOR inhibitors. By offering a method to selectively modulate mTOR signaling with spatial resolution, the study opens new avenues for therapeutic strategies that target specific cellular compartments, potentially reducing side effects associated with systemic mTOR inhibition. This approach is particularly relevant for diseases like cancer and metabolic disorders where dysregulated mTOR activity is implicated.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, and those cited elsewhere herein, to the extent that they provide exemplary procedural or other details supplementary to those set forth herein, are specifically incorporated herein by reference.
1. R. A. Saxton, D. M. Sabatini, mTOR Signaling in Growth, Metabolism, and Disease. Cell 168, 960-976 (2017).
2. J. J. Howell, B. D. Manning, mTOR couples cellular nutrient sensing to organismal metabolic homeostasis. Trends Endocrinol Metab 22, 94-102 (2011).
3. E. Audet-Walsh, et al., Nuclear mTOR acts as a transcriptional integrator of the androgen signaling pathway in prostate cancer. Genes Dev 31, 1228-1242 (2017).
4. Y. Zhong, X. Zhou, K.-L. Guan, J. Zhang, Rheb regulates nuclear mTORC1 activity independent of farnesylation. Cell Chemical Biology 29, 1037-1045.e4 (2022).
5. D.-H. Kim, et al., mTOR interacts with raptor to form a nutrient-sensitive complex that signals to the cell growth machinery. Cell 110, 163-175 (2002).
6. D. D. Sarbassov, et al., Rictor, a novel binding partner of mTOR, defines a rapamycin-insensitive and raptor-independent pathway that regulates the cytoskeleton. Curr Biol 14, 1296-1302 (2004).
7. A. Jain, et al., Stoichiometry and assembly of mTOR complexes revealed by single-molecule pulldown. Proc Natl Acad Sci USA 111, 17833-17838 (2014).
8. S. Battaglioni, D. Benjamin, M. Wälchli, T. Maier, M. N. Hall, mTOR substrate phosphorylation in growth control. Cell 185, 1814-1836 (2022).
9. D. E. Harrison, et al., Rapamycin fed late in life extends lifespan in genetically heterogeneous mice. Nature 460, 392-395 (2009).
10. A. V. Shindyapina, et al., Rapamycin treatment during development extends life span and health span of male mice and Daphnia magna. Science Advances 8, eabo5482.
11. P. Juricic, et al., Long-lasting geroprotection from brief rapamycin treatment in early adulthood by persistently increased intestinal autophagy. Nature Aging 2, 824-836 (2022).
12. J. M. Schinaman, A. Rana, W. W. Ja, R. I. Clark, D. W. Walker, Rapamycin modulates tissue aging and lifespan independently of the gut microbiota in Drosophila. Scientific Reports 9, 7824 (2019).
13. J. R. Roth, et al., Rapamycin reduces neuronal mutant huntingtin aggregation and ameliorates locomotor performance in Drosophila. Frontiers in Aging Neuroscience 15 (2023).
14. S. Robida-Stubbs, et al., TOR signaling and rapamycin influence longevity by regulating SKN-1/Nrf and DAF-16/FoxO. Cell Metab 15, 713-724 (2012).
15. L. Willems, et al., The dual mTORC1 and mTORC2 inhibitor AZD8055 has anti-tumor activity in acute myeloid leukemia. Leukemia 26, 1195-1202 (2012).
16. S.-Y. Sun, mTOR kinase inhibitors as potential cancer therapeutic drugs. Cancer Lett 340, 1-8 (2013).
17. K. E. O'Reilly, et al., mTOR inhibition induces upstream receptor tyrosine kinase signaling and activates Akt. Cancer Res 66, 1500-1508 (2006).
18. J. Liu, et al., Targeting the mTOR pathway in breast cancer. Tumour Biol 39, 1010428317710825 (2017).
19. S. A. Soefje, A. Karnad, A. J. Brenner, Common toxicities of mammalian target of rapamycin inhibitors. Target Oncol 6, 125-129 (2011).
20. D. R. Wassarman, K. Bankapalli, L. J. Pallanck, K. M. Shokat, Tissue-restricted inhibition of mTOR using chemical genetics. Proc Natl Acad Sci USA 119, e2204083119 (2022).
21. Z. Zhang, et al., Brain-restricted mTOR inhibition with binary pharmacology. Nature 609, 822-828 (2022).
22. D. Filer, et al., RNA polymerase III limits longevity downstream of TORC1. Nature 552, 263-267 (2017).
23. V. Giguère, Canonical signaling and nuclear activity of mTOR-a teamwork effort to regulate metabolism and cell growth. FEBS J 285, 1572-1588 (2018).
24. C. Betz, M. N. Hall, Where is mTOR and what is it doing there? J Cell Biol 203, 563-574 (2013).
25. J. M. Carosi, C. Fourrier, J. Bensalem, T. J. Sargeant, The mTOR-lysosome axis at the centre of ageing. FEBS Open Bio 12, 739-757 (2022).
26. K. B. Rogala, et al., Structural basis for the docking of mTORC1 on the lysosomal surface. Science 366, 468-475 (2019).
27. X. Zhou, et al., Dynamic Visualization of mTORC1 Activity in Living Cells. Cell Rep 10, 1767-1777 (2015).
28. N. Bouquier, et al., AIMTOR, a BRET biosensor for live imaging, reveals subcellular mTOR signaling and dysfunctions. BMC Biology 18, 81 (2020).
29. X. Zhou, et al., Location-specific inhibition of Akt reveals regulation of mTORC1 activity in the nucleus. Nat Commun 11, 6088 (2020).
30. C. K. Tsang, H. Liu, X. F. S. Zheng, mTOR binds to the promoters of RNA polymerase I- and III-transcribed genes. Cell Cycle 9, 953-957 (2010).
31. T. Kantidakis, B. A. Ramsbottom, J. L. Birch, S. N. Dowding, R. J. White, mTOR associates with TFIIIC, is found at tRNA and 5S rRNA genes, and targets their repressor Maf1. Proceedings of the National Academy of Sciences 107, 11823-11828 (2010).
32. C. R. Dufour, et al., The mTOR chromatin-bound interactome in prostate cancer. Cell Reports 38, 110534 (2022).
33. F. Artoni, N. Grutzmacher, C. Demetriades, Unbiased evaluation of rapamycin's specificity as an mTOR inhibitor. Aging Cell 22, e13888 (2023).
34. Y. Chen, L. Han, C. R. Dufour, A. Alfonso, V. Giguère, Canonical and Nuclear mTOR Specify Distinct Transcriptional Programs in Androgen-Dependent Prostate Cancer Cells. Molecular Cancer Research 22, 113-124 (2024).
35. E. S. Ali, et al., Recent advances and limitations of mTOR inhibitors in the treatment of cancer. Cancer Cell International 22, 284 (2022).
36. B. Mao, et al., Overview of Research into mTOR Inhibitors. Molecules 27 (2022).
37. J. Li, S. G. Kim, J. Blenis, Rapamycin: one drug, many effects. Cell Metab 19, 373-379 (2014).
38. J. Chen, X. F. Zheng, E. J. Brown, S. L. Schreiber, Identification of an 11-kDa FKBP12-rapamycin-binding domain within the 289-kDa FKBP12-rapamycin-associated protein and characterization of a critical serine residue. Proc Natl Acad Sci USA 92, 4947-4951 (1995).
39. P. P. Hsu, et al., The mTOR-regulated phosphoproteome reveals a mechanism of mTORC1-mediated inhibition of growth factor signaling. Science 332, 1317-1322 (2011).
40. S. A. Kang, et al., mTORC1 Phosphorylation Sites Encode Their Sensitivity to Starvation and Rapamycin. Science 341, 1236566 (2013).
41. C. Gaubitz, et al., Molecular Basis of the Rapamycin Insensitivity of Target Of Rapamycin Complex 2. Molecular Cell 58, 977-988 (2015).
42. H. Yang, et al., mTOR kinase structure, mechanism and regulation. Nature 497,217-223 (2013).
43. H. Yang, et al., Mechanisms of mTORC1 activation by RHEB and inhibition by PRAS40. Nature 552, 368-373 (2017).
44. A. Pini, L. Bracci, Phage display of antibody fragments. Curr Protein Pept Sci 1, 155-169 (2000).
45. M. Paduch, et al., Generating conformation-specific synthetic antibodies to trap proteins in selected functional states. Methods 60, 3-14 (2013).
46. P. Akkapeddi, et al., Exploring switch II pocket conformation of KRAS(G12D) with mutant-selective monobody inhibitors. Proc Natl Acad Sci USA 120, e2302485120 (2023).
47. A. Zorba, et al., Allosteric modulation of a human protein kinase with monobodies. Proc Natl Acad Sci USA 116, 13937-13942 (2019).
48. M. Baidya, et al., Allosteric modulation of GPCR-induced β-arrestin trafficking and signaling by a synthetic intrabody. Nature Communications 13, 4634 (2022).
49. E. Ghosh, et al., A synthetic intrabody-based selective and generic inhibitor of GPCR endocytosis. Nature Nanotechnology 12, 1190-1198 (2017).
50. H. Farrants, et al., Chemogenetic Control of Nanobodies. Nature Methods 17, 279-282 (2020).
51. L. J. Bailey, et al., Locking the Elbow: Improved Antibody Fab Fragments as Chaperones for Structure Determination. Journal of Molecular Biology 430, 337-347 (2018).
52. M. Baidya, et al., Genetically encoded intrabody sensors report the interaction and trafficking of β-arrestin 1 upon activation of G-protein-coupled receptors. Journal of Biological Chemistry 295, 10153-10167 (2020).
53. R. J. Petrella, M. Karplus, The energetics of off-rotamer protein side-chain conformations 11 Edited by F. Cohen. Journal of Molecular Biology 312, 1161-1175 (2001).
54. B. Chahkandi, M. Chahkandi, An accurate DFT study within conformational survey of the D-form serine-alanine protected dipeptide. BMC Chem 17, 138 (2023).
55. C. Li, et al., TORSEL, a 4EBP1-based mTORC1 live-cell sensor, reveals nutrient-sensing targeting by histone deacetylase inhibitors. Cell Biosci 14, 68 (2024).
56. T. Slezak, et al., Engineered Protein-G variants for plug-and-play applications. bioRxiv2024.08.06.606809 (2024). https://doi.org/10.1101/2024.08.06.606809.
57. T. Slezak, A. A. Kossiakoff, Engineered Ultra-High Affinity Synthetic Antibodies for SARS-CoV-2 Neutralization and Detection. J Mol Biol 433, 166956 (2021).
58. A. J. McCoy, et al., Phaser crystallographic software. J Appl Crystallogr 40, 658-674 (2007).
59. P. V. Afonine, et al., Towards automated crystallographic structure refinement with phenix.refine. Acta Crystallogr D Biol Crystallogr 68, 352-367 (2012). P. Emsley, K. Cowtan, Coot: model-building tools for molecular graphics. Acta Crystallogr D Biol Crystallogr 60, 2126-2132 (2004).
60. T. Slezak, et al., An engineered ultra-high affinity Fab-Protein G pair enables a modular antibody platform with multifunctional capability. Protein Sci 29, 141-156 (2020).
61. M. Baidya, et al., Genetically encoded intrabody sensors report the interaction and trafficking of β-arrestin 1 upon activation of G-protein-coupled receptors. Journal of Biological Chemistry 295, 10153-10167 (2020).
62. J. Schindelin, et al., Fiji: an open-source platform for biological-image analysis. Nat Methods 9, 676-682 (2012).

1. An antibody or antigen binding fragment comprising a heavy chain variable region and a light chain variable region, wherein the heavy chain variable region comprises a HCDR1, HCDR2, and HCDR3 having at least 80% sequence identity to the HCDR1, HCR2, HCR3 from a heavy chain variable region of an antibody clone of Table 1 and wherein the light chain variable region comprises a LCDR1, LCDR2, and LCDR3 having at least 80% sequence identity to the LCDR1, LCDR2, and LCDR3 from the light chain variable region of the same antibody clone of Table 1.

2. The antibody or antigen binding fragment of claim 1, wherein the heavy chain variable region comprises a HCDR1, HCDR2, and HCDR3 having the amino acid sequence of a HCDR1, HCDR2, and HCDR3 of a clone of Table 1 and wherein the light chain variable region comprises a LCDR1, LCDR2, and LCDR3 comprising the amino acid sequence of the LCDR1, LCDR2, and LCDR3 from the light chain variable region of the same clone of Table 1.

3-10. (canceled)

11. The antibody of claim 1, wherein the antibody is human, chimeric, or humanized.

12. The antibody or antigen-binding fragment of claim 1, wherein the antibody, or antigen binding fragment binds an FKBP12-Rapamycin binding domain of an mTOR protein with a KD between about 10-6 M/L and 10-12 M/L.

13. The antibody or antigen binding fragment of claim 1, wherein the antibody is a neutralizing antibody.

14. The antibody or antigen binding fragment of claim 1, wherein the antibody is a human antibody, humanized antibody, recombinant antibody, chimeric antibody, an antibody derivative, a veneered antibody, a diabody, a monoclonal antibody, a single domain antibody, or a single chain antibody.

15. The antigen binding fragment of claim 1, wherein the antigen binding fragment is a single chain variable fragment (scFv), F(ab′)2, Fab′, Fab, Fv, or rIgG.

16. The antibody or antigen binding fragment of claim 1, further comprising a subcellular localization domain.

17. (canceled)

18. A polypeptide comprising the antibody or antigen binding fragment of claim 1.

19-21. (canceled)

22. A composition comprising the antibody or antigen binding fragment of claim 1.

23. The composition of claim 22, wherein the composition comprises a pharmaceutical excipient.

24. The composition of claim 22, wherein the composition further comprises an adjuvant.

25. The composition of claim 22, wherein the composition is formulated for parenteral, intravenous, subcutaneous, intramuscular, or intranasal administration.

26. The composition of claim 22, wherein the composition comprises at least two antibodies or antigen binding fragments.

27. One or more nucleic acids encoding the antibody or antigen binding fragment of claim 1.

28. (canceled)

29. A host cell comprising the nucleic acid of claim 27.

30. (canceled)

31. A method of a making a cell comprising transferring the nucleic acid(s) of claim 27 into a cell.

32-34. (canceled)

35. A method for producing a polypeptide comprising transferring the nucleic acid(s) of claim 27 into a cell and isolating polypeptides expressed from the nucleic acid.

36. (canceled)

37. A method for treating or preventing a disease in a subject, the method comprising administering to the subject the antibody or antigen binding fragment comprising a heavy chain variable region and a light chain variable region, wherein the heavy chain variable region comprises a HCDR1, HCDR2, and HCDR3 having at least 80% sequence identity to the HCDR1, HCR2, HCR3 from a heavy chain variable region of an antibody clone of Table 1 and wherein the light chain variable region comprises a LCDR1, LCDR2, and LCDR3 having at least 80% sequence identity to the LCDR1, LCDR2, and LCDR3 from the light chain variable region of the same antibody clone of Table 1.

38-55. (canceled)

56. A method for evaluating a sample from a subject, the method comprising contacting a biological sample from the subject, or extract thereof, with at least one antibody, antigen binding fragment comprising a heavy chain variable region and a light chain variable region, wherein the heavy chain variable region comprises a HCDR1, HCDR2, and HCDR3 having at least 80% sequence identity to the HCDR1, HCR2, HCR3 from a heavy chain variable region of an antibody clone of Table 1 and wherein the light chain variable region comprises a LCDR1, LCDR2, and LCDR3 having at least 80% sequence identity to the LCDR1, LCDR2, and LCDR3 from the light chain variable region of the same antibody clone of Table 1.

57-72. (canceled)
